(19) United States
(12) Patent Application Publication
(10) Pub. No.: US 2023/0218778 A1
(43) Pub. Date: Jul. 13, 2023

(54) PARA-AMINO-BENZYL LINKERS, PROCESS FOR THEIR PREPARATION AND THEIR USE IN CONJUGATES

(71) Applicant: LES LABORATOIRES SERVIER, SURESNES CEDEX (FR)

(72) Inventors: Patrice DESOS, Bois-Colombes (FR); Georges-Alain FRANZETTI, Paris (FR); Jérôme-Benoît STARCK, Rueil-Malmaison (FR); Vesela KOSTOVA, Saint Cloud (FR)

(21) Appl. No.: 17/998,368

(22) PCT Filed: May 18, 2021
(86) PCT No.: PCT/EP2021/063195

§ 371 Date: Nov. 10, 2022

(30) Foreign Application Priority Data
May 19, 2020 (EP) 20315247.5
Nov. 24, 2020 (EP) 20209379.5

(51) Int. Cl. A61K 47/68

(52) U.S. Cl. CPC A61K 47/6889; A61K 47/6855

(57) ABSTRACT

The present invention relates to para-amino-benzyl linker compounds useful for linking drug moieties to antibodies, to linker-drug compounds in which said para-amino-benzyl linker compounds are covalently linked to drug moieties, and to antibody-drug conjugates in which said para-amino-benzyl linker compounds are covalently linked to drug wherein said drug is enzymatically cleaved from the conjugate at a particular cell or tissue type targeted by said antibody.

The present invention relates to para-amino-benzyl linker compounds useful for linking drug moieties to antibodies, to linker-drug compounds in which said para-amino-benzyl linker compounds are covalently linked to drug moieties, and to antibody-drug conjugates comprising said para-amino-benzyl linker compounds covalently linked to drug wherein said drug is enzymatically cleaved from the conjugate at a particular cell or tissue type targeted by said antibody.

### BACKGROUND OF THE INVENTION

Targeted therapeutics are designed to reduce nonspecific toxicities and increase, efficacy relative to conventional therapy. This approach is embodied by the powerful targeting ability of monoclonal antibodies or antigen-binding fragments to specifically deliver highly potent, conjugated small molecule therapeutics to antigen-positive cells. To address the issue of toxicity, chemotherapeutic agents (drugs) have been coupled to targeting molecules such as antibodies or protein receptor ligands that bind with a high degree of specificity to tumor cells to form compounds referred to as antibody-drug conjugates (ADC) or immunoconjugates (Chau et al. Lancet 2019, 394, 793-804). ADC or immunoconjugates in theory should be less toxic because they direct the cytotoxic drug to disease cells that overexpress the particular cell surface antigen or receptor compared to normal cells. Promising advancements with ADC or immunoconjugates have seen cytotoxic drugs linked to antibodies through a linker that is cleaved at the tumor site or inside tumor cells. Such advancements can be applied to any diseases needing a targeting delivery (Tumey, Innovations for Next-Generation Antibody-Drug Conjugates, Humana Press 2018, 187-214) Recent examples show also an interest in immune-mediated inflammatory diseases (Wang et al. J Am Client Soc. 2015, 137, 3229-32), muscle-related diseases (Sugo et al. J. Control Release 2016, 237, 1-13) and diseases caused by bacteria infection (Mariathasan et al. Trends Mol Med. 2017, 23, 135-149). Moreover, ADCs are not restricted to cytotoxic drugs and can be used to deliver specifically imaging agents (Dammes et al. Theranostics 2020, 10, 938-955) or oligonucleotides (Dovgan et al. Bioconjug. Chem. 2019, 30, 2483-2501) to antigen positive cells.

Linkers may impact the physico-chemical properties of an ADC. As many cytotoxic agents are hydrophobic in nature, linking them to the antibody with an additional hydrophobic moiety may lend to aggregation, ADC aggregates are insoluble and often limit achievable drug loading onto the antibody, which can negatively affect the potency of the ADC. Protein aggregates of biologics, in general, have also been linked to increased immunogenicity.

A chemical solution to targeted delivery of drugs, e.g. cytotoxic or cytostatic drugs, conjugated to cell-specific ligands is the “self-immolative linker”, PABC or PAB (para-aminobenzyloxycarbonyl or para-aminobenzyl), attaching the drug moiety to the ligand in the conjugate (Alouane et al. Angew. Chem. Int. Ed. 2015, 54, 7492-7509; Bargh et al. Chem Soc. Rev. 2019, 48, 4361-4374). The PAB linker unit is also referred to as an electronic cascade spacer. The amide bond linking the carboxy terminus of a peptide unit and the para-aminobenzyl of PABC or PAB may be a substrate and cleavable by certain proteases. The aromatic amine becomes electron-donating and initiates art electronic cascade that leads to the expulsion of the leaving group, which releases the free drug after elimination of carbon dioxide (de Groot et al. J. Org. Chem. 2001, 66, 8815-8830). Upon cleavage of a peptide bond adjacent to the PABC or PAB, i.e. by an intracellular enzyme, the drug is released from the ligand whereby no remaining portion of the linker is bound (de Groot et al. Molecular Cancer Therapeutics 2002, 1, 901-911; de Groot et al. J. Med Chem 1999, 42, 5277-5283).

Linkers containing the para-aminobenzyl or para-aminobenzyloxycarbonyl (PAB or PABC) unit, in conjunction with a peptide unit, have been developed with a “self-immolating” or “self-immolative” mechanism of 1,6-elimination and fragmentation under enzymatic, hydrolytic, or other metabolic conditions to release a drug moiety from a targeting ligand, such as an antibody (EP 1 718 667; WO 2016/040684). For the use of the PAB unit in prodrugs and conjugates, see also: Alouane et al. Angew. Chem. Int. Ed. 2015, 54, 7492-7509; Bargh et al. Chem. Soc. Rev. 2019, 48, 4361-4374; Dal Corso et al. Chem. Eur. J. 2019, 25, 14740-14757.

Limitations of the PAB type self-immolating linkers are the propensity to cause poor solubility and aggregation of the conjugates. For example, in WO 2017/214282, PAB type self-immolating linkers containing several hydrophilic groups (such as glucoronides, polyhydroxylated side chains . . . ) lead to high aggregation of conjugates (>15%). In addition, some PAB-containing conjugates may not be suitable substrates for certain cleaving enzymes or cleave too slowly to achieve efficacy. Consequently, it would be desirable to improve the properties of antibody-drug conjugates by optimizing the structure of a self-immolative linker in order to reduce aggregation, while keeping pharmacological activity and stability. Unexpectedly, these novel para-amino-benzyl linkers, comprising a para-amino-benzyl unit and a peptide unit as disclosed herein, address the above-mentioned drawbacks and their use result in ADCs with low aggregation levels, good stability, desirable levels of drug loading and expected pharmacological activity, as demonstrated in the following Examples.

### BRIEF DESCRIPTION OF THE FIGURES

FIG. 1 shows site-specific antibody conjugation using bacterial transglutaminase (LAR2).

FIG. 2 shows conjugation using 4 inter-chain disulfide bridges of the antibody.

FIG. 3 shows conjugation using rebridging technology generating fragmentation.

### DEFINITIONS

The term “antibody” or “Ab” is used in the broadest sense to refer to an immunoglobulin molecule that recognizes and specifically binds to a target, such as a protein, polypeptide, carbohydrate, polynucleotide, lipid, or combinations of the foregoing through at least one antigen recognition site within the variable region of the immunoglobulin molecule. An antibody can be polyclonal or monoclonal, multiple or single chain, or an intact immunoglobulin, and may be derived from natural sources or from recombinant sources. An “intact” antibody is a glycoprotein that typically comprises at least two heavy (H) chains and two light (L) chains inter-connected by disulfide bonds. Each heavy chain is comprised of a heavy chain variable region (abbreviated herein as VH) and a heavy chain constant region. The heavy chain constant region comprises three domains, CH1, CH2 and CR3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g. IgG1, IgG2, IgG3, IgG4, IgA1 and IgA2), or subclass. An antibody can be an intact antibody or an antigen-binding fragment thereof.

The term “antibody fragment” or “antigen-binding fragment” or “fragment” as used herein, refers to at least one portion of an antibody that retains the ability to specifically interact with (e.g., by binding, steric hinderance, stabilizing/destabilizing, spatial distribution) an epitope of an antigen. Antigen-binding fragments may also retain the ability to internalize into an antigen-expressing cell. In some embodiments, antigen-binding fragments also retain immune effector activity. The terms antibody, antibody fragment, antigen-binding fragment, and the like, are intended to embrace the use of binding domains from antibodies in the context of larger macromolecules such as ADCs. It has been shown that fragments of a full-length antibody can perform the antigen binding function of a full-length antibody. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen-binding fragment can also be incorporated into single domain antibodies, maxibodies, minibodies, nanobodies, intrabodies, diabodies, triabodies, tetrabodies, bispecific or multi-specific antibody constructs, ADCs, v-NAR and bis-scFv (see, e.g., Holliger and Hudson Nat. Biotechnol. 2005, 23, 1126-1136). Antigen-binding fragments can also be grafted into scaffolds based on polypeptides such as a fibronectin type III (Fn3) (see U.S. Pat. No. 6,703,199, which describes fibronectin polypeptide minibodies).

The term “scFV” refers to a fusion protein comprising at least one antigen-binding fragment comprising a variable region of a light chain and at least one antigen-binding fragment comprising a variable region of a heavy chain, wherein the light and heavy chain variable regions are contiguously linked e.g., via a synthetic linker, e.g., a short flexible polypeptide linker, and capable of being expressed as a single chain polypeptide, and wherein the scFv retains the specificity of the intact antibody from which it is derived. Unless specified, an scFv may have the VL and VH variable regions in either order, e.g., with respect to the N-terminal and C-terminal ends of the polypeptide, the scFv may comprise VL-linker-VH or may comprise VH-linker-VL. Antigen-binding fragments are obtained using conventional techniques known to those of skill in the art, and the binding fragments are screened for utility (e.g., binding affinity, internalization) in the same manner as are intact antibodies. Antigen-binding fragments, for example, may be prepared by cleavage of the intact protein, e.g., by protease or chemical cleavage.

The term “drug” or “agent” or “therapeutic drug” or “therapeutic agent”, as used herein, refers to a chemical compound, a mixture of chemical compounds, a biological macromolecule, an extract made from biological materials, or a combination of two or more thereof, that is capable of modulating a biological process and or has biological activity. Particularly, the term “drug” can represent a “chemotherapeutic agent” or an “anti-cancer agent”.

The term “chemotherapeutic agent” or “anti-cancer agent” is used herein to refer to all agents that are effective in treating cancer (regardless of mechanism of action). Inhibition of metastasis or angiogenesis is frequently a property of a chemotherapeutic agent. Chemotherapeutic agents include antibodies, biological molecules, and small molecules. A chemotherapeutic agent may be a cytotoxic or cytostatic agent. Examples of chemotherapeutic agent include, without implying any limitation, monomethyl auristatin E, auristatin E, SN-38, doxorubicin, etc.

The term “cytostatic agent” refers to an agent that inhibits or suppresses cell growth and/or multiplication of cells.

The term “cytotoxic agent” refers to a substance that causes cell death primarily by interfering with a cell's expression activity and/or functioning.

As used herein the term “halo(C₁-C₄)alkyl” means (C₁-C₄)alkyl radical which is substituted by one or more halogen atoms such as fluorine, chlorine, bromine or iodine including, but not limited to, fluoromethyl, difluoromethyl, trifluoromethyl, 2-fluoroethyl, 2,2-difluoroethyl, 2,2,2-trifluoroethyl, 2,2,2-trifluoro-1,1-dimethylethyl, 2,2,2-trichloroethyl, 3-fluoropropyl, 4-fluorobutyl, chloromethyl, trichloromethyl, iodomethyl and bromomethyl.

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (Boc), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable hydroxy-protecting groups include acetyl benzyl, benzoyl, tetrahydropyranyl, and trialkylsilyl. Also, a “carbonyl-protecting group” refers to a substituent of a carbonyl group that blocks or protects the carbonyl functionality. Suitable carbonyl-protecting groups include acetal, acylal, and dithianes. Similarly, a “carboxyl-protecting group” refers to a substituent of a carboxyl group that blocks or protects the carboxyl functionality. Suitable carboxyl-protecting groups include methyl ester, benzyl ester, tert-butyl ester, silyl ester, and orthoester. For a general description of protecting groups and their use, see T. W. Greene, Protective Groups in Organic Synthesis, John Wiley & Sons, New York, 1991.

The term “moiety” as used herein means a specified segment, fragment or functional group of a molecule or compound. Chemical moieties are sometimes indicated as chemical entities that are embedded in or appended (i.e., a substituent or variable group) to a molecule, compound or chemical formula.

The term “bridging spacer”, as used herein, refers to one or more linker components which are covalently attached together to form a bivalent moiety which links the bivalent peptide A₁-A₂ to the reactive group. In certain embodiments, the “bridging spacer” comprises a carbonyl group attached to the N-terminus of the A₂ group via an amide bond. In certain embodiments, the “bridging spacer” comprises an amino group attached to the carbonyl group of the A₂ group via an amide bond. In some embodiments, the bridging spacer comprises a polyoxyethylene (PEG) group. In other embodiments, the bridging spacer comprises a butanoyl, pentanoyl, hexanoyl, heptanoyl, or octanoyl group. In some embodiments, the bridging spacer comprises a hexanoyl group (i.e. a —CO—(CH₂)₅— group). In preferred embodiments, the bridging spacer may comprise: —CO—CH₂—CH₂—PEG1—, —CO—CH₂—PEG3—, or —NH—CH₂—CH₂—PEG1—.

The term “polyoxyethylene”, “polyethylene glycol” or “PEG” as used herein, refers to a linear chain, a branched chain or a star shaped configuration comprised of (OCH₂CH₂) groups. In certain embodiments, a polyoxyethylene or PEG group is *—(OCH₂CH₂)ₜ—**, where t is 1-20, and where the “*” indicates the end directed toward the A₂-A₁ group and the “**” indicates the point of attachment to a reactive group. In some embodiments, the bridging spacer comprises a PEG group which is *—(OCH₂CH₂ₜ—**, where t is 1-20, and where the “*” indicates the point of attachment to a —(CH₂)ₙ—C(O)— group wherein the carbonyl group is attached to the N-terminus of the A₂ group and n is 1-5, and the “**” indicates the point of attachment to a reactive group. In some embodiments, the bridging spacer comprises a PEG group which is *—(OCH₂CH₂)ₜ—**, where t is 1-20, and where the “*” indicates the point of attachment to a —(CH₂)ₙ—NH— group wherein the amino group is attached to the carbonyl group of the A₂ group and n is 1-5, and the “**” indicates the point of attachment to a reactive group. For example, the term “PEG3” as used herein means that t is 3.

The term “reactive group”, as used herein, is a functional group capable of forming a covalent bond with a functional group of an antibody or an antibody fragment. In some embodiments, the reactive group is selected from, but are not limited to, a thiol, a maleimide, a dibromomaleimide, a haloacetamide, an azide, an alkyne, a cyclcooctene, a triaryl phosphine, an oxanobornadiene, a cyclooctyne, a diaryl tetrazine, a monoaryl tetrazine, a norbornene, an aldehyde, a hydroxylamine, a hydrazine, NH₂—NH—C(═)—, a ketone, a vinyl sulfone, an aziridine, an amino, an amino acid residue. . . In a preferred embodiment, the reactive group is selected from a maleimide group, a dibromomaleimide group, a thiol group, a cyclooctyne group, or an azido group, more preferably, a maleimide group, a dibromomaleimide group or an azido group. For example, maleimide or dibromomaleimide groups may have the structure:

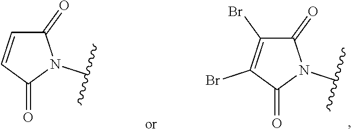 [SMILES: *N1C(=O)C=CC1=O ; *N1C(=O)C(Br)=C(Br)C1=O]

respectively.

For example, the azido group may have the structure: —N═N⁺═N⁻.

The term “attachment group ”, as used herein, refers to a bivalent or trivalent moiety which links the bridging spacer to the antibody or fragment thereof. The attachment group is a bivalent or trivalent moiety formed by the reaction between a reactive group and a functional group on the antibody or fragment thereof. In some embodiments, the reactive group for the formation of the attachment group is selected from, but are not limited to, a thiol, a maleimide, a dibromomaleimide, a haloacetamide, an azide, an alkyne, a cyclooctene, a triaryl phosphine, an oxanobornadiene, a cyclooctyne, a diaryl tetrazine, a monoaryl tetrazine, a norbornene, an aldehyde, a hydroxylamine, a hydrazine, NH₂—NH—C(═O)—, a ketone, a vinyl sulfone, an aziridine, an amino, an amino acid residue . . . In a preferred embodiment, the reactive group for the formation of the attachment group is selected from a maleimide group, a dibromomaleimide group, a thiol group, a cyclooctyne group, or an azido group, more preferably, a maleimide group, a dibromomaleimide group or an azido group.

The term “Spacer Unit precursor” or “Spacer Unit precursor Z′” or “Z′”, as used herein, refers to a component comprising a bridging spacer and a reactive group.

The term “Spacer Unit” or “Spacer Unit Z” or “Z”, as used herein, refers to a component comprising a bridging spacer and an attachment group.

The term “conjugate” or “antibody-drug conjugate” or “ADC” or “immunoconjugate”, as used herein, refers to an antibody which is covalently attached to a drug moiety through a para-amino-benzyl linker of Formula (I). In one embodiment, antibody-drug conjugate of Formula (III) wherein the Spacer Unit Z is coupled to antibody Ab via an amide group may be prepared by reacting a free amine functional group on antibody Ab with an active ester containing precursor of Spacer Unit Z. For example, a carboxyl group on Spacer Unit precursor Z′ may be activated by reacting with N-hydroxysuccinimide and then reacted with Ab—NH₂ to form a conjugate in which Ab and Z are coupled by way of an amide group. Useful functional groups on an antibody for linking to the Spacer Unit Z, either naturally or via chemical manipulation include, but are not limited to, sulfhydryl (—SH), amino, hydroxyl, the anomeric hydroxyl group of a carbohydrate, and carboxyl. In one embodiment, the reactive functional groups on the antibody are sulfhydryl and amino. Sulfhydryl groups can be generated by reduction of an intramolecular cysteine disulfide bond of an antibody. Alternatively, sulfhydryl groups can be generated by reaction of an amino group of a lysine moiety of an antibody using 2-iminothiolane (Traut's reagent) or another sulfhydryl generating reagent. In another embodiment, the Spacer Unit Z is linked to the antibody Ab via a disulfide bond between a sulfur atom of the antibody and a sulfur atom of the Spacer Unit Z. In yet another embodiment, the reactive group of the Spacer Unit Z contains a reactive site that can form a bond with a primary or secondary amino group of an antibody. Example of these reactive sites include, but are not limited to activated esters such as succinimide esters, 4-nitrophenyl esters, pentafluorophenyl esters, tetrafluorophenyl esters, anhydrides, acid chlorides, sulfonyl chlorides, isocyanates and isothiocyanates. In yet another embodiment, the reactive group of the Spacer Unit Z reacts with an aldehyde, acetal, or ketal group on a sugar (carbohydrate) of a glycosylated antibody. For example, a carbohydrate can be mildly oxidized using a reagent such as sodium periodate and the resulting (—CHO) unit of the oxidized carbohydrate can be condensed with a Spacer Unit that contains a functionality such as a hydrazide, an oxime, a primary or secondary amine, a hydrazine, a thiosemicarbazone, a hydrazine carboxylate, and an arylhydrazide such as those described by Kaneko et al. Bioconjugate Chem. 1991, 2, 133-141.

The generation of the ADCs can be accomplished by techniques known to the skilled artisan.

In an exemplary embodiment, the invention provides for an ADC of Formula (III) prepared from contacting a Linker-Drug compound of Formula (II) with an antibody (or fragment thereof) having a reactive sulfhydryl, amino or carboxyl moiety under suitable conditions to effect condensation of the reactive moiety with the Spacer Unit precursor Z′ moiety of the Linker-Drug compound of Formula (II), wherein Z′ is converted to Z resulting from said contact.

In some embodiment, the ADC of Formula (III) incorporates a Spacer Unit Z which is a divalent moiety that couples the N-terminus of the A₂-A₁ group to the antibody. In some embodiment, the ADC of Formula (III) incorporates a Spacer Unit Z which is a divalent moiety that couples the carbonyl group of the A₂-A₁ group to the antibody.

The Spacer Unit Z is covalently bound to a functional group pending from the antibody such as an amine (e.g. —NH₂ from a Lys residue), a carboxyl (—COOH from an Asp or Glu residue) or a sulfhydryl (e.g. —SH from a Cys residue) which forms an amide or a thioether or disulfide group. Antibody-drug conjugate of the invention in which the Spacer Unit precursor Z′ is reacted with a sulfhydryl functional group of the antibody (for example, Cys containing peptide or a reduced antibody) to form a thioether linkage include the one represented by the following formula:

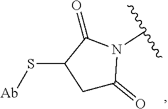

wherein Ab is an antibody or an antigen-binding fragment thereof. In other embodiment, antibody-drug conjugate of the invention in which the Spacer Unit precursor Z′ is reacted with two sulfhydryl functional groups of the antibody to form two thioether linkages include the one represented by the following formula:

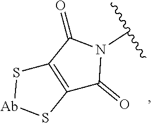

wherein Ab is an antibody or an antigen-binding fragment thereof.

In some embodiments, the antibody or antigen-binding fragment is functionalized to prepare a functional group that is reactive with a Linker-Drug intermediate. In some embodiments, an antibody or antigen-binding fragment is prepared with bacterial transglutaminase (BTG) which are reactive glutamines specifically functionalized with an amine containing cyclooctyne BCN (N-[(1R,8S9s)-Bicyclo[6.1.0]non-4-yn-9-ylmethyloxycarbonyl]-1,8-diamino-3,6-dioxaoctane) moiety. In some embodiments, ADC of the invention in which the Spacer Unit precursor Z′ is reacted with a glutamine functional group of antibody to form an amide linkage include the one represented by the following formula:

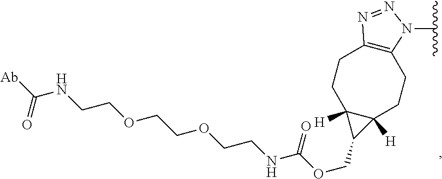

wherein Ab is an antibody or an antigen-binding fragment thereof.

The term “drug loading” is represented by p, and is also referred to herein as the drug-to-antibody ratio (DAR or drug:antibody ratio). Drug loading may range from 1 to 8 drug moieties per antibody or antigen-binding fragment. In some embodiments, higher drug loading (e.g., p>8) may cause aggregation, insolubility, toxicity, or loss of cellular permeability of certain antibody-drug conjugates. Higher drug loading may also negatively affect the pharmacokinetics (e.g., clearance) of certain ADCs. In some embodiments, lower drug loading (e.g., p<2) may reduce the potency of certain ADC's against target-expressing cells. In some embodiments, p is an integer from 2 to 8. In some embodiments, is 2, or p is 4, or p is H.

The drug loading of an ADC may be controlled in different ways, e.g., but not limited to, by limiting the molar excess of drug-linker intermediate or linker reagent relative to antibody, or by limiting the conjugation reaction time or temperature.

In some embodiments, the drug loading in a mixture of ADCs resulting from a conjugation reaction ranges from 1 to 8 drug moieties attached per antibody or antigen-binding fragment. The average number of drug moieties per antibody or antigen-binding fragment (i.e., the average drug loading, or average p) may be calculated by any conventional method known in the art e.g., by mass spectrometry (e.g., liquid chromatography-mass spectrometry (LC-MS)) and/or high-performance liquid chromatography (e.g., HIC-HPLC). In some embodiments, the average number of drug moieties per antibody or antigen-binding fragment is from about 1.5 to about 2.5, or about 7.0 to about 8. In some embodiments, the average number of drug moieties per antibody or antigen-binding fragment is about 2. In some embodiments, the average number of drug moieties per antibody or antigen-binding fragment is 2. In some embodiments, the average number of drug moieties per antibody or antigen-binding fragment is about 4. In some embodiments, the average number of drug moieties per antibody or antigen-binding fragment is 4. In some embodiments, the average number of drug moieties per antibody or antigen-binding fragment is about 8. In some embodiments, the average number of drug moieties per antibody or antigen-binding fragment is 8.

### DETAILED DESCRIPTION OF THE INVENTION

According to a first aspect of the invention, there is provided a para-amino-benzyl linker compound of Formula (I):

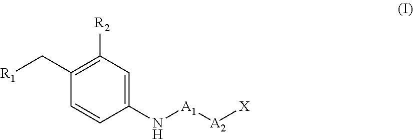

wherein:
R₁ represents a hydroxy group or a halogen atom;
R₂ represents a —S(O)₂(OH) group, a —S(O)₂(O⁻M⁺) group, a linear or branched —(C₁-C₄)alkyl-S(O)₂(OH) group, a linear or branched —(C₁-C₄)alkyl-S(O)₂(O⁻M⁺) group, a linear or branched -halo(C₁-C₄)alkyl-S(O)₂(OH) group, or a linear or branched -halo(C₁-C₄)alkyl-S(O)₂(O⁻M⁺) group;
A₁ represents a —C(O)—CH(R₃)—NH— group;
A₂ represents a —C(O)—CH(R₄)—NH— group, a

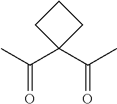

group, a

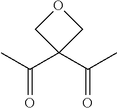

group, or a

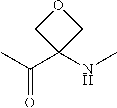

group;
R₃ and R₄, independently of one another, represent the side chain of an amino acid;
X represents a hydrogen atom, a hydroxy group, or a protecting group;
M⁺ represents a pharmaceutically acceptable monovalent canon.

In a preferred embodiment, R₁ represents a hydroxy group, a bromine atom, a chlorine atom, or an iodine atom. More preferably, R₁ represents a hydroxy group, a chlorine atom, or an iodine atom. Even more preferably, R₁ represents a hydroxy group.

Preferably, R₂ represents a —S(O)₂(OH) group, a —S(O)₂(O⁻M⁺) group, a —CH₂—S(O)₂(OH) group, a —CH₂—S(O)₂(O⁻M⁺) group, a —CH₂—CH₂—S(O)₂(OH) group, a —CH₂—CH₂—S(O)₂(O⁻M⁺) group, a —CH₂—CH₂—CH₂—S(O)₂(OH) group, or a —CH₂—CH₂—CH₂—S(O)₂(O⁻M⁺) group. Examples of M⁺ include Li⁺, Na⁺, or K⁺. In a preferred embodiment, M⁺ represents Na⁺.

In a particular embodiment, A₁ represents a —C(O)—CH(R₃)—NH— group and A₂ represents a —C(O)—CH(R₄)—NH— group, in which R₃ and R₄, independently of one another, represent the side chain of an amino acid. The side chain-bearing carbon may be in either D or L (R or S) configuration. Preferably, the side chain-bearing carbon is L configuration. In a particular embodiment, R₃ and R₄, independently of one another, represent, but are not limited to, hydrogen, methyl, n-propyl, isopropyl, n-butyl, isobutyl, sec-butyl, phenyl, benzyl, p-hydroxybenzyl, —CH₂OH, —CH₂SH, —CH₂SeH —CH₂CH₂OH, —CH₂CH₂SH, —CH(OH)CH₃, —CH₂SCH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCO—(3-methyl-3,4-dihydro-2H-pyrrol-2-yl), —(CH₂)₄NHCO—(3,4-dihydro-2H-pyrrol-2-yl), —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 4-imidazolylmethyl, 3-indolylmethyl, . . .

In a preferred embodiment, A₁-A₂ group form a dipeptide containing 2 amino acid units, wherein said amino acid units are selected from natural and non-natural amino acids, preferably natural amino acids. Said amino acid unit may be alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucine (Nle), selenocysteine (Sec), pyrrolysine (Pyl), homoserine, homocysteine, and desmethyl pyrrolysine. In one embodiment, when A₁ represents a proline (Pro) amino acid unit, A₁ is defined by the formula:

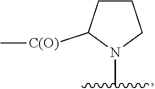

wherein the carbonyl group is attached to the amino group of the para-amino-benzyl moiety and the wavy line indicates the covalent attachment site to the carbonyl group of the A₂ group.

In one embodiment, when A₂ represents a proline (Pro) acid unit, A₂ is defined by the formula:

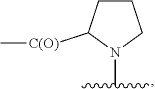

wherein the carbonyl group is attached to the amino group of the A₁ group and the wavy line indicates the covalent attachment site to the X group, Z′ group or Z group as defined in the invention.

In a particular embodiment, A₁-A₂ group can represent Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-ala; Ala-Phe; Phe-Lys; Lys-Phe; val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; Trp-Cit. In a preferred embodiment, A₁-A₂ group represents Cit-Val or Ala-Val.

Preferably, A₁ represents a —C(O)—CH(R₃)—NH— group in which R₃ represents a —(CH₂)₃—NH—CO—NH₂ group or a methyl group.

Advantageously, A₂ represents a —C(O)—CH(R₄)—NH— group in which R₄ represents an isopropyl group; a

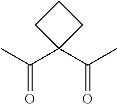

group; a

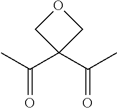

group; or a

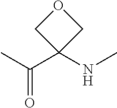

group.

In a preferred embodiment, A₁ represents a —C(O)—CH(R₃)—NH— group in which R₃ represents a —(CH₂)₃—NH—CO—NH₂ group or a methyl group and A₂ represents a —C(O)—CH(R₄)—NH— group in which R₄ represents an isopropyl group.

In another preferred embodiment, Aₜ represents a —C(O)—CH(R₃)—NH— group in which R₃ represents a —(CH₂)₃—NH—CO—NH₂ group, and A₂ represents a

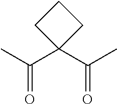

group or a

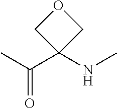

group.

In one embodiment, X represents a hydrogen atom or a protecting group. Preferably, X represents a hydrogen atom, or a protecting group for amino functionality, carbonyl functionality, or carboxyl functionality. More preferably, X represents a hydrogen atom, or a protecting group selected from Fmoc, Boc, or CBz. Even more preferably, X represents a Fmoc protecting group.

Preferred para-amino-benzyl linker compound of Formula (I) are:
sodium 5-[[(2S)-2-[[(2S)-2-(9H-fluoren-9-ylmethoxycarbonylamino)-3-methyl-butanoyl]amino]-5-ureido-pentanoyl]amino]-2-(hydroxymethyl)benzenesulfonate;
sodium 5-[[(2S)-2-[[(2S)-2-(9H-fluoren-9-ylmethoxycarbonylamino)-3-methyl-butanoyl]amino]propanoyl]amino]-2-(hydroxymethyl)benzenesulfonate;
5-[[(2S)-2-[[(2S)-2-(9H-fluoren-9-ylmethoxycarbonylamino)-3-methyl-butanoyl]amino]propanoyl]amino]-2-(hydroxymethyl)benzenesulfonic acid;
sodium [5-[[(2S)-2-[[(2S)-2-(9H-fluoren-9-ylmethoxycarbonylamino)-3-methyl-butanoyl]amino]-5-ureido-pentanoyl]amino]-2-(hydroxymethyl)phenyl]methanesulfonate;
2-chloromethyl)-5-[[(2S)-2-[[(2S)-2-(9H-fluoren-9-ylmethoxycarbonylamino)-3-methyl-methyl-butanoyl]amino]-5-ureido-pentanoyl]amino]benzenesulfonic acid;
2-(chloromethyl)-5-[[(2S)-2-[[(2S)-2-(9H-fluoren-9-ylmethoxycarbonylamino)-3-methyl-methyl-butanoyl]amino]propanoyl]amino]benzenesulfonic acid;
5-[[(2S)-2-[[(2S)-2-(9H-fluoren-9-ylmethoxycarbonylamino)-3-methyl-butanoyl]amino]-5-ureido-pentanoyl]amino]-2-(iodomethyl)benzenesulfonic acid;
sodium N-{[(9H-fluoren-9-yl)methoxy]carbonyl}-L-valyl-N⁵-carbamoyl-N-[4-(hydroxymethyl-3-(2-sulfonatoethyl)phenyl]-L-ornithinamide;
N-{[(9H-fluoren-9-yl)methoxy]carbonyl}-L-valyl-N⁵-carbamoyl-N-[4-(hydroxymethyl-3-(3-sulfonatoethyl)phenyl]-L-ornithinamide;

The invention relates also to a process for the preparation of para-amino-benzyl linker compounds of formula (II), which process is characterized in that there is used as starting material the compound of Formula (IV):

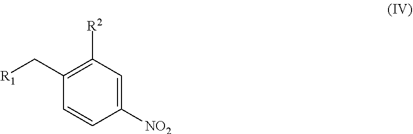

wherein R₁ and R₂ are as defined for Formula (I).
which is subjected to a reduction reaction to yield the compound of Formula (V):

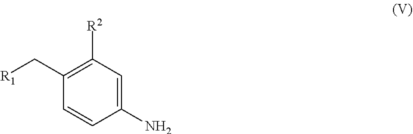

wherein R₁ and R₂ are as defined herein before,
compound of Formula (V) which is further subjected to coupling with the compound of Formula (VI):

HO—A₁—P (VI)

wherein P is an amino-protecting group and A₁ is as defined in Formula (I),
to yield the compound of Formula (VII)

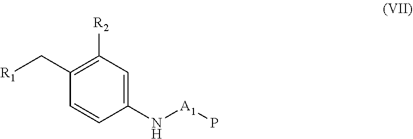

wherein A₁, P, R₁ and R₂ are as defined herein before.
compound of Formula (VII) which is further subjected to coupling with the compound of Formula (VIII):

HO—A₂—X (VIII)

wherein X and A₂ are as defined in Formula (I),
to yield the para-amino-benzyl linker compound of Formula (I).

An alternative process for the preparation of para-amino-benzyl linker compounds of Formula (I) is characterized in that there is used as starting material the compound of Formula (V), obtained from compound of Formula (IV), which is subjected to coupling with the compound of Formula (IX):

HO—A₁—A₂—X (IX)

wherein A₁, A₂ and X are as defined in Formula (I),
to yield the para-amino-benzyl linker compound of Formula (I).

In one embodiment para-amino-benzyl linker compound of Formula (I) obtained at the end of the above-mentioned processes may be further purified according to a conventional separation technique, which are converted, if desired, into their addition salts with a pharmaceutically acceptable acid or base and which may be separated into their isomers according to a conventional separation technique, it being understood that at any moment considered appropriate during the course of the process described above, some groups (hydroxy, amino . . . ) of the starting reagents or of the synthesis intermediates can be protected subsequently deprotected and functionalized as required by the synthesis. The compounds of Formulae (IV), (VI), (VIII) and (IX) are either commercially available or can be obtained by the person skilled in the art using conventional chemical reactions described in the literature.

The invention relates also to the use of para-amino-benzyl linker compounds of Formula (I), in the preparation of an antibody-drug conjugate. More particularly, the invention relates to the use of para-amino-benzyl linker compound of Formula (I) selected from:
sodium 5-[[(2S)-2-[[(2S)-2-(9H-fluoren-9-ylmethoxycarbonylamino)-3-methyl-butanoyl]amino]-5-ureido-pentanoyl]amino]-2-(hydroxymethyl)benzenesulfonate,
sodium 5-[[(2S)-2-[[(2S)-2-(9H-fluoren-9-ylmethoxycarbonylamino)-3-methyl-butanoyl]amino]propanoyl]amino]-2-(hydroxymethyl)benzenesulfonate,
5-[[(2S)-2-[[(2S)-2-(9H-fluoren-9-ylmethoxycarbonylamino)-3-methyl-butanoyl]amino]propanoyl]amino]-2-(hydroxymethyl)benzenesulfonic acid,
sodium [5-[[(2S)-2-[[(2S)-2-(9H-fluoren-9-ylmethoxycarbonylamino)-3-methyl-butanoyl]amino]-5-ureido-pentanoyl]amino]-2-(hydroxymethyl)phenyl]methanesulfonate,
2-(chloromethyl)-5-[[(2S)-2-[[(2S)-2-(9H-fluoren-9-ylmethoxycarbonylamino)-3-methyl-butanoyl]amino]-5-ureido-pentanoyl]amino]benzenesulfonic acid, or
2-(chloromethyl)-5-[[(2S)-2-[[(2S)-2-(9H-fluoren-9-ylmethoxycarbonylamino)-3-methyl-butanoyl]amino]propanoyl]amino]benzenesulfonic acid,
5-[[(2S)-2-[[(2S)-2-(9H-fluoren-9-ylmethoxycarbonylamino)-3-methyl-butanoyl]amino]-5-ureido-pentanoyl]amino]-2-(iodomethyl)benzenesulfonic acid,
sodium N-{[(9H-fluoren-9-yl)methoxy]carbonyl}-L-valyl-N⁵-carbamoyl-N-[4-(hydroxymethyl)-3-(2-sulfonatoethyl)phenyl]-L-ornithinamide, or
N-{[(9H-fluoren-9-yl)methoxy]carbonyl}-L-valyl-N⁵-carbamoyl-N-[4-(hydroxymethyl)-3-(3-sulfopropyl)phenyl]-L-ornithinamide,
   in the preparation of an antibody-drug conjugate.

In a second aspect, the invention provides a Linker-Drug compound of Formula (II):

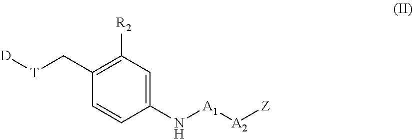

wherein:
D represents a drug moiety;
T is a bond, —O—C(O)—N(CH₃)—CH₂—CH₂—N(CH₃)—C(O)—*, —O—*, —NR₅—*, —NR₅—C(O)—*, or —O—C(O)—*, wherein the * indicates the point of attachment to D;
R₂ represents a —S(O)₂(OH) group, a —S(O)₂(O⁻M⁺) group, a linear or branched —(C₁-C₄)alkyl-S(O)₂OH) group, a linear or branched —(C₁-C₄)alkyl-S(O)₂(O⁻M⁺) group, a linear or branched -halo(C₁-C₄)alkyl-S(O)₂(OH) group, or a linear or branched -halo(C₁-C₄)alkyl-S(O)₂(O⁻M⁺) group;
A₁ represents a —C(O)—CH(R₃)—NH— group;
A₂ represents a —C(O)—CH(R₄)—NH— group, a

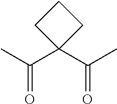

group, a

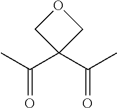

group, or a

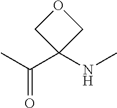

group;
R₃ and R₄, independently of one another, represent the side chain of an amino acid;
R₅ represent a hydrogen atom or a (C₁-C₄)alkyl group;
Z′ represents a Spacer Unit precursor;
M⁺ represents a pharmaceutically acceptable monovalent cation.

In one embodiment in the second aspect of the invention, D is a drug moiety comprising a nitrogen atom or an oxygen atom, wherein D is directly connected to T via the said nitrogen atom or the oxygen atom of the drug moiety. In some embodiment, D is a quaternized tertiary amine-containing drug moiety. In a particular embodiment, D is monomethyl auristatin E (IUPAC Name: (S)-N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide or MMAE), auristatin E (IUPAC Name: (2S)-2-[[(2S)-2-(dimethylamino)-3-methyl-butanoyl]amino]-N-[(1S,2R)-4-[(2S)-2-[(1R,2R)-3-[[(1R,2S)-2-hydroxy-1-methyl-2-phenyl-ethyl]amin]-1-methoxy-2-methyl-3-oxo-propyl]pyrrolidin-1-yl]-2-methoxy-1-[(1S)-1-methylpropyl]-4-oxo-butyl]-N,3-dimethyl-butanamide), SN-38 (IUPAC Name: (4S)-4,11,diethyl-4,9-dihydroxy-1H-pyrano[3′,4′:6,7]indolizino[1,2-b]quinoline-3,14(4H,12H)-dione), or doxorubicin (IUPAC Name: (1S,3S)-3,5,12-trihydroxy-3-(hydroxyacetyl)-10-methoxy-6,11-dioxo-1,2,3,4,6,11-hexahydrotetracen-1-yl 3-amino-2,3,-trideoxy-α-L-lyxo-hexopyranoside).

Preferably, T is a bond or —O—C(O)—*, wherein the * indicates the point of attachment to D. In some embodiments, when T is a bond, D is a quaternized tertiary amine-containing drug moiety.

Preferably in the second aspect of the invention, R₂ represents a —S(O)₂(OH) group, a —S(O)₂(O⁻M⁺) group, a —CH₂—S(O)₂(OH) group, a —CH₂—S(O)₂O⁻M⁺) group, a —CH₂—CH₂—S(O)₂(OH) group, a —CH₂—CH₂—S(O)₂(O⁻M⁺) group, a —CH₂—CH₂—CH₂—S(O)₂(OH) group, or a —CH₂—CH₂—CH₂—S(O)₂(O⁻M⁺) group. In a preferred embodiment of the second aspect of the invention, M⁺ represents Na⁺.

Advantageously in the second aspect of the invention, A₁ represents a —C(O)—CH(R₃)—NH— group in which R₃ represents a —(CH₂)₃—NH—CO—NH₂ group or a methyl group.

In a preferred embodiment of the second aspect of the invention, A₂ represents a —C(O)—CH(R₄)—NH— group in which R₄ represents an isopropyl group; a

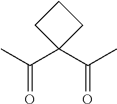

group; a

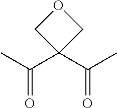

group; or a

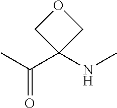

group.

In another preferred embodiment of the second aspect of the invention, A₁ represents a —C(O)—CH(R₃)—NH— group in which R₃ represents a —(CH₂)₃—NH—CO—NH₂ group, and A₂ represents a

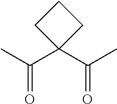

group, or a

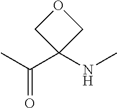

group.

In another preferred embodiment of the second aspect of the invention. Aₜ represents a —C(O)—CH(R₃)—NH— group and A₂ represents a —C(O)—CH(R₄)—NH— group, in which R₃ and R₄, independently of one another, represent the side chain of an amino acid. Particularly, when R₃ and R₄ represent a side chain of an amino acid, the side chain-bearing carbon is L configuration. In a preferred embodiment, A₁ represents a —C(O)—CH(R₃)—NH— group in which R₃ represents a —(CH₂)₃—NH—CO—NH₂ group or a methyl group and A₂ represents a —C(O)—CH(R₄)—NH— group in which R₄ represents an isopropyl group. Preferably in the second aspect of the invention, A₁-A₂ group represents Cit-Val or Ala-Val.

Preferably, Spacer Unit precursor Z′ is selected from:

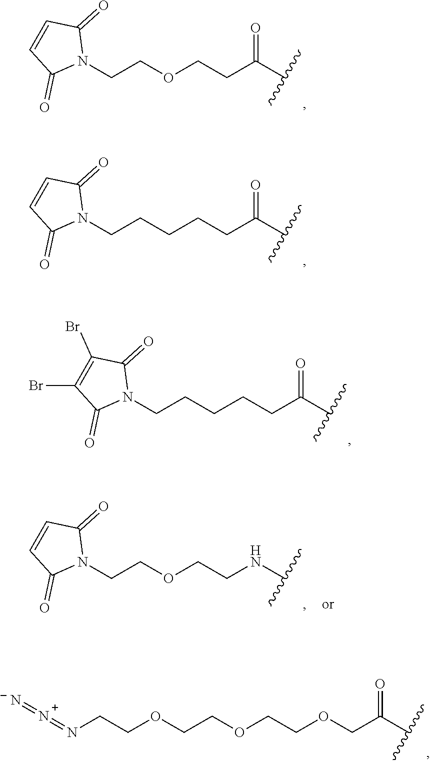 [SMILES: *C(=O)CCOCCN1C(=O)C=CC1=O ; *C(=O)CCCCCN1C(=O)C=CC1=O ; *C(=O)CCCCCN1C(=O)C(Br)=C(Br)C1=O ; *NCCOCCN1C(=O)C=CC1=O ; *C(=O)COCCOCCOCCN=[N+]=[N-]]

wherein the wavy line indicates the covalent attachment site to the N-terminus or the carbonyl group of A₂ group.

Preferred Linker-Drug compound of Formula (II) are selected from:

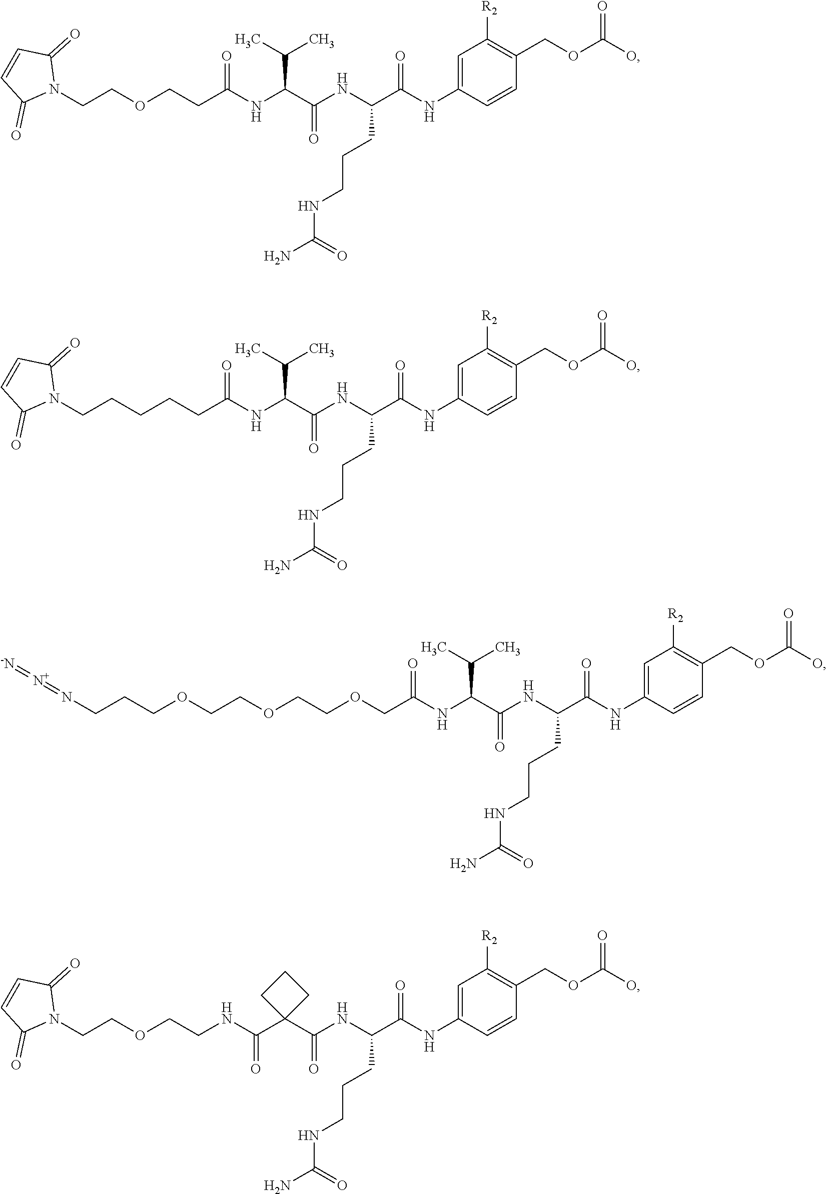

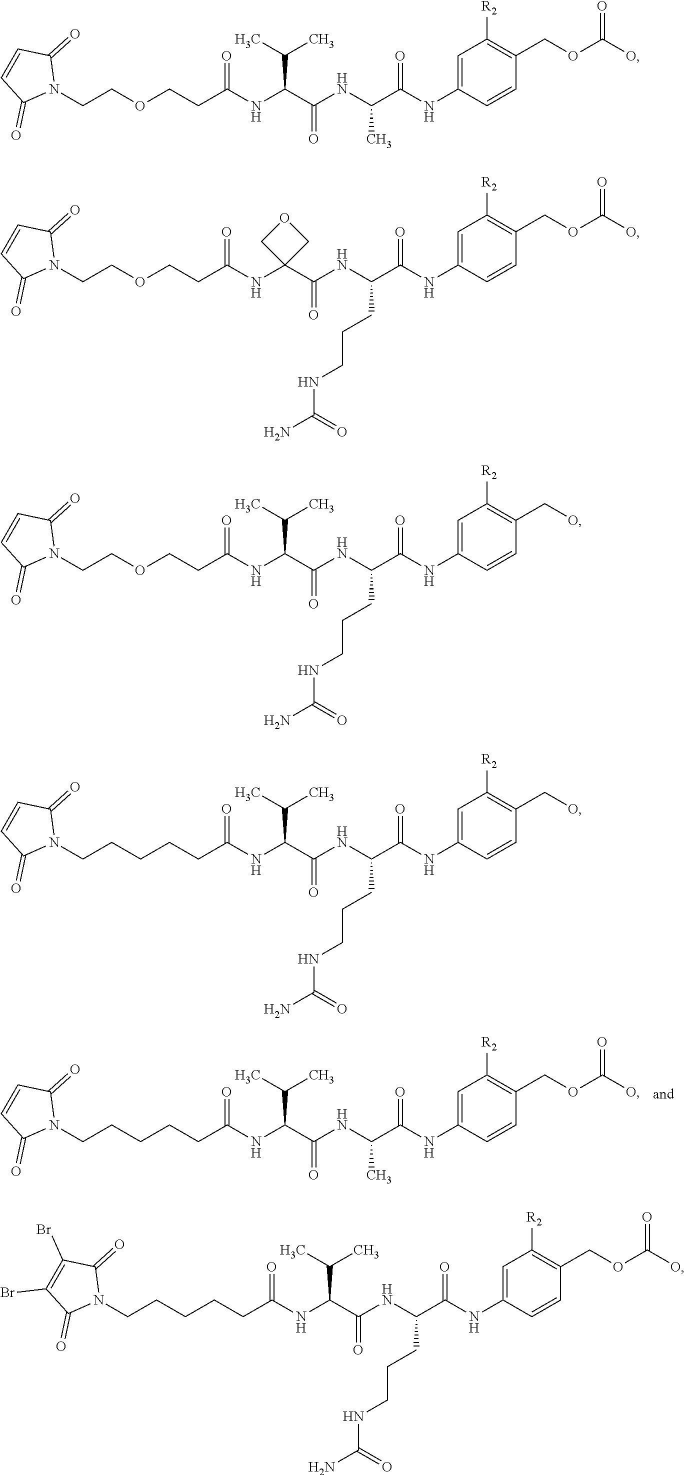

wherein R₂ and D see as defined herein before.

More preferred Linker-Drug compound of Formula (II) are selected from:

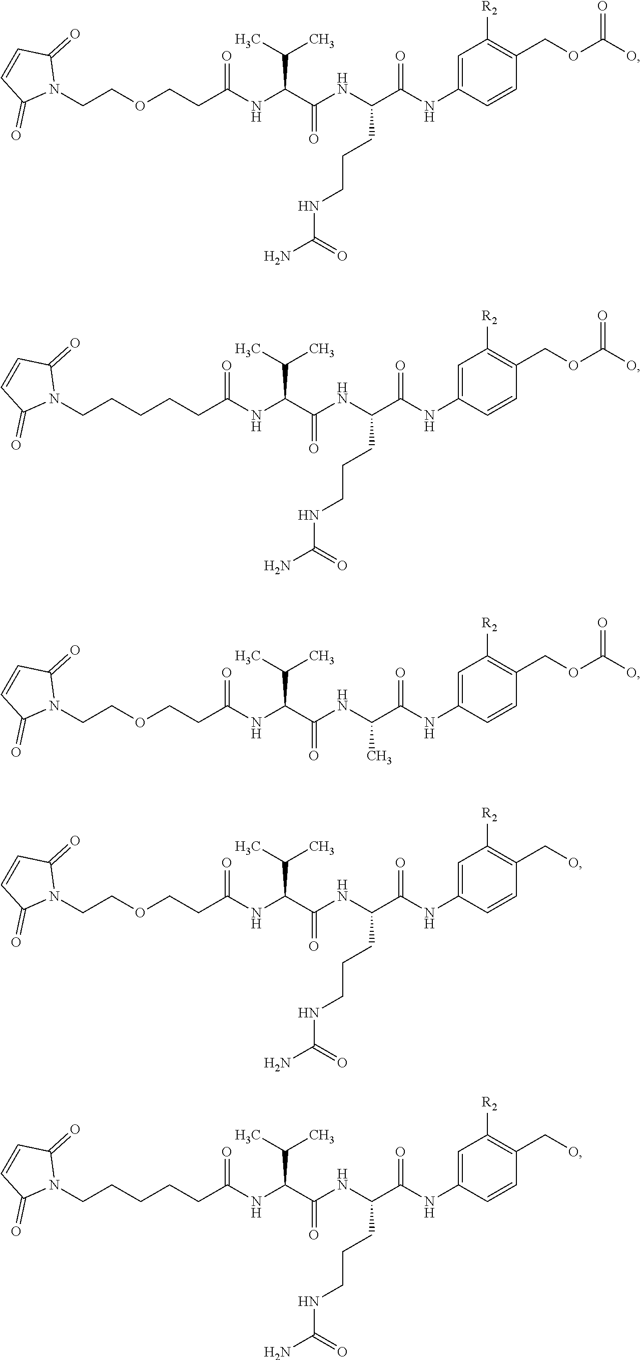

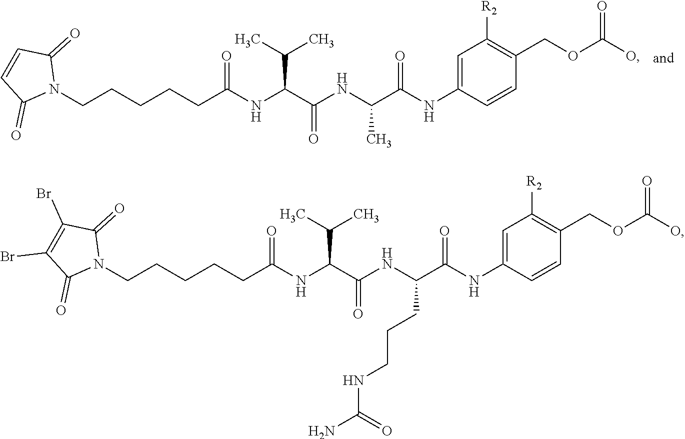

wherein R₂ and D are as defined herein before.

In a preferred embodiment, Linker-Drug compound of Formula (II) are selected from:

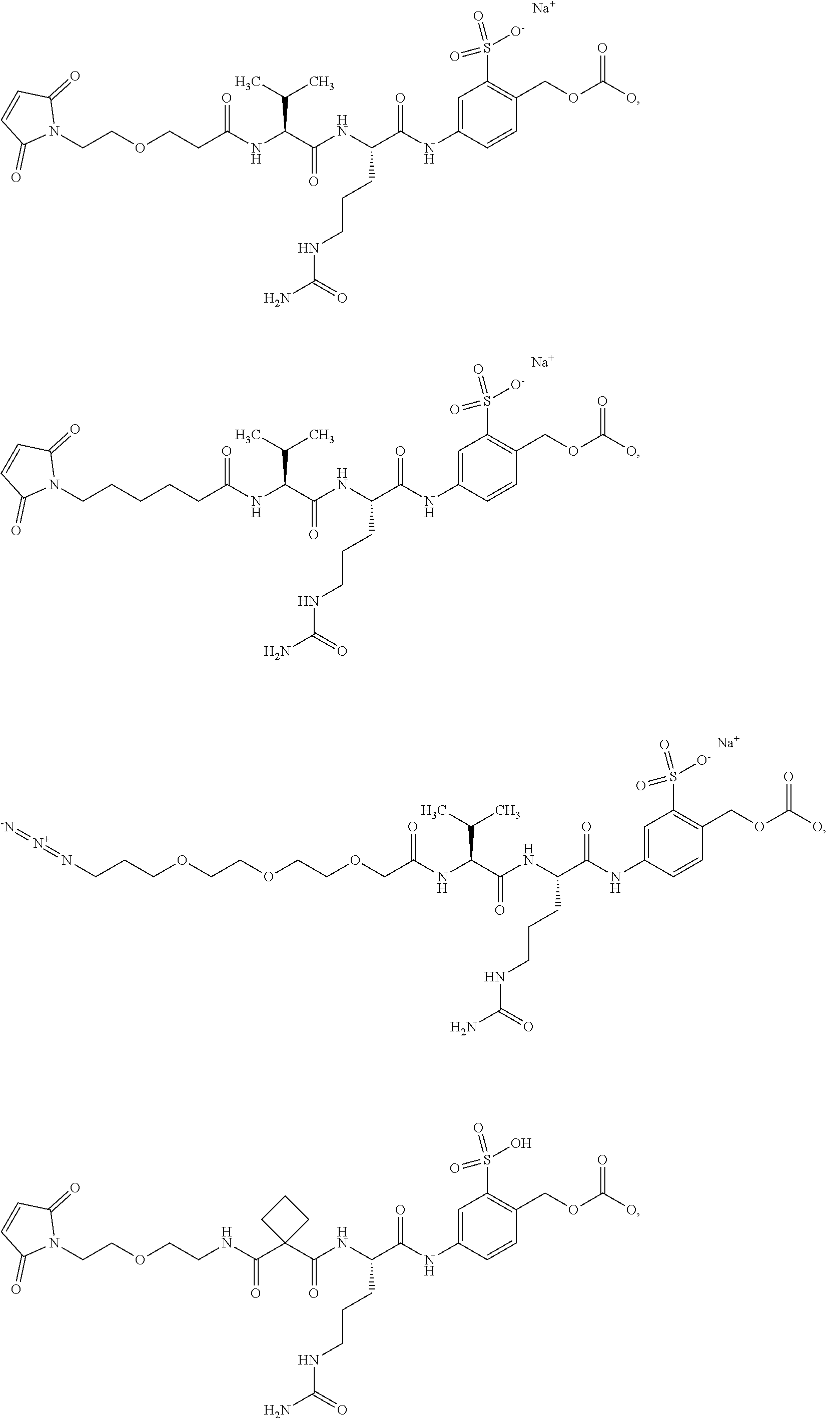

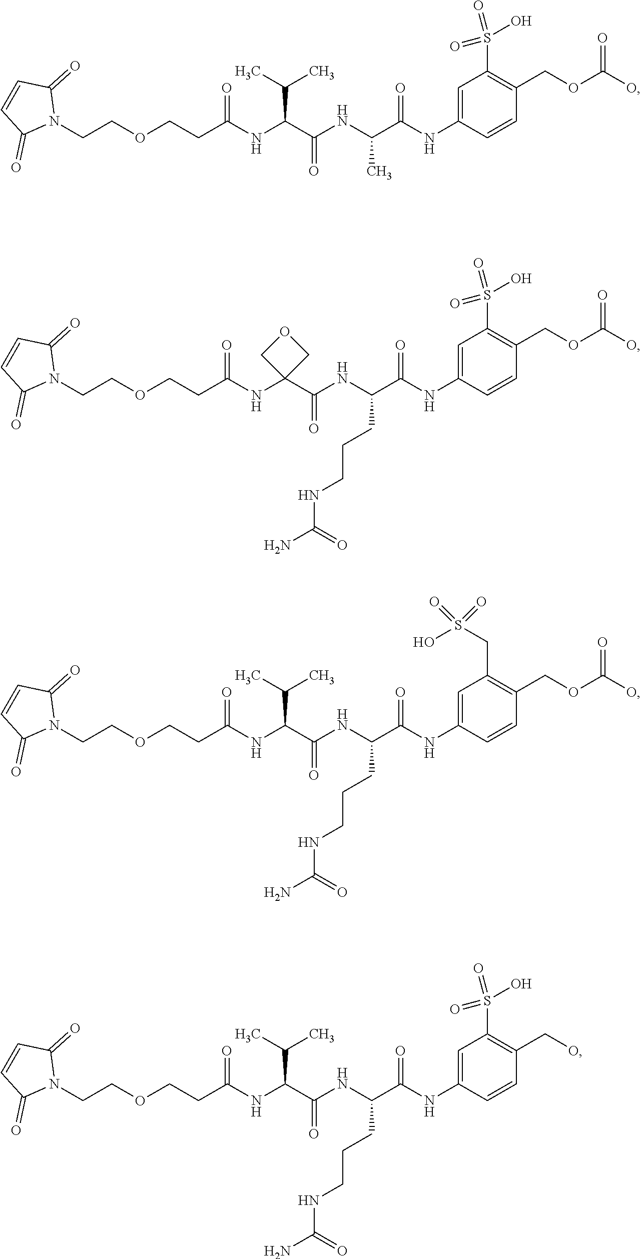

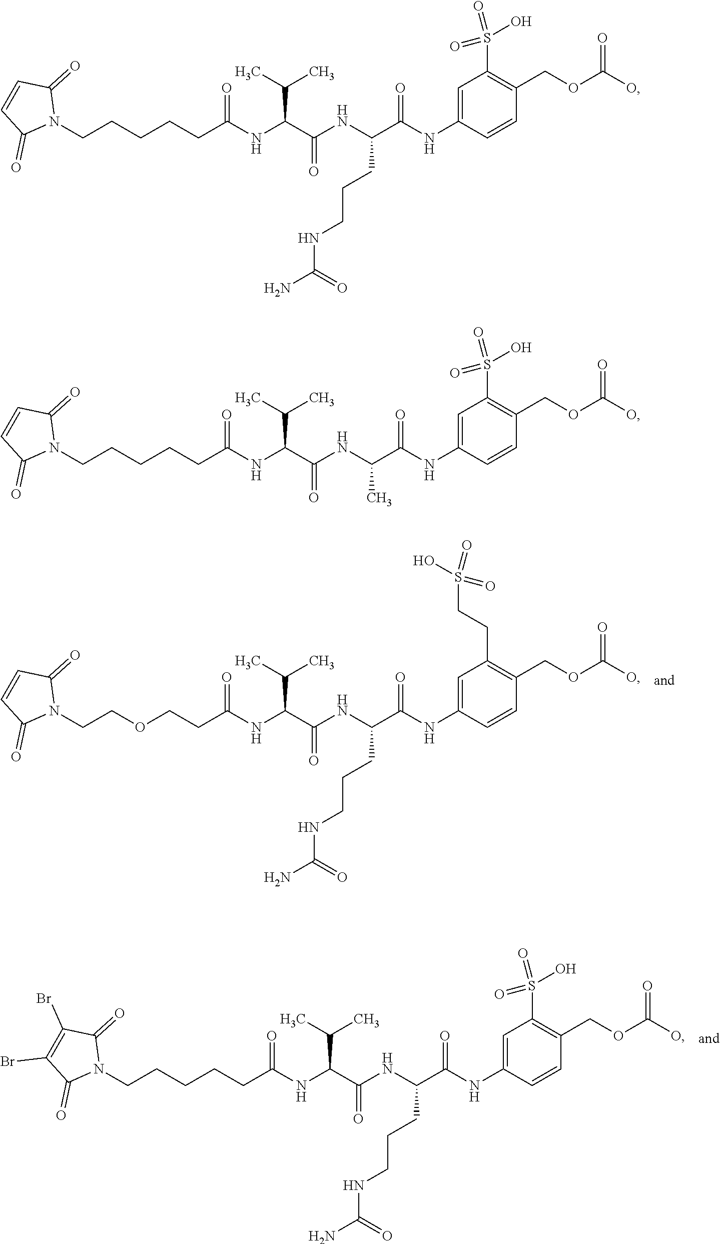

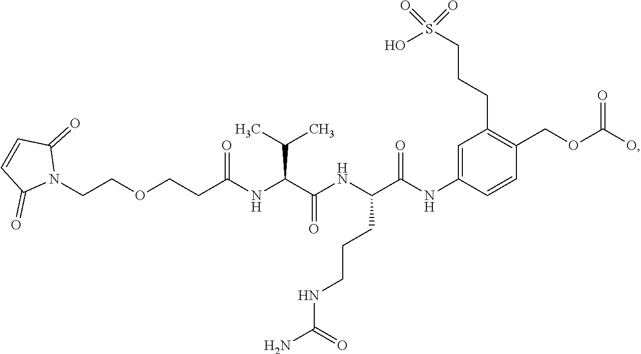

wherein D is as defined herein before.

In a particular embodiment, preferred Linker-Drug compounds of Formula (II) are:
sodium 5-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate;
sodium 5-[[(2S)-2-[[(2S)-2-[6-(2,5-dioxopyrrol-1-yl)hexanoylamino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate;
sodium 5-[[(2S)-2-[[(2S)-2-[[2-[2-[2-(2-azidoethoxy)ethoxy]ethoxy]acetyl]amino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate;
5-[[(2S)-2-[[1-[2-[2-(2,5-dioxopyrrol-1-yl)ethoxy]ethylcarbamoyl]cyclobutanecarbonyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid;
sodium 5-[[(2S)-2-[[(2S)-2-[3-[2-(2,5-dioxopyrrol-1-yl)ethoxy]propanoylamino]-3-methyl-butanoyl]amino]propanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate;
5-[[(2S)-2-[[(2S)-2-[3-[2-(2,5-dioxopyrrol-1-yl)ethoxy]propanoylamino]-3-methyl-butanoyl]amino]propanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid;
5-[[(2S)-2-[[3-[2-[2-(2,5-dioxopyrrol-1-yl)ethoxy-]ethylcarbamoyl]oxetane-3-carbonyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid;
[5-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenyl]methanesulfonic acid;
[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]-2-sulfo-phenyl]methyl-[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-dimethyl-ammonium; 2,2,2-trifluoroacetate;
N-({[4-({N-[6-(2,5-dioxo-2,5-dihydro-1H-pyrrol-1-yl)hexanoyl]-L-valyl-L-alanyl}amino)-2-sulfophenyl]methoxy}carbonyl)-N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2- methyl-3-oxopropyl]pyrrolidin-1-yl}-3-methoxy-5-methyl-1-oxoheptan-4-yl]-N- methyl-L-valinamide;
N-[6-(2,5-dioxo-25-dihydro-1H-pyrrol-1-yl)hexanoyl]-L-valyl-N⁵-carbamoyl-N-(4- {[({[(4S)-4,11-diethyl-9-hydroxy-3,14-dioxo-3,4,12,14-tetrahydro-1H-pyrano[3′,4′:6,7]indolizino[1,2-b]quinolin-4-yl]oxy}carbonyl)oxy]methyl}-3-sulfophenyl)-L-ornithinamide;
(1S,3S)-3,5,12-trihydroxy-3-(hydroxyacetyl)-10-methoxy-6,11-dioxo-1,2,3,4,6,11-hexahydrotetracen-1-yl 2,3,6-trideoxy-3-[({[4-({N-[6-(2,5-dioxo-2,5-dihydro-1H-pyrrol-1-yl)hexanoyl]-L-valyl-N⁵-carbamoyl-L-ornithyl}amino)-2-sulfophenyl]methoxy}carbonyl)amino]-α-L-lyxo-hexopyranoside;
N-{3-[2-(2,5-dioxo-2,5-dihydro-1H-pyrrol-1-yl)ethoxy]propanoyl}-L-valyl-N-{4-[(5S,8S,11S,12R)-11-[(2S)-butan-2-yl]-12-(2-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-2-oxoethyl)-4,10-dimethyl-3,6,9-trioxo-5,8-di(propan-2-yl)-2,13-dioxa-4,7,10-triazatetradecan-1-yl]-3-(2-sulfoethyl)phenyl}-N⁵carbamoyl-L-ornithinamide; and
5-[[(2S)-2-[[(2S)-2-[6-(3,4-dibromo-2,5-dioxo-pyrrol-1-yl)hexanoylamino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidon-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid.

The invention relates also to a process for the preparation of Linker-Drug compounds of Formula (II), which process is characterized in that there is used as starting material the compound of Formula (I):

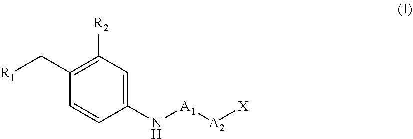

wherein R₁, R₂, A₁, A₂ and X are as defined hereinbefore
which is then modified to yield the compound of Formula (X):

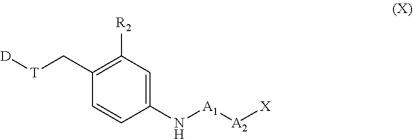

wherein A₁, A₂, D, R₂, T and X are as defined in Formula (II),
compound of Formula (X) which is further subjected, after removing the protecting group X, to coupling with the compound of Formula (XI):

O—Z′ (XI)

wherein Z′ is as defined in Formula (II),
to yield the Linker-Drug compound of Formula (II).

An alternative process for the preparation of Linker-Drug compounds of Formula (II) is characterised in that there is used as starting material the compound of Formula (VII), obtained in two steps from compound of Formula (IV), which is subjected to coupling with group D-T to yield the compound of Formula (XII):

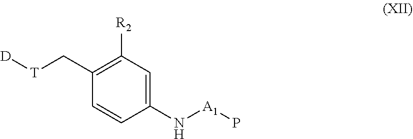

wherein A₁, D, P, R₂, and T are as defined herein before.
compound of Formula (XII) which is further subjected to coupling with the compound of Formula (XIII):

HO—A₂—Z′ (XIII)

wherein Z and A₂ are as defined in Formula (I),
to yield the Linker-Drug compound of Formula (II).

In one embodiment, Linker-Drug compound of Formula (II) obtained at the end of the above-mentioned processes may be further purified according to a conventional separation technique, which are converted, if desired, into then addition salts with a pharmaceutically acceptable acid or base and which may be separated into their isomers according to a conventional separation technique, it being understood that at any moment considered appropriate during the course of the process described above, some groups (hydroxy, amino . . . ) of the starting reagents or of the synthesis intermediates can be protected, subsequently deprotected and functionalized, as required by the synthesis. The compounds of formulae (IV), (XI) and (XIII) are either commercially available or can be obtained by the person skilled in the art using conventional chemical reactions described in the literature.

The invention relates also to the use of Linker-Drug compound of formula (II) in the preparation of an antibody-drug conjugate.

In a third aspect of the invention, there is provided an antibody-drug conjugate (ADC) of Formula (III):

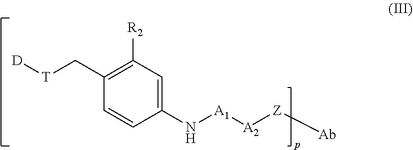

wherein
Ab represents an antibody or an antigen-binding fragment thereof;
D is a drug moiety;
T is a bond, —O—C(O)—N(CH₃)—CH₂—CH₂—N(CH₃)—C(O)—*, —O—*, —NR₅—*, —NR₅—C(O)—*, or —O—C(O)—*, wherein the * indicates the point of attachment to D;
Z represents a Spacer Unit;
A₁ represents a —C(O)—CH(R₃)—NH— group;
A₂ represents a —C(O)—CH(R₄)—NH— group, a

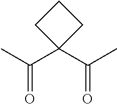

group, a

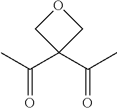

group, or a

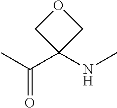

group;
R₂ represents a —S(O)₂(OH) group, a —S(O)₂(O⁻M⁺) group, a linear or branched —(C₁-C₄)alkyl-S(O)₂(OH) group, a linear or branched —(C₁-C₄)alkyl-S(O)₂O⁻M⁺) group, a linear or branched -halo(C₁-C₄)alkyl-S(O)₂(OH) group, or a linear or branched -halo(C₁-C₄)alkyl-S(O)₂(O⁻M⁺) group;
R₃ and R₄ independently of one another, represent the side chain of an amino acid;
R₅ represents a (C₁-C₄)alkyl group;
M⁺ represents a pharmaceutically acceptable monovalent cation; and
   p is an integer from 1 to 8.

In one embodiment in the third aspect of the invention, D is a drug moiety comprising a nitrogen atom or an oxygen atom, wherein D is directly connected to T via the said nitrogen atom or the oxygen atom of the drug moiety. In some embodiment in the third aspect of the invention, D is a quaternized tertiary amine-containing drug moiety. In a particular embodiment in the third aspect of the invention, D is monomethyl auristatin E (IUPAC Name: (S)-N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide or MMAE), auristatin E (IUPAC Name: (2S)-2-[[(2S)-2-(dimethylamino)-3-methyl-butanoyl]amino]-N-[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-N,3-dimethyl-butanamide), SN-38 (IUPAC Name: (4S)-4,11-diethyl-4,9-dihydroxy-1H-pyrano[3′,4′:6,7]indolizino[1,2-b]quinoline-3,14(4H,12H)-dione), or doxorubicin (IUPAC Name: (1S,3S)-3,5,12-trihydroxy-3-(hydroxyacetyl)-10-methoxy-6,11-dioxo-1,2,3,4,6,11-hexahydrotetracen-1-yl 3-amino-2,3,6-trideoxy-α-L-lyxo-hexopyranoside).

Preferably in the third aspect of the invention, T is a bond or —O—C(O)—*, wherein the * indicates the point of attachment to D. In some embodiment in the third aspect of the invention, when T is a bond, D is a quaternized tertiary amine-containing drug moiety.

Preferably in the third aspect of the invention, R₂ represents a —S(O)₂(OH) group, a —S(O)₂(O⁻M⁺), a —CH₂—S(O)₂(OH) group, a —CH₂S(O)₂(O⁻M⁺) group, a —CH₂—CH₂—S(O)₂(OH) group, a —CH₂—CH₂—S(O)₂O⁻M⁺) group, a —CH₂—CH₂—CH₂—S(O)₂(OH) group, or a —CH₂—CH₂—CH₂—S(O)₂(O⁻M⁺) group. In a preferred embodiment of the third aspect of the invention, M⁺ represents Na⁺.

Advantageously in the third aspect of the invention, A₁ represents a —C(O)—CH(R₃)—NH— group in which R₃ represents a —(CH₂)₃—NH—CO—NH₂ group or a methyl group.

In a preferred embodiment of the third aspect of the invention, A₂ represents a —C(O)—CH(R₄)—NH— group in which R₄ represents an isopropyl group; a

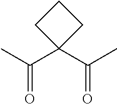

group; a

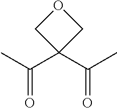

group; or a

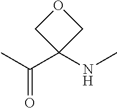

group.

In another preferred embodiment of the third aspect of the invention, A₁ represents a —C(O)—CH(R₃)—NH— group in which R₃ represents a —(CH₂)₃—NH—CO—NH₂ group, and A₂ represents a

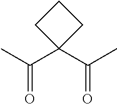

group, or a

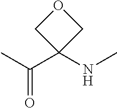

group.

In another preferred embodiment of the third aspect of the invention, A₁ represents a —C(O)—CH(R₃)—NH— group and A₂ represents a —C(O)—CH(R₄—NH— group, in which R₃ and R₄, independently of one another, represent the side chain of an amino acid. Particularly, when R₃ and R₄ represent a side chain of an amino acid, the side chain-bearing carbon is L configuration. In a preferred embodiment, A₁ represents a —C(O)—CH(R₃)—NH— group in which R₃ represents a —(CH₂)₃—NH—CO—NH₂ group or a methyl group and A₂ represents a —C(O)—CH(R₄)—NH— group in which R₄ represents an isopropyl group. Preferably in the third aspect of the invention, A₁-A₂ group represents Cit-Val or Ala-Val.

In a preferred embodiment, an ADC of Formula (III) is formed from a Linker-Drug compound of Formula (II) selected from:

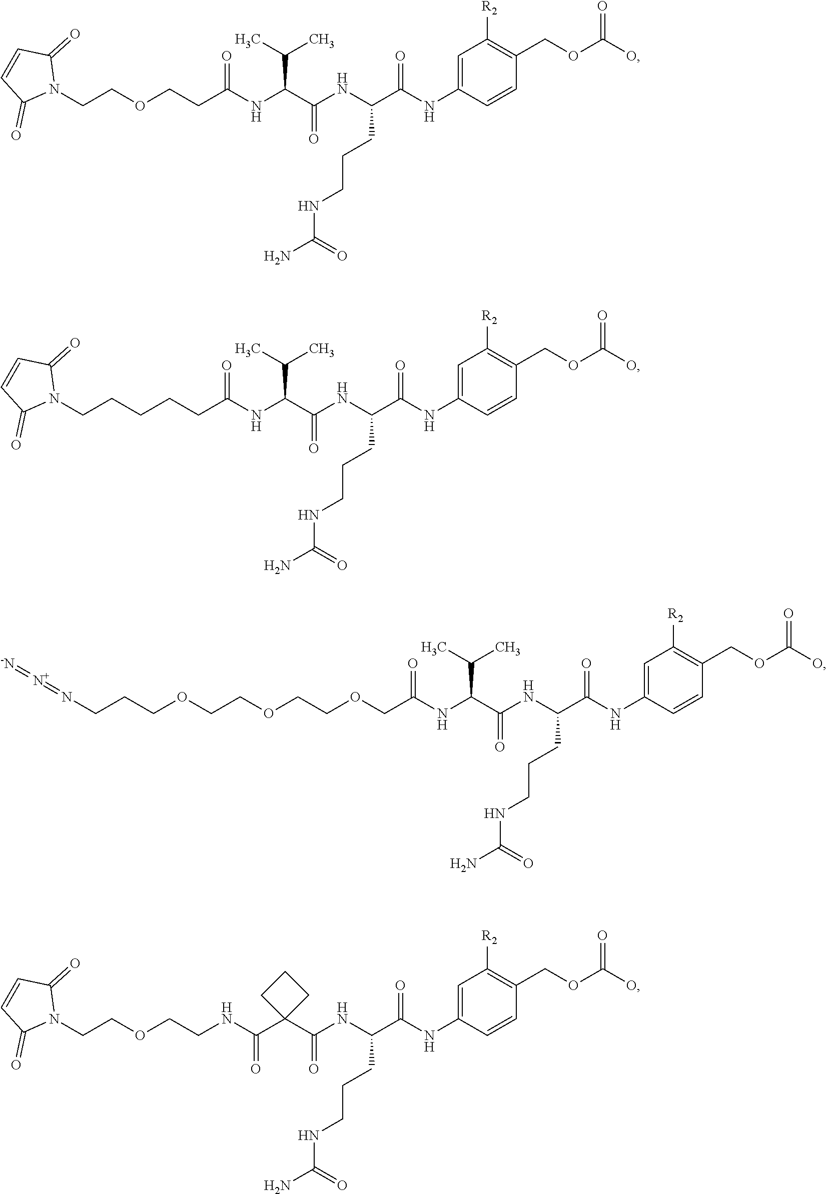

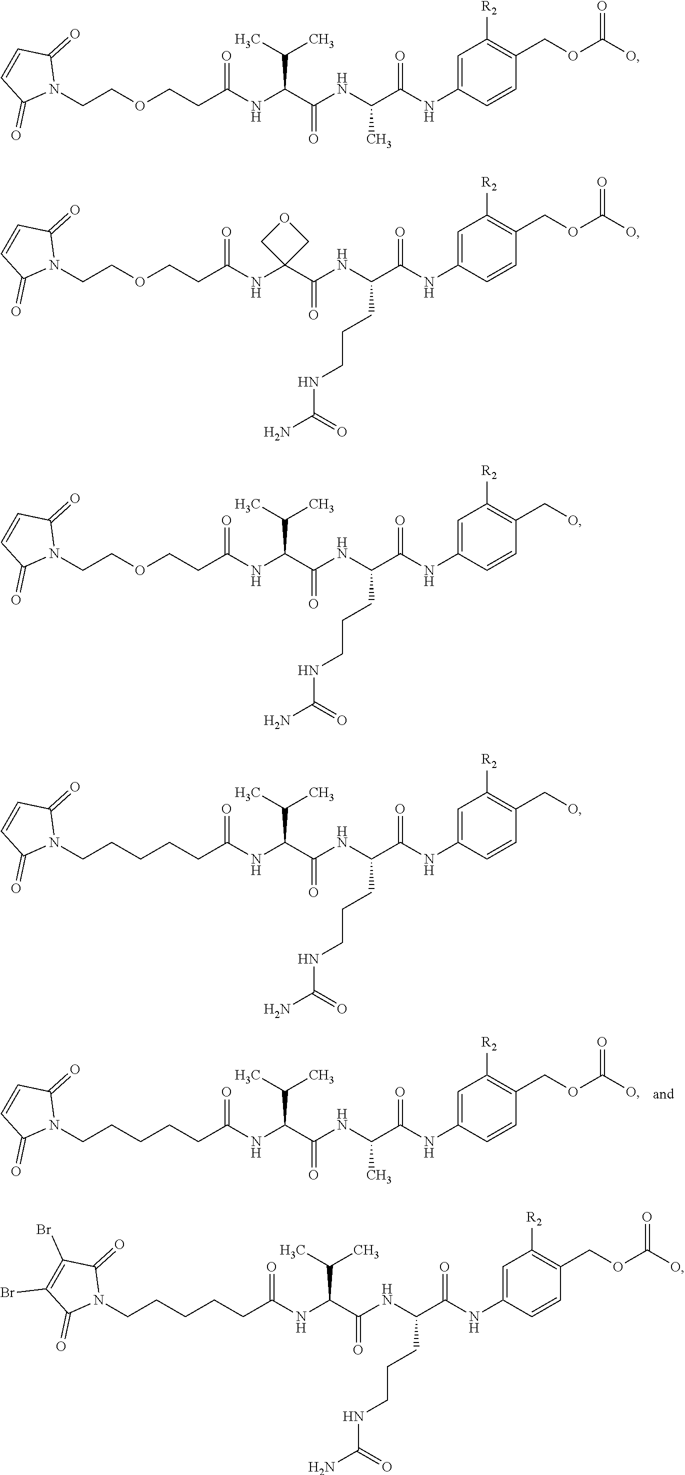

wherein R₂ and D are as defined herein before.

More preferably, an ADC of Formula (III) is formed from a Linker-Drug compound of Formula (II) selected from:

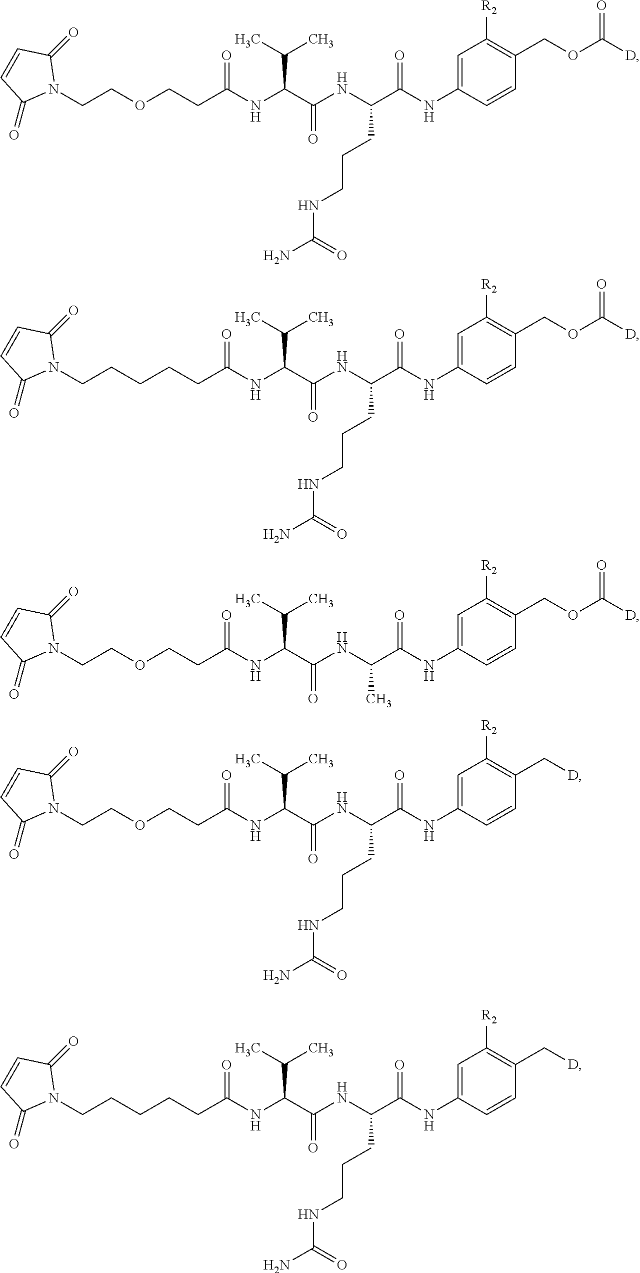

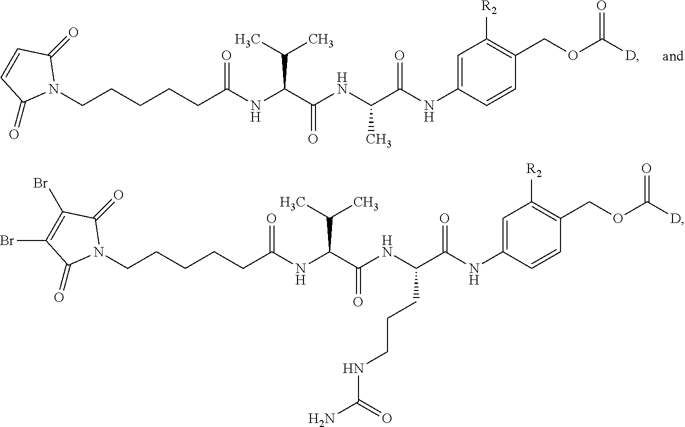

wherein R₂ and D are as defined herein before.

In a preferred embodiment, an ADC of Formula (III) is formed from a Linker-Drug compound of formula (II) selected from:

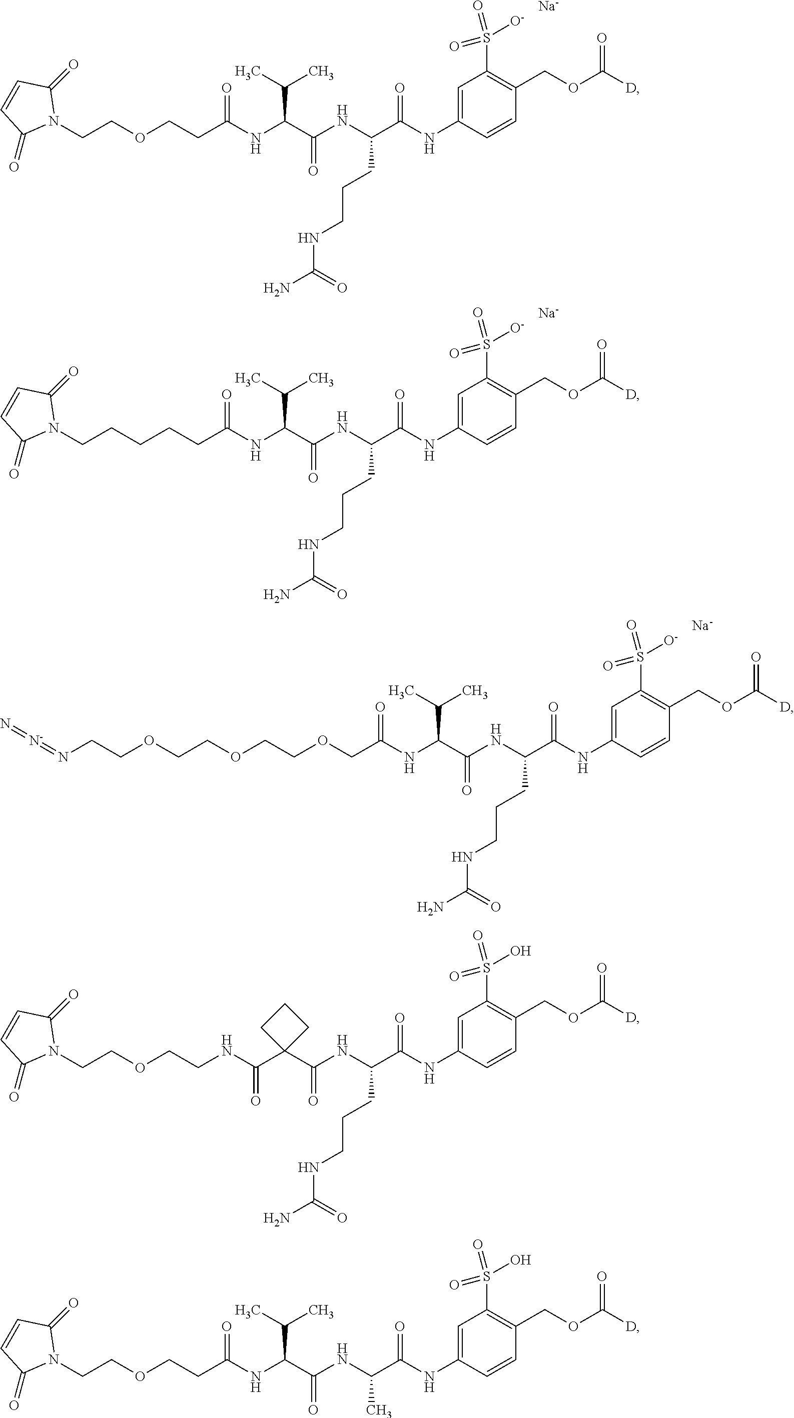

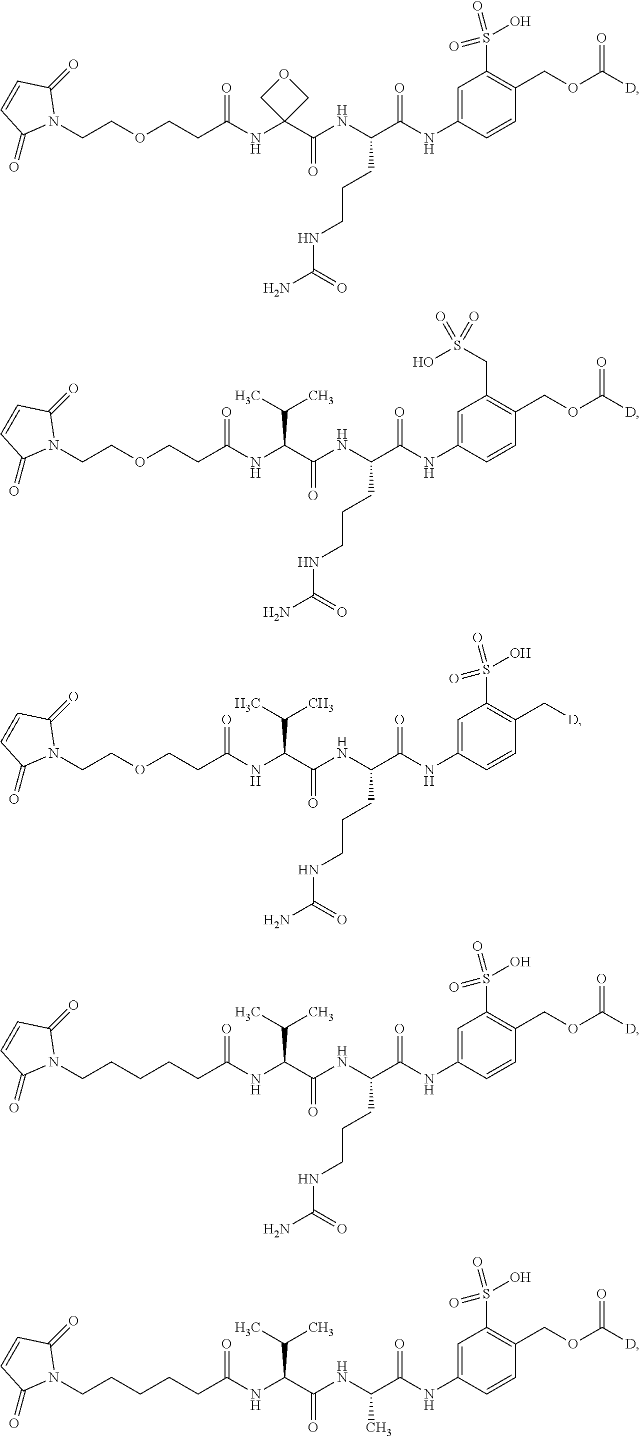

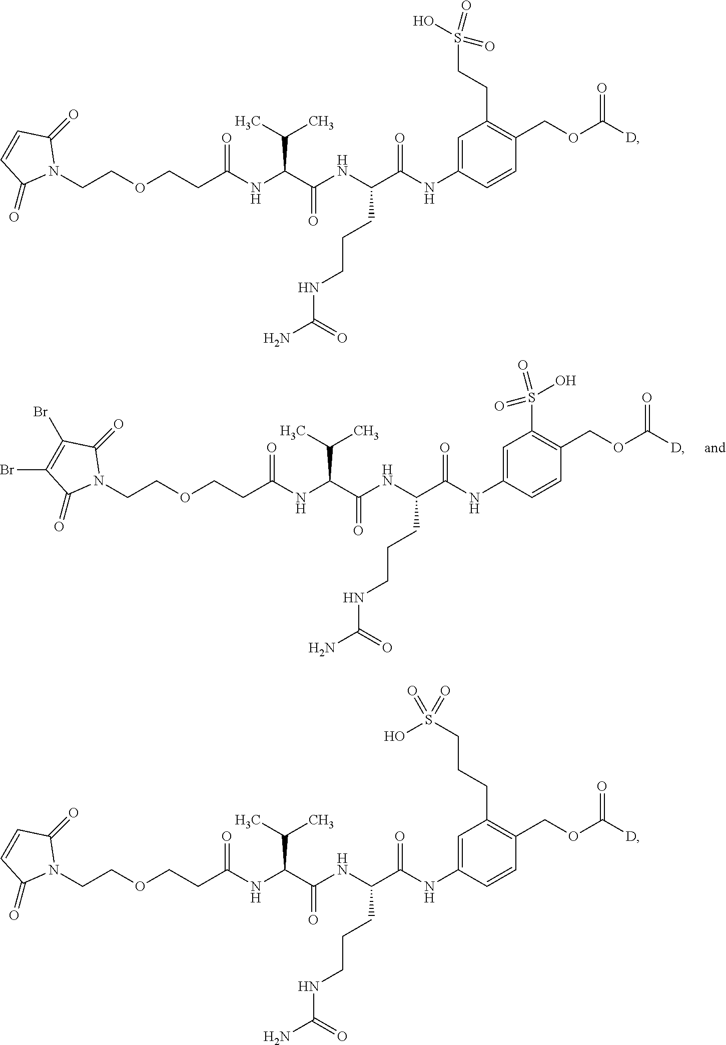

wherein D is as defined herein before.

In a particular embodiment, the invention provides an antibody-drug conjugate of Formula (III) wherein

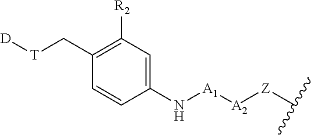

comprises a formula selected from:

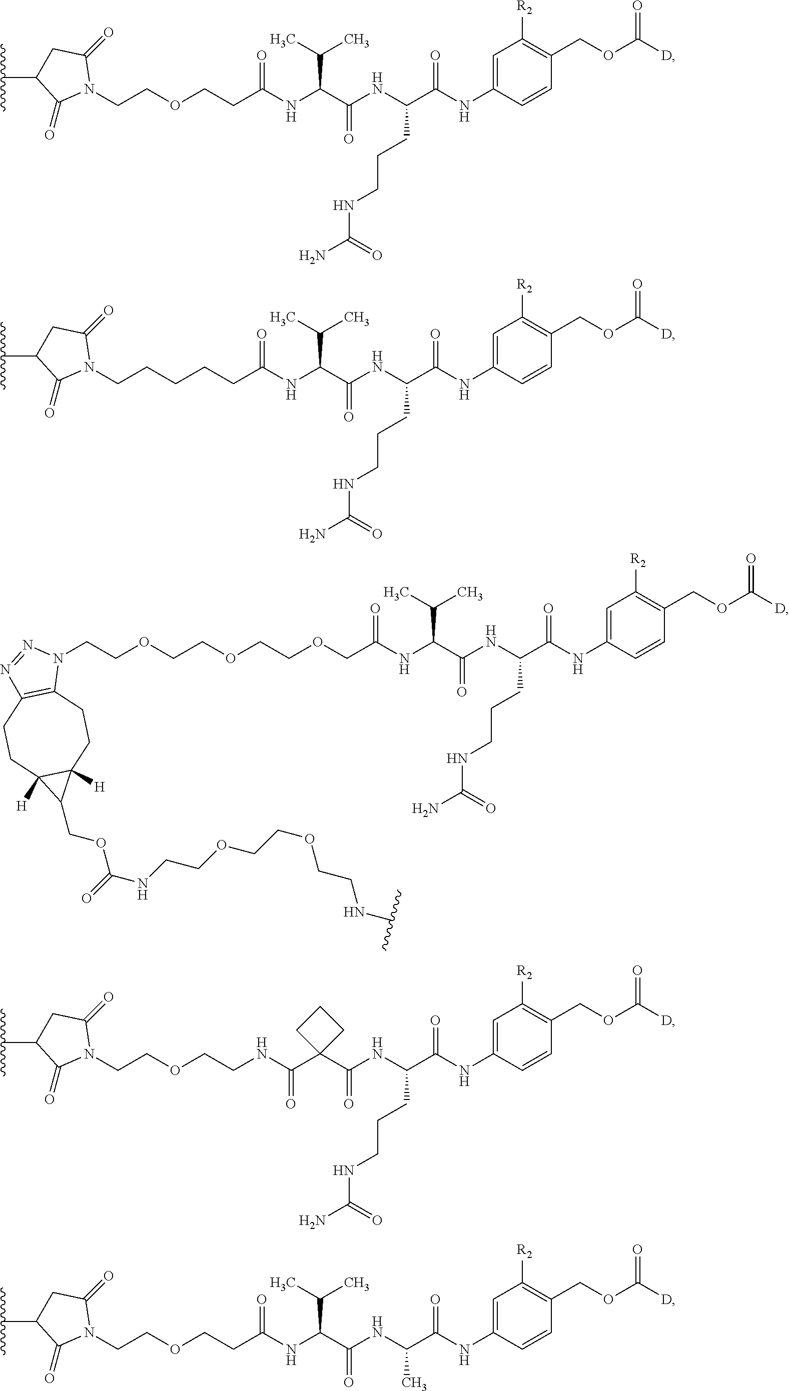

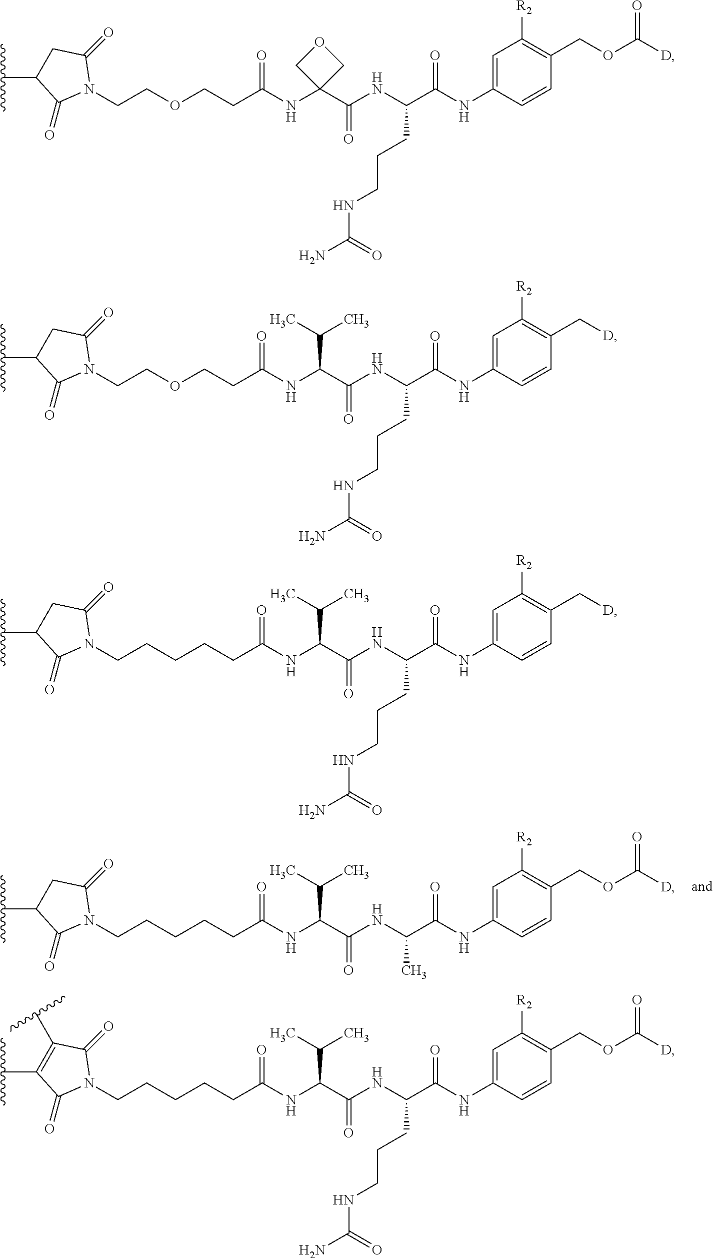

wherein wavy line indicates the covalent attachment site to the antibody and, R₂ and D are as defined herein before.

In a preferred embodiment, the invention provides an antibody-drug conjugate of Formula (III) wherein

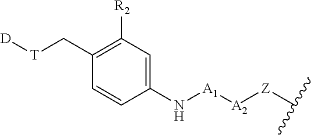

comprises a formula selected from:

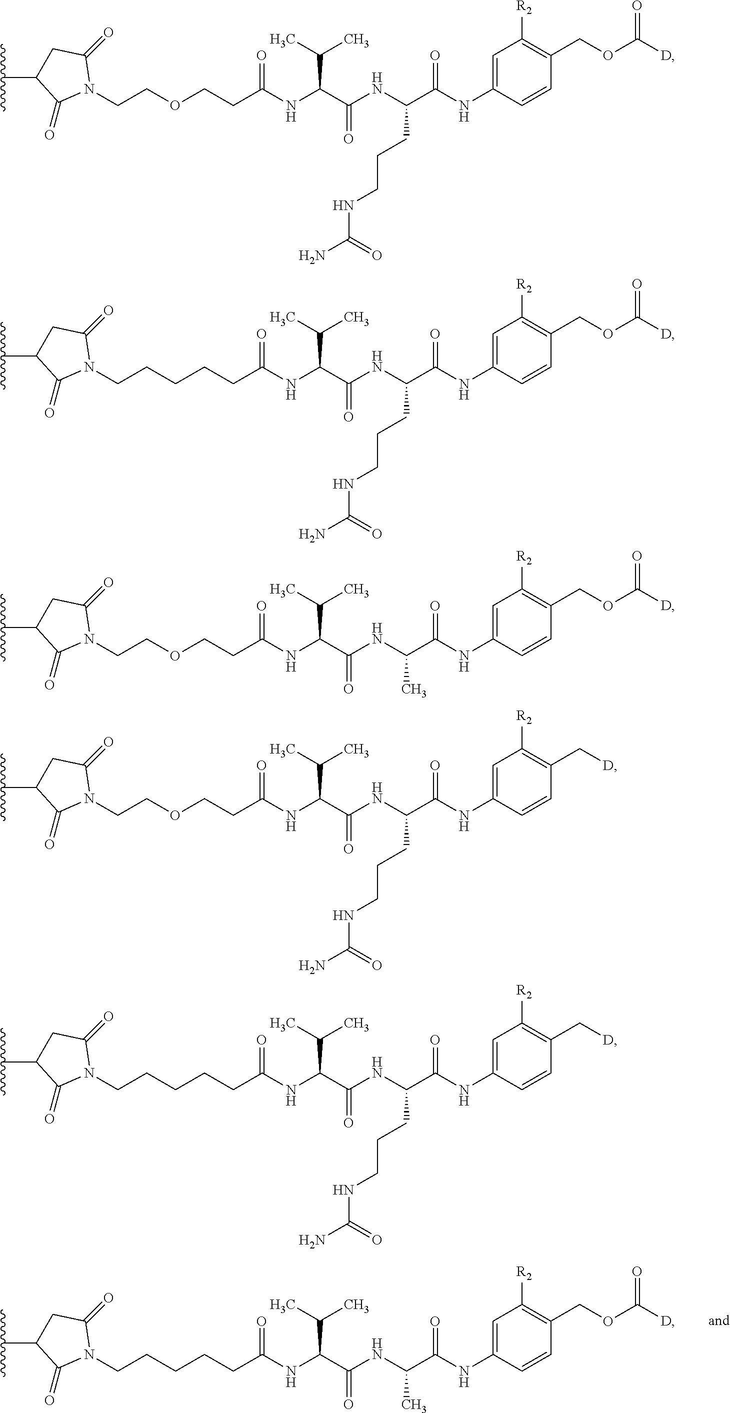

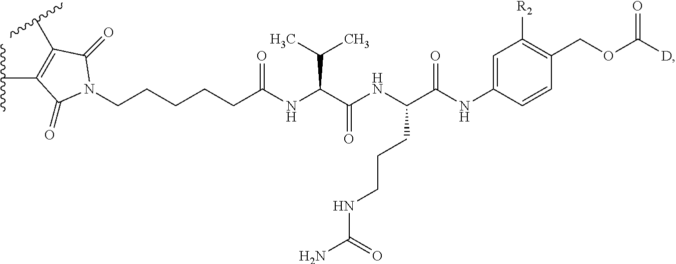

wherein wavy line indicates the covalent attachment site to the antibody and, R₂ and D are as defined herein before.

In a preferred embodiment, the invention provides an antibody-drug conjugate of Formula (III) wherein

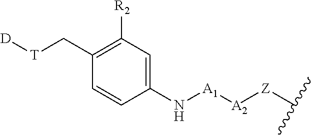

comprises a formula selected from:

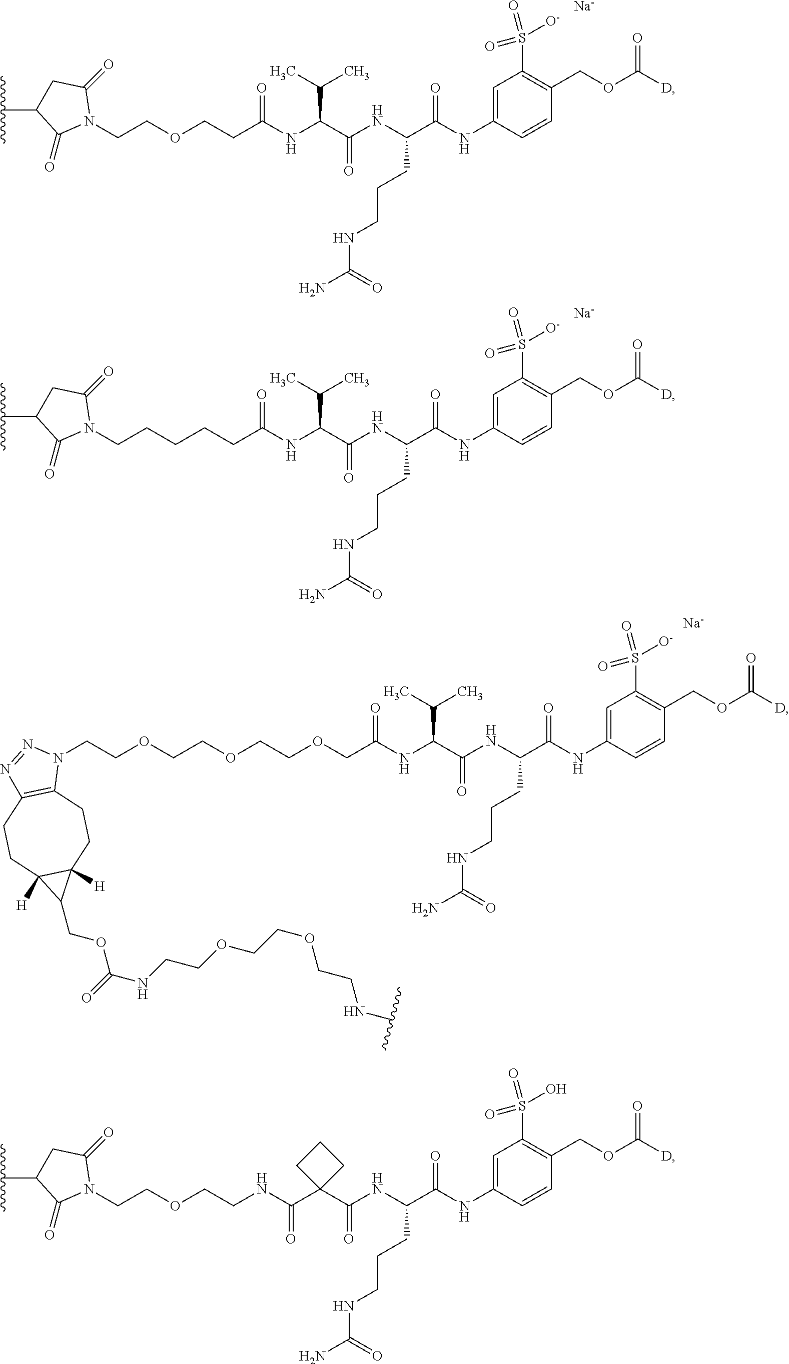

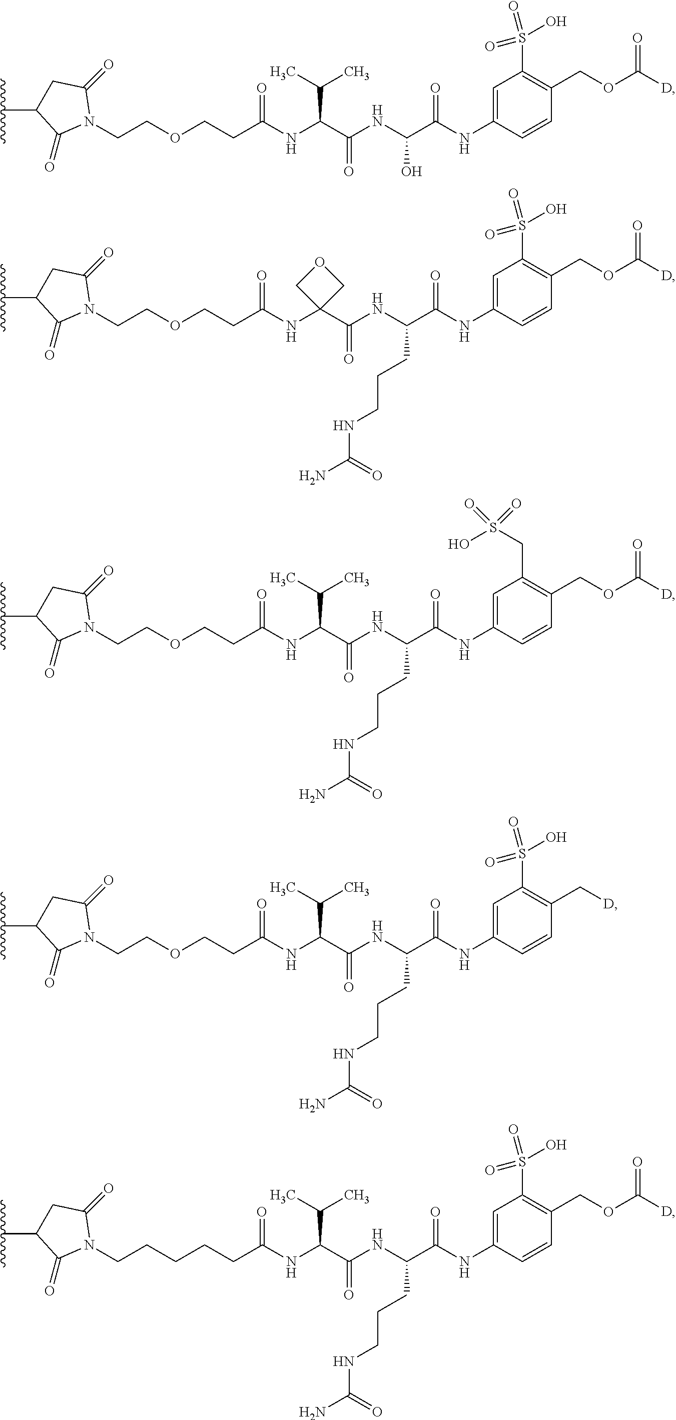

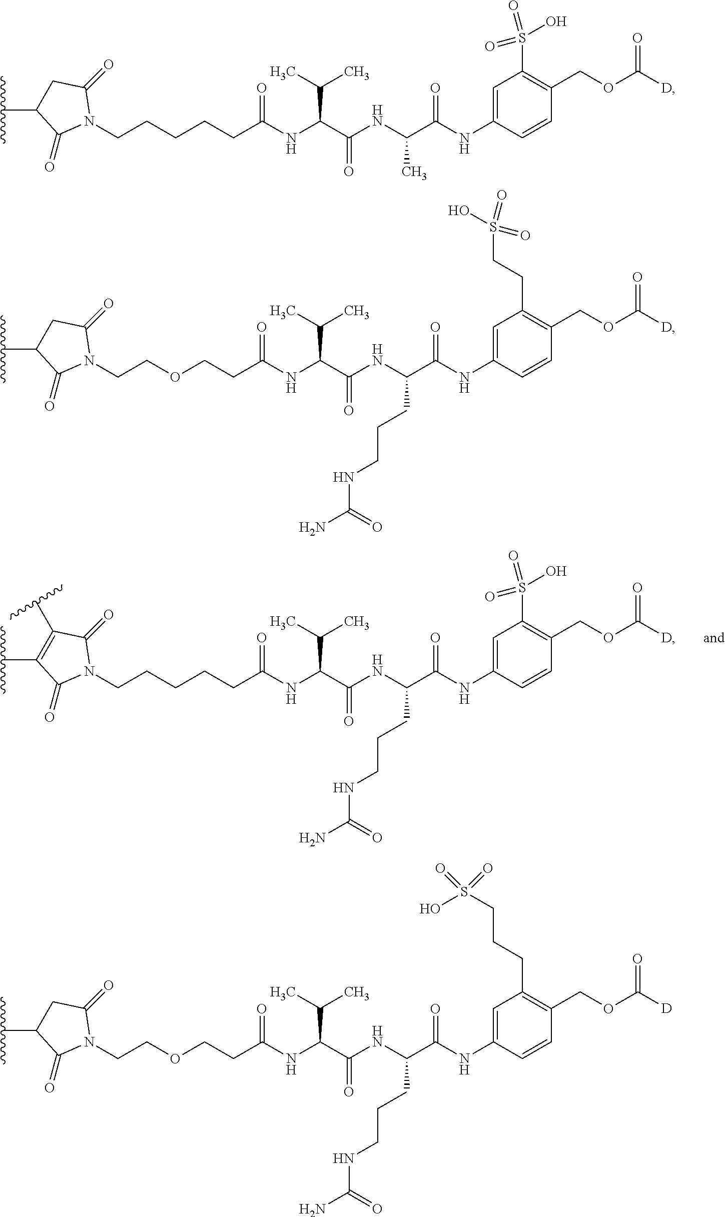

wherein wavy line indicates the covalent attachment site to the antibody and D is as defined herein before.

The ADC according to the invention are useful for treating diseases in mammal, e.g. a patient, in need thereof. The ADC can be used accordingly in a variety of settings for the treatment of a disorder associated with expression of the antigen to which the antibody of the ADC binds, e.g. cancer. The ADC can be used to deliver a drug to a targeted cell. Without being bound by theory, in one embodiment, the antibody of an ADC binds to or associates with a cell-surface antigen or receptor and upon binding the ADC can be taken up (internalized) inside said cell through antigen- or receptor-mediated endocytosis or other internalization mechanism. The antigen can be attached to the said cell or can be an extracellular matrix protein associated with the said cell. Once inside the cell, via an enzymatic or non-enzymatic cleavable mechanism, depending upon the components of the linker system, the drug is released within the cell, in an alternative embodiment, the drug is cleaved from the ADC within the vicinity of the said cell, and the drug subsequently penetrates the cell.

The present invention provides pharmaceutical compositions comprising an ADC composition described herein and a pharmaceutically acceptable carrier. The pharmaceutical compositions can be in any form that allows for an ADC to be administered to a patient for treatment of a disorder associated with expression of the antigen to which the antibody of the ADC binds. Particularly, the present invention relates to pharmaceutical compositions comprising at least one antibody-drug conjugate of Formula (III) in combination with one or more pharmaceutically acceptable excipients. For example, the pharmaceutical compositions can be in the form of a liquid or a lyophilized solid. Among the pharmaceutical compositions according to the invention there may be mentioned more especially those that are suitable for oral, parenteral, nasal, per- or trans-cutaneous, rectal, perlingual, ocular or respiratory administration. The preferred route of administration is parenteral. Parenteral administration includes subcutaneous injections, intravenous, intramuscular, and intrasternal injection or infusion techniques. In preferred embodiments, a pharmaceutical composition comprising an ADC is administered intravenously in the form of a liquid solution. The liquid can be useful for delivery by injection. In a composition for administration by injection, one or more of a surfactant, preservative, wetting agent, dispersing agent, suspending agent, buffer, stabilizer, diluents, lubricants, binders, disintegration agents, absorbents and isotonic agent can also be included.

In another aspect, the present invention relates to the use of any of the antibody-drug conjugates defined above for the manufacture of a pharmaceutical preparation for the treatment of a mammal being in need thereof. In another aspect, the present invention relates to any of the antibody-drug conjugates defined above for use in the treatment of a mammal being in need thereof. The invention also relates to methods of treating a mammal being in need thereof whereby the method comprises the administration of a pharmaceutical composition to the mammal in a therapeutically effective dose.

The invention is further exemplified by the following Examples. These examples are for illustrative purposes and are not intended to limit the scope of the invention.

### Abbreviations

ACN acetonitrile (or CH₃CN)
BBS borate buffered saline
DBCO dibenzocyclooctyne

### DEA N,N-diethylamine

DCM dichloremethane
DIEA diisopropylethylamine

### DIPEA N,N-diisopropylethylamine

DMA dimethylacetamide
DMAP 4-dimethylaminopyridine
DMF dimethylformamide (or N,N-dimethylformadide)
DMSO dimethylsulfoxide
EDC N-ethyl-N′,N′-dimethylamino-propylcarbodiimide
EDTA ethylenediaminetetraacetic acid
EEDQ N-Ethoxycarbonyl-2-ethoxy-1,2-dihydroquinoline
EtOAc ethyl acetate
EtOH ethanol
FA formic acid
HATU 1-[bis(dimethylamino)methylene]-1H-1,2,3-triazolo[4,5-b]pyridinium 3-oxide hexafluorophosphate
HBTU [benzotriazol-1-yloxy(dimethylamino)methylene]-dimethyl-ammonium; hexafluorophosphate
HOAt 1-hydroxy-7-azabenzotriazole
HOBt 1-hydroxy-benzotriazole
MeOH methanol
MMAE (2S)-N-[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]-3-methyl-2-(methylamino)butanamide
NMP N-methyl-2-pyrrolidone
PBS phosphate-buffered saline
Pd/C Palladium on carbon
rmp multi-point attachment of Protein A
r.t. room temperature
TEA triethylamine
TEA trifluoroacetic acid
THF tetrahydrofuran
TSTU [dimethylamino-(2,5-dioxopyrrolidin-1-yl)oxy-methylene]-dimethyl-ammonium; tetrafluoroborate

Materials and Methods/General Procedures

All reagents obtained from commercial sources were used without further purification. Anhydrous solvents were obtained from commercial sources and used without further drying. Flash chromatography was performed on Combi flash Rf (Teledyne ISCO) with pre-packed silica-gel cartridges (Macherey-Nagel Chromabond Flash). Thin layer chromatography was conducted with 5×10 cm plates coated with Merck Type 60 F254 silica-gel. Microwave heating was performed in CEM Discover® instrument.

¹H-NMR measurements were performed on 400 MHz Broker Avance or 500 MHz Avance Neo spectrometer, using DMSO-d6 (also written as dmso-d6 or DMSO) or CDCl₃ as solvent. ¹H NMR data is in the form of delta values, given in pan per million (ppm), using the residual peak of the solvent (2.50 ppm for DMSO-d6 and 7.26 ppm for CDCl₃) as internal standard. Splitting patterns are designated as: s (singlet), d (doublet), t (triplet), q (quartet), m (multiplet), brs (broad singlet), dd (doublet of doublets), brm (broad multiplet), td (triplet of doublets), dt (doublet of triplets), br dd (broad doublet of doublets).

IR measurements were performed on a Bruker Tensor 27 equipped with ATR Golden Gate device (SPECAC). HRMS measurements were performed on a LTQ OrbiTrap Velos Pro mass spectrometer (Thermofisher Scientific). Samples were dissolved in CH₃CN/H₂O (2/1:v/v) at a concentration range from 0.01 to 0.05 mg/mL approximately and introduced in the source by an injection of 2 μL in a flow of 0.1 mL/min.

ESI ionization parameters were as follows: 3.5 k-V and 350° C. transfer ion capillary. All the spectra were acquired in positive ion mode with a resolving power of 30 000 or 60 000 using a lock mass.

UPLC-MS:

Waters Aquity A-class with diode array UV detector “PDA” and “ZQ detector 2” mass device and MassLinks software

ZQ detector 2: MS scan from 0.15 to 6 min and from 100 to 2372 Da

PDA detector: from 190 to 400 nm

Column: Acquity UPLC® BEH column C₁₈, 1.7 μm, 130 Å, 2.1×50 mm

Column used at 40° C. with a flow rate of 0.6 mL/min

Solvent A: water+0.02% TFA, Solvent B: acetonitrile+0.02% TEA

Gradient from 2% B to 100% B in 5 minutes, then 0.3 minutes washing with 100% B and 0.5 minutes equilibration at 2% B for the next injection (total gradient of 6 minutes)

Prep-HPLC:

Interchim Puriflash 4100® with a maximum of 100 bars and a maximum flow rate of 250 mL/min or Interchim Puriflash 4250® with a maximum of 250 bars and a maximum flow rate of 250 mL/min.

Quaternary solvent pump with the possibility to use 4 solvents at the same time in a gradient UV: 2 wavelengths for the collection between 200 and 400 nm

Collection: 8 mL or 32 mL tubes

Columns Waters Xbridge® 10 μm

3 Frep-HPLC methods were used:
1) TEA method: solvent: A=water+0.05% TFA, B=acetonitrile+0.05% TFA, gradient from 5 to 100% B in 15 to 30 CV (Column Volume)
2) NH₄HCO₃ method: solvent: A=water+0.02 M NH₄HCO₃, B=acetonitrile/water 80/20+0.02M NH₄HCO₃, gradient from 5 to 100% B in 15 to 30 CV
3) Neutral method: solvent: A=water, B=acetonitrile, gradient from 5 to 100 % B in 15 to 30 CV

All the fractions containing the pure compound were combined and directly freeze-dried to afford the compound as an amorphous powder.

Preparative SFC Purification:

Preparative chiral SFC was performed on a PIC solution Prep200 system. The sample was dissolved in ethanol at a concentration of 150 mg/mL. The mobile phase was held isocratically at 40% ethanol/CO₂. The instrument was fitted with a Chiralpak IA column and a loop of 3 mL. The ABPR (automatic back-pressure regulator) was set at 100 bars.

IUPAC chemical names were generated using Biovia Draw Version 18.1.NET software or using ACD/Name 2018.2.2 (File Version N50E41, Build 103230, 21 Jul. 2018) software.

Preparation 1: (2,5-dioxopyrrolidin-1-yl) 1-[2-[2-(2,5-dioxopyrrol-1-yl)ethoxy]ethylcarbamoyl]cyclobutanecarboxylate

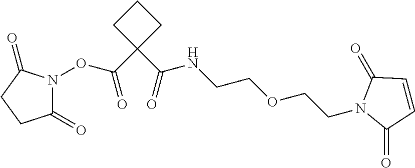 [SMILES: O=C1C=CC(=O)N1CCOCCNC(=O)C1(C(=O)ON2C(=O)CCC2=O)CCC1]

Step 1: tert-butyl 1-[2-[2-(2,5-dioxopyrrol-1-yl)ethoxy]ethylcarbamoyl]cyclobutanecarboxylate

To a solution of 1-tert-butoxycarbonylcyclobutanecarboxylic acid (58.6 mg; 0.293 mmol) in OCM (5.85 mL), were successively added 1-[2-(2-aminoethoxy)ethyl]pyrrole-2,5-dione (53.9 mg; 0.293 mmol), EDC (84.2 mg; 0. 439 mmol). HOBt (59.3 mg, 0.439 mmol) and DIPEA (204 μL; 1.17 mmol). The reaction mixture was stirred at r.t. for 18 hours and then was concentrated to dryness and solubilized in DMF (1 mL). After solubilization of the residue in DMF (1 mL), the crude was purified by C18 reverse phase prep-HVLC by direct deposit of the reaction mixture on the Xbridge® column and using the TFA method to afford the title compound (57.3 mg, 0.156 mmol). IR (cm⁻¹): 3390, 1697/1666. ¹H NMR (400 MHz, dmso-d6) δ ppm 7.5 (t, 1H), 7.02 (s, 2H), 3.55/3.5 (2t, 4H), 3.38 (t, 2H), 3.17 (q, 2H), 2.33 (m, 4H), 1.77 (m, 2H), 1.38 (s, 9H). UPLC-MS: MS(ESI): m/z [M+Na]+=389.26, [M+H−tBu]+=311.11

Step 2: 1-[2-[2-(2,5-dioxopyrrol-1-yl)ethoxy]ethylcarbamoyl]cyclobutanecarboxylic acid

To a solution of tert 1-[2-[2-(2,5-dioxopyrrol-1-yl)ethoxy]ethylcarbamoyl]cyclobutanecarboxylic acid (7 mg; 0.0191 mmol) in DCM (0.175 mL), was added TFA (51.2 μL; 0.668 mmol). The reaction mixture was stirred at r.t. for 3.5 hours, then was concentrated to dryness to obtain the title compound (5.8 mg; 0.0187 mmol) as a colorless oil. The crude product was used in the next step UPLC-MS: MS(ESI): m/z [M+H]+=3.11.35, [M+Na]+=333.37

Step 3: Preparation 1

To a solution of 1-[2-[2-(2,5-dioxopyrrol-1-yl)ethoxy]ethylcarbamoyl]cyclobutanecarboxylic acid (8.47 mg; 0.0273 mmol) in DMF (0.560 mL), were successively added TSTU (9.04 mg, 0.030 mmol) and DIPEA (9.5 μL; 0.0540 mmol). The reaction mixture was stirred at r.t. for 2 hours to afford a solution of Preparation 1 in DMF. The crude product will be used directly for next steps. UPLC-MS: MS(ESI); m/z [M+H]+=408.43, [M+Na]+=430.38

Preparation 2: (2,5-dioxopyrrolidin-1-yl) 3-[3-[2-(2,5-dioxopyrrol-1-yl)ethoxy]propanoylamino]oxetane-3-carboxylate

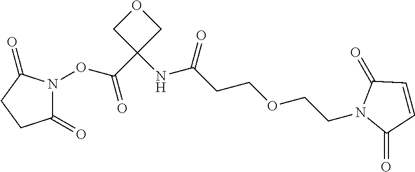 [SMILES: O=C(CCOCCN1C(=O)C=CC1=O)NC1(C(=O)ON2C(=O)CCC2=O)COC1]

Step 1 synthesis of 3-[3-[2-(2,5-dioxopyrrol-1-yl)ethoxy]propanoylamino]oxetane-3-carboxylic acid

To a solution of 3-aminooxetane-3-carboxylic acid (115 mg; 0.982 mmol) in DMF (3 45 mL), were successively added (2,5-dioxopyrrolidin-1-yl) 3-[2-(2,5-dioxopyrrol-1-yl)ethoxy]propanoate (274.3 mg; 0.883 mmol) and DIPEA (855 μL; 4.91 mmol). The reaction mixture was stirred at r.t. for 18 hours, then was concentrated to dryness and solubilized in DMF (1 mL). The crude product solution was purified by C₁₈ reverse phase prep-HPLC by direct deposit of the reaction mixture on the Xbridge® column and using the TFA method to afford the title compound (16 mg; 0.0512 mmol). UPLC-MS: MS(ESI): m/z [M+H]+=313.09 [M+Na]+=335.06. ¹H NMR (400 MHz, dmso-d6) δ ppm 8.92 (s, 1H), 7.02 (s, 2H), 4.8/4.45 (2d, 4H), 3.57/3.48 (2m, 6H), 2.32 (t, 2H), IR (cm⁻¹): 3700-2300, 1769/1740/1697, 692

Step 2: Preparation 2

To a solution of 3-[2-[2-(2,5-dioxopyrrol-1-yl)ethoxy]ethylcarbamoyl]oxetane-3-carboxylic acid (5.8 mg; 0.0187 mmol) in DMF (0.380 mL), were successively added TSTU (6.32 mg; 0.0210 mmol) and DIPEA (6.7 μL; 0.0382 mmol). The reaction mixture was stirred at r.t. for 2 hours to afford a solution of Preparation 2 in DMF. The crude product will be used directly for next steps. UPLC-MS: MS(ESI): m/z [M+H]+=310.29, [M+Na]+=332.27

### EXAMPLE 1

### sodium 5-[[(2S)-2-[[(2S)-2-(9H-fluoren-9-ylmethoxycarbonylamino)-3-methyl-butanoyl]amino]-5-ueido-pentanoyl]amino]-2-(hydroxymethyl)benzenesulfonate

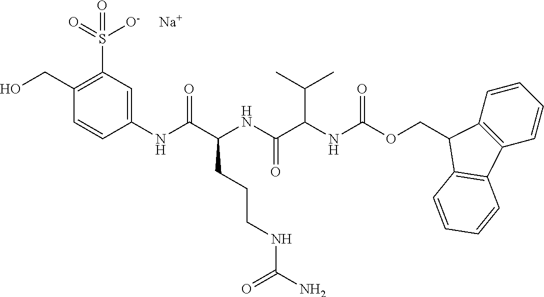 [SMILES: CC(C)C(NC(=O)OCC1c2ccccc2-c2ccccc21)C(=O)N[C@@H](CCCNC(N)=O)C(=O)Nc1ccc(CO)c(S(=O)(=O)[O-])c1 ; [Na+]]

Step 1: sodium 2-(hydroxymethyl)-5-nitro-benzenesulfonate

To a solution of sodium 5-nitro-2-[(E)-2-(4-nitro-2-sulfo-phenyl)vinyl]benzenesulfonate (25.0 g; 52.7 mmol; 1 eq.) in water (336 mL), was introduced a stream of ozone for 1.5 hours. After completion of the reaction, the mixture was purged with argon for 30 minutes in order to remove the excess of ozone. Then, sodium carbonate (39.1 g; 368 mmol) and sodium borohydride (3.99 g; 105 mmol) were added and the orange solution was stirred at r.t. for 16 hours. The reaction mixture was concentrated to dryness to furnish the title compound (39.9 g; 156 mmol) as a brown solid. ¹H NMR (DMSO): δ 4.99 (d, 2H, J=3.6 Hz), 5.36 (t, 1H, J=5.6 Hz), 7.83 (d, 1H, J=8.4 Hz, 8.21 (d, 1H, J=8.4 Hz), 8.45 (s, 1H).

Step 2: sodium 5-amino-2-(hydroxymethyl)benzenesulfonate

Sodium 2-(hydroxymethyl)-5-nitro-benzenesulfonate (26.9 g; 105 mmol) was solubilized in water (493 mL). Then, the reaction mixture was flushed with argon. Pd/C 10% (2.65 g) was added then the black suspension was flushed with argon and then with hydrogen. The reaction mixture was stirred at r.t. for 3.5 days under hydrogen atmosphere. After filtration over Celite® and washing with water and methanol, the filtrate was concentrated to dryness and co-evaporated three times with toluene in order to remove the remaining traces of water. Purification by column chromatography on silica gel using ethyl acetate-methanol (90/10 to 70/30) as eluent afforded the title compound (14.29 g; 63.46 mmol) as a pale yellow solid. ¹H NMR (DMSO): δ 4.52 (d, 2H, J=5.2 Hz), 4.95 (t, 1H, J=5.2 Hz), 5.04 (s, 2H), 6.42 (d, 1H, J=7.6 Hz), 6.93 (d, 1H, J=7.6 Hz), 7.03 (s, 1H).

Step 3: sodium 5-[[(2S)-2-(9H-fluoren-9-ylmethoxycarbonylamino)-5-ureido-pentanoyl]amino]-2-(hydroxymethyl)benzenesulfonate

To a solution of Fmoc-L-Cit-OH (CAS No. 133174-15-9; 882 mg; 2.22 mmol) in DMF (32.5 mL), were added sodium 5-amino-2-(hydroxymethyl)benzenesulfonate (500 mg; 2.22 mmol), HBTU (1.01 g; 2.66 mmol) and DIPEA (917 mL; 5.55 mmol). The reaction mixture was stirred at r.t. for 16 hours, then was concentrated to dryness and co-evaporated with water (2×100 mL). The crude was purified by column chromatography on C₁₈ using neutral method, to afford the title compound (1.0 g; 1.40 mmol) as pale red oil. ¹H NMR (DMSO): δ 4.30-4.12 (m, 4H), 4.74 (d, 2H, J=4.4 Hz), 5.05 (t, 1H, J=5.6 Hz), 5.37 (s, 2H), 5.97 (t, 1H, J=4.8 Hz), 7.34-7.42 (m, 4H), 7.62-7.90 (m, 7H), 8.15 (s, 1H), 10.05 (s, 1H).

Step 4: sodium 5-[[(2S)-2-amino-5-ureido-pentanoyl]amino]-2-(hydroxymethyl)benzenesulfonate

To a solution of sodium 5-[[(2S)-2-(9H-fluoren-9-ylmethoxycarbonylamino)-5-ureido-pentanoyl]amino]-2-(hydroxymethyl)benzenesulfonate (11.2 g; 15.73 mmol) in DMF (224 mL), was added piperidine (3.1 mL; 31.47 mmol). The reaction mixture was stirred at r.t. for 3 hours then water (400 mL) was added. The aqueous layer was extracted with ethyl acetate (2×300 mL) and with DCM (300 mL). Sodium carbonate (5.01 g; 47.1 mmol; 3 eq.) was added to the aqueous layer and the mixture was stirred for 3 hours at r.t. The mixture was lyophilized to give the title compound (6.01 g; 15.73 mmol) as a white solid. ¹H NMR (DMSO): δ 1.55-1.64 (m, 4H), 2.99-3.01 (m, 2H), 3.58 (m, 1H), 4.75 (s, 2H), 5.06 (s, 1H), 5.38 (s, 2H), 5.98 (t, 1H, J=5.6 Hz), 7.38 (d, 1H, J=8.4 Hz), 7.72 (dd, 1H, J=8.4 and 2.4 Hz), 7.86 (d, 1H, J=2.4 Hz), 10.17 (s, 1H).

Step 5: Example 1

To a solution of sodium 5-[[(2S)-2-amino-5-ureido-pentanoyl]amino]-2-(hydroxymethyl)benzenesulfonate (6.01 g; 15.73 mmol) n DMF (150 mL), was added Fmoc-L-Val-OSu (CAS No. 130878-68-1; 6.85 g; 15.69 mmol). The beige solution was stirred at r.t. for 3 hours then the reaction mixture was diluted with saturated sodium hydrogenocarbonate (100 mL) and water (100 mL) and concentrated to dryness. The residue was purified on silica gel using ethyl acetate/methanol 90/10 to 50/50 as eluent to afford the title compound (4.44 g; 6.31 mmol) as a white solid. ¹H NMR (DMSO): 0.85-0.90 (m, 6H), 1.31-1.76 (m, 4H), 1.95-2.06 (m, 1H), 2.91-3.05 (m, 2H), 3.95 (t, 1H, J=8.4 Hz), 4.24-4.35 (m, 3H), 4.37-4.45 (m, 1H), 4.76 (d, 2H, J=6 Hz), 5.07 (t, 1H, J=6.4 Hz), 5.40 (s, 2H), 6.03 (t, 1H, J=5.6 Hz), 7.32-7.46 (m, 6H), 7.67 (d, 1H, J=8 Hz), 7.76 (t, 2H, J=7.2 Hz), 7.88-7.91 (m, 3H), 8.12 (d, 1H, J=7.6 Hz), 10.080 (s, 1H). ¹³C NMR (DMSO): 18.25, 19.24, 26.70, 29.56, 30.45, 39.50, 46.67, 53.17, 60.01, 60.96, 65.66, 117.85, 119.15, 120.05, 125.36, 127.06, 127.62, 128.09, 184.39, 136.79, 140.67, 143.89, 145.34, 156.08, 158.82, 170.37, 171.16, LCMS (2-100 acetonitrile/H₂O+0.1% formic acid): 93.85% Tr=8.4 min. Positiv mode: 682.15 detected (MH⁺). Negativ mode: 680.17 detected (MH⁺)

### EXAMPLE 2

### 5-[[(2S)-2-[[(2S)-2-(9H-fluoren-9-ylmethoxycarbonylamino)-3-methyl-butanoyl]amino]propanoyl]amino]-2-(hydroxymethyl)benzenesulfonic Acid

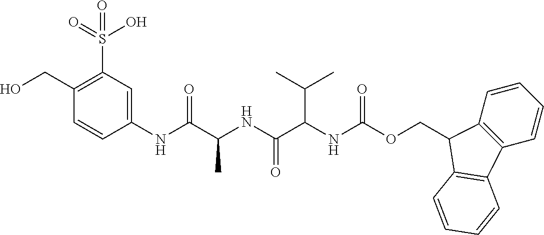 [SMILES: CC(C)C(NC(=O)OCC1c2ccccc2-c2ccccc21)C(=O)N[C@@H](C)C(=O)Nc1ccc(CO)c(S(=O)(=O)O)c1]

Step 1: sodium 5-[[(2S)-2-(tert-butoxycarbonylamino)propanoyl]amino]-2-(hydroxymethyl)benzenesulfonate

To a solution of Boc-L-Ala-OH (CAS No. 15761-38-3; 588 mg; 3.11 mmol) in DMF (38.6 mL), were successively added HATU (1.77 g; 4.67 mmol), sodium 5-amino-2-(hydroxymethyl)benzenesulfonate (771 mg; 3.42 mmol) and DIPEA (1.29 ml; 7.78 mmol). The reaction mixture was stirred for 16 hours at r.t. then concentrated to dryness and co-evaporated with water to afford the crude reaction mixture. The resulting residue was purified by column chromatography on silica gel using ethyl acetate-methanol 75:5 to 80:20 as eluent to afford the title compound (1.17 g; 2.95 mmol) as a white solid. ¹H NMR (DMSO): δ 1.24 (s, 9H), 1.38 (m, 3H), 4.05-1.44 (m, 1H), 4.73 (d, 2H, J=4.8 Hz), 5.04 (t, 1H, J=5.6 Hz), 6.97-7.03 (m, 1H), 7.33 (d, 1H, J=8. Hz), 7.65-7.70 (m, 1H), 7.83 (s, 1H), 9.91 (s, 1H).

Step 2: 5-[[(2S)-2-aminopropanoyl]amino]-2-(hydroxymethyl)benzenesulfonic acid, hydrochloride

Sodium 5-[[(2S)-2-(tert-butoxycarbonylamino)propanoyl]amino]-2-(hydroxymethyl) benzenesulfonate (1.17 g; 2.95 mmol; 1 eq.) was suspended in a solution of HCl 4N in dioxane (10 mL). The mixture wax stirred at r.t. for 2 hours then concentrated to dryness to afford the crude mixture (982 mg; 2.95 mmol) as a white solid. ¹H NMR (DMSO): δ 1.45 (d, 3H, J=5.6 Hz), 3.91-4.0 (m, 1H), 4.76 (s, 2H), 7.41 (d, 1H, J=7.6 Hz), 7.66 (d, 1H, J=7.6 Hz), 7.85 (s, 1H), 8.17 (s, 2H), 10.44 (s, 1H)

Step 3: Example 2

To a solution of 5-[[(2S)-2-aminopropanoyl]amino]-2-(hydroxymethyl)benzenesulfonic acid, hydrochloride (981 mg; 2.95 mmol) in DMF (34.5 ml), were added Fmoc-L-Val-OSu (CAS No. 130878-68-1; 1.29 g; 2.95 mmol; 1 eq.) and DIPEA (975 μL; 5.9 mmol). The mixture was stirred overnight at r.t. then concentrated to dryness and co-evaporated with water to afford the crude mixture. The resulting residue was purified by column chromatography on silica gel using ethyl acetate/methanol 95:5 to 80:20 as eluent to afford Example 2 (1.28 g; 2.072 mmol) as colorless oil ¹H NMR (DMSO); δ 0.80-0.92 (m, 6H), 1.30 (d, 3H, J=6.4 Hz), 2.02-2.10 (m, 1H), 4.17-4.31 (m, 3H), 4.37-4.44 (m, 1H), 4.73 (d, 2H, J=5.6 Hz), 5.04 (t, 1H, J=6.4 Hz), 7.28-7.36 (m, 3H), 7.37-7.47 (m, 3H), 7.66 (d, 1H, J=8.4 Hz), 7.71-7.77 (m, 2H), 7.83-7.85 (m, 1H), 7.88 (d, 2H, J=7.6 Hz), 8.14 (d, 1H, J=6.4 Hz), 9.99 (s, 1H).

### EXAMPLE 3

### Sodium [5-[[(2S)-2-[[(2S)-2-(9H-fluoren-9-ylmethoxycarbonylamino)-3-methyl-butanoyl]amino]-5-ureido-pentanoyl]amino]-2-(hydroxymethyl)phenyl]methanesulfonate

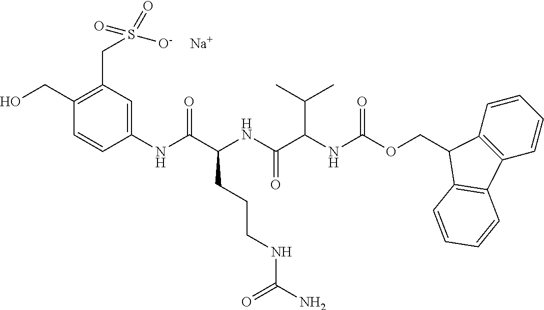 [SMILES: CC(C)C(NC(=O)OCC1c2ccccc2-c2ccccc21)C(=O)N[C@@H](CCCNC(N)=O)C(=O)Nc1ccc(CO)c(CS(=O)(=O)[O-])c1 ; [Na+]]

Step 1: (2S)-2-[[2S)-2-(9H-fluoren-9-ylmethoxycarbonylamino)-3-methyl-butanoyl]amino]-5-ureido-pentanoic acid

Fmoc-L-Val-OSu (CAS No. 130878-68-1; 1.59 g; 3.64 mmol; 1.0 eq.) was added at r.t. to a solution of L(+)-citrulline (0.67 g; 3.82 mmol; 1.05 eq.) and sodium bicarbonate (0.32 g; 3.82 mmol; 1.05 eq.) in a mixture of 1,2-dimethoxyethane (9.5 mL), water (9.5 mL) and THF (4.7 mL). The reaction mixture was stirred at r.t. for 16 hours, then the solvents were removed under vacuum and the residue was acidified with a solution of hydrochloric acid 1N until pH 1. The white suspension was filtered, washed with water (3×40 mL), diethyl ether (3×40 mL) and co-evaporated with acetonitrile (2×150 mL). The resulting white solid was purified by flash chromatography over silica gel using (DCM/acetic acid (99/1))/methanol (10/0 to 7/3) as eluent, then triturated in diethyl ether (2×30 mL). The resulting solid was dissolved in a mixture of methanol (50 ml) and water (50 mL), concentrated to dryness, co-evaporated with acetonitrile (100 mL) and dried to afford the title compound (0.75 g; 1.5 mmol) as a white solid. ¹H NMR (DMSO): δ 0.84-0.88 (m,6H), 1.34-1.41 (m, 2H), 1.51-1.73 (m, 2H), 1.95-2.04 (m, 1H), 2.91-2.96 (m, 2H), 3.88-3.92 (m, 1H), 4.06-4.11 (m, 1H), 4.19-4.31 (m, 3H), 5.36 (s, 2H), 5.91-5.94 (m, 1H), 7.30-7.34 (m, 2H), 7.39-7.45 (m, 3H), 7.75 (t, 2H, J=7.3 Hz), 7.89 (d, 2H, J=7.4 Hz), 8.02 (brs, 1H), 12.56 (brs, 1H)

Step 2: Sodium (2-methoxycarbonyl-5-nitro-phenyl)methanesulfonate

To a solution of sodium sulfite (14.39 g; 114.17 mmol) in water (228 mL), was added a suspension of methyl 2-(bromomethyl)-4-nitrobenzoate (10.43 g; 38.06 mmol) in methanol (37 mL). The reaction mixture was stirred at r.t. for 20 hours and then concentrated to dryness. The resulting crude was purified by flash chromatography over silica gel using ethyl acetate/methanol (10/0 to 6/4) as eluent to afford the title compound (13.9 g; 31.07 mmol) as white solid. ¹H NMR (DMSO); δ 3.82 (s, 3H), 4.29 (s, 2H), 7.87 (d, 1H, J=8.6 Hz), 8.16 (dd, 1H, J=2.5 and 8.6 Hz), 8.25 (d, 1H, J=2.5 Hz)

Step 3: Sodium [2-(hydroxymethyl)-5-nitro-phenyl]methanesulfonate

To a suspension of sodium (2-methoxycarbonyl-5-nitro-phenyl)methanesulfonate (10.1 g; 22.09 mmol) in THF (368 mL), was added lithium borohydride (0.59 g; 24.3 mmol). The reaction mixture was stirred at r.t. for 16 hours, then lithium borohydride (0.27 g; 11.04 mmol) was again added and the reaction mixture was stirred at r.t. for 24 hours. The reaction mixture was diluted with methanol (50 mL) and concentrated to dryness. The crude was purified by flash chromatography over silica gel using ethyl acetate methanol (10/0 to 5/5) as eluent to afford the title compound (5.09 g; 6.52 mmol) as yellow solid. ¹H NMR (DMSO): δ 3.90 (s, 2H), 4.76 (d, 2H, J=5.7 Hz), 5.38 (t, 1H, J=5.6 Hz), 7.66 (d, 1H, J=8.8 Hz), 8.07-8.09 (m, 2H)

Step 4: Sodium [5-amino-2-(hydroxymethyl)phenyl]methanesulfonate

To a solution of sodium [2(hydroxymethyl)-5-nitro-phenyl]methanesulfonate (2 g; 3.57 mmol) in methanol (102 mL) was added Pd/C 10% (0.56 g; 0.55 mmol) under argon. The reaction mixture was purged with hydrogen and stirred at r.t. under atmospheric pressure of hydrogen for 2 hours. The reaction mixture was then filtered over Celite®, washed with methanol (3×50 mL) and concentrated to dryness. The resulting crude was purified by flash chromatography on C₁₈ using acetonitrile/water (2/98 to 50/50) to afford the title compound (1.98 g; 3.46 mmol) as a grey solid. ¹H NMR (DMSO): δ 3.72 (s, 2H), 4.3 (d, 2H, J=5.9 Hz), 4.89 (s, 2H), 4.96 (t, 1H, J=5.9 Hz), 6.38 (dd, 1H, J=2.4 and 8.1 Hz), 6.49 (d, 1H, J =2.4 Hz), 6.9 (d, 1H, J=8.1 Hz)

Step 5: Example 3

To a solution of (2S)-2-[[(2S)-2-(9H-fluoren-9-ylmethoxycarbonylamino)-3-methyl-butanoyl]amino]-5-ureido-pentanoic acid (543 mg; 1.09 mmol) in dry DMF (6 mL), were added HATU (600 mg; 1.58 mmol), sodium hydrogenocarbonate (102 mg; 1.22 mmol) and a suspension of sodium [5-amino-2-(hydroxymethyl)phenyl]methanesulfonate (650 mg; 1.41 mmol) in dry DMF (14 mL). The reaction mixture was stirred at r.t. for 3 hours and then co-evaporated with dioxane (200 mL). The crude was purified by flash chromatography over silica gel using DCM/methanol (10/0 to 5/5) as eluent, followed by flash chromatography on C₁₈ using acetonitrile/water+0.1% formic acid (2/98 to 50/50) as eluent to afford after freeze-drying Example 3 (233 mg; 0.32 mmol) as a white solid. ¹H NMR (DMSO): δ 0.84-0.89 (m, 6H), 1.32-1.49 (m, 2H), 1.4-1.73 (m, 2H), 1.94-2.04 (m, 1H), 2.89-3.09 (m, 2H), 3.81 (s, 2H), 3.91-3.95 (m, 1H), 4.20-4.33 (m, 3H), 4.41-4.46 (m, 1H), 4.47 (s, 2H), 5.07 (brs, 1H), 5.40 (brs, 2H), 5.98 (t, 1H, J=5.7 Hz), 7.21 (d, 1H, J=8.2 Hz), 7.31-7.36 (m, 3H), 7.39-7.45 (m, 3H), 7.63 (dd, 1H, J=2.2 and 8.3 Hz), 7.75 (t, 2H, J=7.5 Hz), 7.89 (d, 2H, J=7.4 Hz), 8.08 (d, 1H, J=7.6 Hz), 10.01-10.8 (m, 1H), LCMS (2-100 ACN/H₂O+0.1% TFA): 93.75% Tr=8.1 min. Negative mode 694.14 defected (M−H)

### EXAMPLE 4

### 2-(chloromethyl)-5-[[(2S)-2-[[(2S)-2-(9H-fluoren-9-ylmethoxycarbonylamino)-3-methyl-butanoyl]amino]-5-ureido-pentanoyl]amino]benzenesulfonic acid

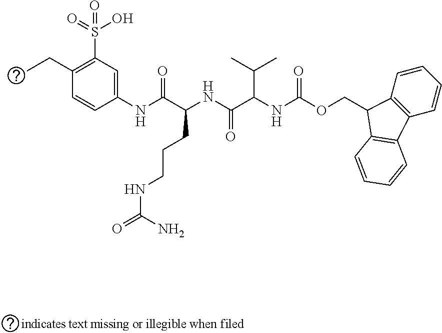 [SMILES: CCc1ccc(NC(=O)[C@H](CCCNC(N)=O)NC(=O)C(NC(=O)OCC2c3ccccc3-c3ccccc32)C(C)C)cc1S(=O)(=O)O]

5-[[(2S)-2-[[(2S)-2-(9H-fluoren-9-ylmethoxycarbonylamino)-3-methyl-butanoyl]amino]-5-ureido-pentanoyl]amino]-2-(hydroxymethyl)benzenesulfonic acid (free acid of Example 1; 300 mg, 0.4263 mmol) was dissolved in anhydrous NMP (6 mL) at r.t. In parallel, a solution of SOCl₂ (206 μL) in NMP (6 mL) was prepared. To the reaction, were added 6 times over a 75 minutes period, a solution 900 μL of the said SoCl₂ solution. After the last addition, the reaction mixture was stirred at r.t. for 15 minutes. The crude product was purified by direct deposit of the reaction mixture on an Oasis column in using the TFA method to afford the title compound (13 mg; 0.1971 mmol) as a white powder. ¹H NMR (400 MHz, dmso-d6) δ ppm 10.15+8.1+7.42+6.0 (s+2d+m, 4H), 7.9 (m, 3H), 7.75 (m, 3H), 7.2+7.31 (2m, 5H), 5.223 (s, 2H), 4.4 (m, 1H), 4.3-4.2 (m, 3H), 3.95 (dd, 1H), 3.0 (m, 2H), 2.0 (m, 1H), 1.7 +1.6 (2m, 2H), 1.48'1.37 (2m, 2H), 0.88 (2d, 6H). HR-ESI+: m/z [M+H]+=700.2199/700.2202 [measured/theoretical]

### EXAMPLE 5

### 2-(chloromethyl)-5-[[(2S0-2-[[(2S)-2-(9H-fluoren-9-ylmethoxycarbonylamino)-3-methyl-butanoyl]amino]propanoyl]amino]benzenesulfonic acid

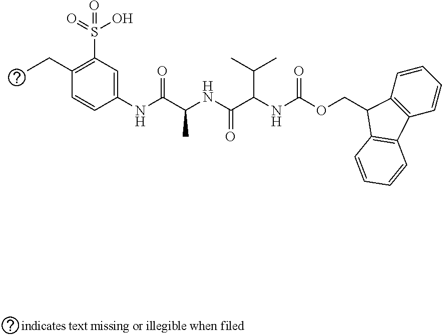 [SMILES: CCc1ccc(NC(=O)[C@H](C)NC(=O)C(NC(=O)OCC2c3ccccc3-c3ccccc32)C(C)C)cc1S(=O)(=O)O]

To a solution of 5-[[(2S)-2-[[(2S)-2-(9H-fluoren-9-ylmethoxycarbonylamino)-3-methyl-butanoyl]amino]propanoyl]amino]-2-(hydroxymethyl)benzenesulfonate (sulfonate obtained from Example 2: 504.1 mg, 0.816 mmol) in NMP (5 mL), were added 6 times over a 75 minutes period, a solution of SOCl₂ (60 μL 0.816 mmol) in NMP (500 μL). The reaction mixture was stirred at r.t. for 15 minutes. The crude product was purified by direct deposit of the reaction mixture on an Oasis column in using the TFA method to afford (337 mg) as a white powder. IR Wavelength (cm⁻1): 3600 to 2400, 1688+1648, 1599, 15118, 1022. UPLC-MS: MS(ESI) m/z [M+H]+=614.17+616.18 (Cl)

### EXAMPLE 6

Sodium 5-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate

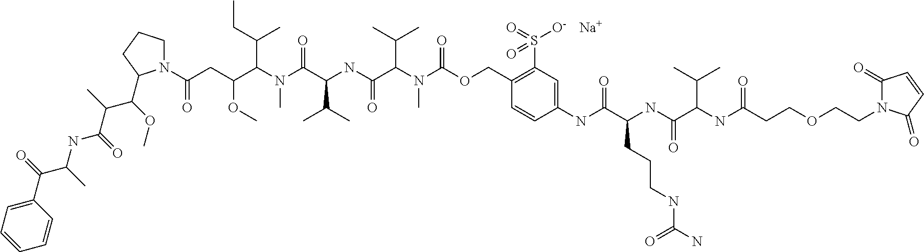 [SMILES: CCC(C)C(C(CC(=O)N1CCCC1C(OC)C(C)C(=O)NC(C)C(=O)c1ccccc1)OC)N(C)C(=O)[C@@H](NC(=O)C(C(C)C)N(C)C(=O)OCc1ccc(NC(=O)[C@H](CCCNC(N)=O)NC(=O)C(NC(=O)CCOCCN2C(=O)C=CC2=O)C(C)C)cc1S(=O)(=O)[O-])C(C)C ; [Na+]]

Step 1: Sodium 5-[[(2S)-2-[[(2S)-2-(9H-fluoren-9-ylmethoxycarbonylamino)-3-methyl-butanoyl]amino]-5-ureido-pentanoyl]amino]-2-[(4-nitrophenoxy)carbonyloxymethyl]benzenesulfonate

To a solution of Example 1 (450 mg; 0.04 mmol) in DMF (6 mL), were added DIEA (1.34 mL; 7.67 mmol) and bis(4-nitrophenyl)carbonate (778 mg; 2.50 mmol). The solution was stirred at r.t. for 2 hours and bis(4-nitrophenyl)carbonate (390 mg; 1.28 mmol) was added. After 1 hour, the solution was concentrated under reduced pressure and the residue was purified by silica gel chromatography (gradient of methanol and acetic acid its DCM) to give the title compound (523 mg; 0.45 mmol). ¹H NMR (400 MHz, dmso-d6) δ ppm 10.2/8.1/5.95 (m, 3H), 8.3 (d, 2H), 7.95 (s, 1H), 7.9 (d, 2H), 7.75 (dd, 1H), 7.75 (m, 2H), 7.65 (d, 2H), 7.4 (d, 4H), 7.35 (d, 1H), 5.7 (s, 2H), 5.35 (brs, 2H), 4.4 (m, 1H), 4.3 (t, 1H), 4.2 (d, 2H), 3.95 (m, 1H), 3 (m, 2H), 2 (m, 1H), 1.8-1.3 (m, 4H), 0.85 (2d, 6H)

Step 2: 5-[[(2S)-2-[[(2S)-2-amino-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxobutyl]-methyl-carbamoyl]-2-methyl-propyl]carbamoyl]-2-methyl-propyl]-methyl-carbamoyl]oxymethyl]benzenesulfonic acid, trifluoroacetic acid salt

To a solution of MMAE (200 mg; 0.28 mmol) in DMF (5.6 mL), was added DIEA (0.19 mL; 1.39 mmol) followed by sodium 5-[[(2S)-2-[[(2S)-2-(9H-fluoren-9-ylmethoxycarbonylamino)-3-methyl-butanoyl]amino]-5-ureido-pentanoyl]amino]-2-[(4-nitrophenoxy)carbonyloxymethyl]benzenesulfonate (472 mg; 0.55 mmol) and HOBt (75 mg; 0.56 mmol). The solution was stirred at r.t. for 3.5 hours, then DEA (0.19 mL; 1.39 mmol) was added. After 2 hours, the solution was concentrated under reduced pressure and the residue was purified by C₁₈ reverse phase preparative-HPLC on Xbridge® by TFA method to give the title compound (99 mg; 0.08 mmol). ¹H NMR (400 MHz, dmso-d6) δ ppm 10.2/10 (2s, 1H), 8.56/8.35 (2m, 1H), 8.1 (d, 1H), 7.9 (brs, 1H), 7.88/7.6 (2d, 1H), 7.68 (brs, 1H), 7.35-7.13 (m, 5H), 7.3 (brs, 1H), 6/5.96 (2t, 1H), 5.5 (m, 2H), 5.4 (brs, 2H), 4.8-4.15 (m, 5H), 4.05-3.85 (m, 2H), 3.8-3.6 (5s, 9H), 3.62-3.5 (m, 2H), 3.1-2.8 (m, 5H), 3 (brs, 3H), 2.4/2.3 (d+dd, 2H), 2.2-1.9 (m, 3H), 1.9-1.75 (m, 11H), 1.05/1 (2d, 9H), 1-0.75 (m, 21H)

Step 3: Example 6

To a solution of 5-[[(2S)-2-[[(2S)-2-amino-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid, TFA salt (22 mg; 0.018 mmol) in DMF (0.18 mL), were added DIEA (6.3 μL mL; 36 μmmol) and 1-[2-[3-(2,5-dioxopyrrolidin-1-yl)-3-oxo-propoxy]ethyl]pyrrole-2,5-dione (6.2 mg; 20 μmol). The solution was stirred at r.t. for 4.5 hours. The reaction was purified by C₁₈ reverse phase preparative-HPLC on Xbridge® column using NH₄HCO₃ method to give Example 6 (9.6 mg; 6.8 μmol). HRMS (ESI) [M+H]⁺ found=1420.7041 (δ=−0.3 ppm)

### EXAMPLE 7

### Sodium 5-[[(2S)-2-[[(2S)-2-[6-(2,5-dioxopyrrol-1-yl)hexanoylamino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate

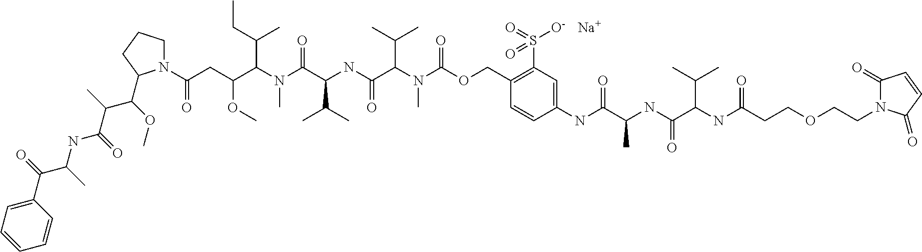 [SMILES: CCC(C)C(C(CC(=O)N1CCCC1C(OC)C(C)C(=O)NC(C)C(=O)c1ccccc1)OC)N(C)C(=O)[C@@H](NC(=O)C(C(C)C)N(C)C(=O)OCc1ccc(NC(=O)[C@H](C)NC(=O)C(NC(=O)CCOCCN2C(=O)C=CC2=O)C(C)C)cc1S(=O)(=O)[O-])C(C)C ; [Na+]]

To a solution of 5-[[(2S)-2-[[(2S)-2-amino-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid, TFA salt (obtained from Step 2 of Example 6: 30 mg; 0.025 mmol) in DMF (0.25 m L) was added DIEA (8.7 μL mL; 36 μmmol) and (2,5-dioxopyrrolidin-1-yl) 6-(2,5-dioxopyrrol-1-yl)hexanoate (8.46 mg, 0.027 mmol). The solution was stirred at r.t. for 3.5 hours. The reaction was purified by C₁₈ reverse phase preparative-HPLC on Xbridge® column using NH₄HCO₃ method to give Example 7 (20 mg; 14.09 μmol). HRMS (ESI) [M+H]⁺ found=1418.7253 (δ=0.1 ppm)

### EXAMPLE 8

### sodium 5-[[(2S)-2-[[(2S)-2-[[2-[2-[2-(2azidoethoxy)ethoxy]ethoxy]acetyl]amino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate

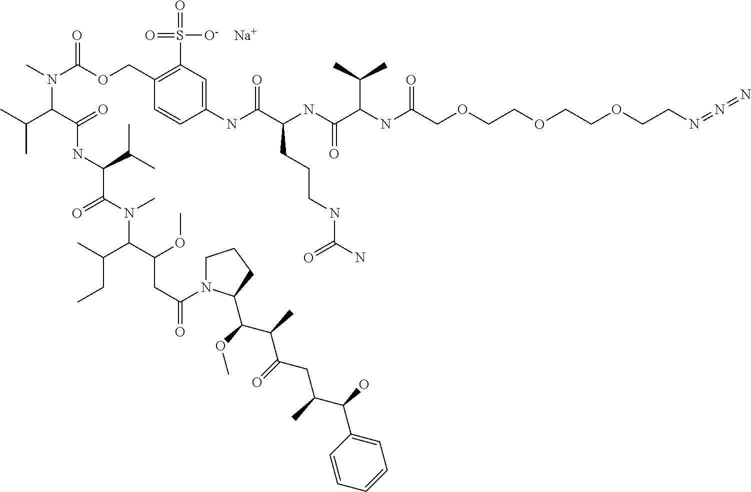

To a solution of 2-[2-[2-(2-azidoethoxy)ethoxy]ethoxy]acetic acid (64 mg; 0.027 mmol) in DMF (125 μL), were added DIEA (21.7 μL; 124 μmmol) and TSTU (7.88 mg; 0.026 mmol). After completion of the activation, a solution of 5-[[(2S)-2-[[(2S)-2-amino-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid, TFA salt (30 mg; 0.025 mmol) in DMF (0.15 mL) was added. The solution was stirred at r.t. for 2 hours. The reaction was purified by C₁₈ reverse phase preparative-HPLC on Xbridge® column using NH₄HCO₃ method to give Example 8 (1 mg; 11.10 μmol). HRMS (ESI) [M+H]⁺ found=1440.7382 (δ=−2.6 ppm)

### EXAMPLE 9

### 5-[[(2S)-2-[[1-[2-[2-(2,5-dioxopyrrol-1-yl)ethoxy]ethylcarbamoyl]cyclobutanecarbonyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid

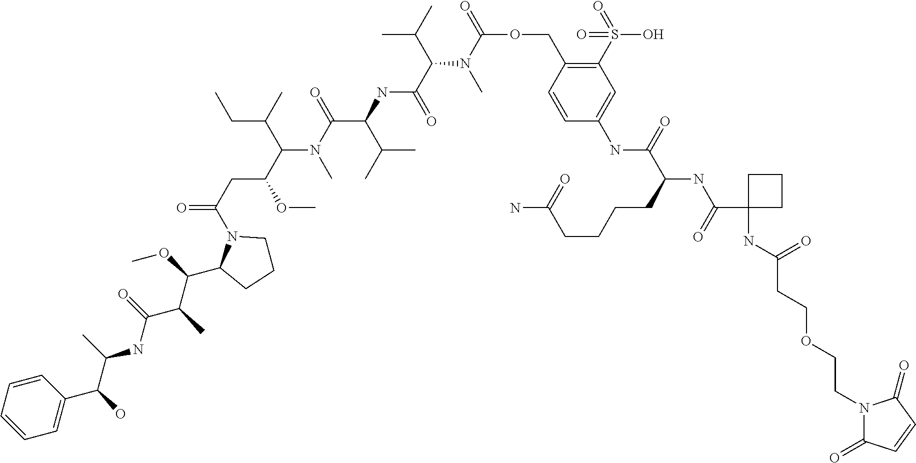 [SMILES: CCC(C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCCC(N)=O)NC(=O)C2(NC(=O)CCOCCN3C(=O)C=CC3=O)CCC2)cc1S(=O)(=O)O)C(C)C]

Step 1: sodium 5-[[(2S)-2-(9H-fluoren-9-ylmethoxycarbonylamino)-5-ureido-pentanoyl]amino]-2-[(4-nitrophenoxy)carbonyloxymethyl]benzenesulfonate

To a solution of sodium 5-[[(2S)-2-(9H-fluoren-9-ylmethoxycarbonylamino)-5-ureido-pentanoyl]amino]-2-(hydroxymethyl)benzenesulfonate (261 mg; 0.448 mmol) in DMF (5.75 mL), were successively added bis(4-nitrophenyl) carbonate (1.09 g; 3.58 mmol) and DIPEA (1.87 mL; 10.75 mmol). The reaction mixture was stirred at r.t. for 16 hours, then was concentrated so dryness to afford the crude mixture. The crude product was purified by silica gel chromatography (gradient of methanol containing 2% AcOH in DCM) to afford the title compound (137 mg: 0.183 mmol) as a white solid. UPLC-MS: MS(ESI): m/z [M+H]+=748.48

Step 2: sodium 5-[[(2S)-2-(9H-fluoren-9-ylmethoxycarbonylamino)-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate

To a solution of 5-[[(2S)-2-(9H-fluoren-9-ylmethoxycarbonylamino)-5-ureido-pentanoyl]amino]-2-[(4-nitrophenoxy)carbonyloxymethyl]benzenesulfonic acid (64.5 mg; 0.0863 mmol) in DMF (1.42 mL), were successively added MMAE (61.9 mg; 0.0863 mmol), DIPEA (75.1 μL; 0.431 mmol) and HOBt (23.3 mg; 0.173 mmol). The reaction mixture was stirred at r.t. for 16 hours. The crude product was purified by C₁₈ reverse phase prep-HPLC by direct deposit of the reaction mixture on the Xbridge® column and using the TFA method to afford the title compound (33.5 mg; 0.0253 mmol). UPLC-MS: MS(ESI): m/z [M+H]+=1326.88, [M+Na]+=1348.09

Step 3: 5-[[(2S)-2-amino-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid, TFA salt

To a solution of sodium 5-[[(2S)-2-(9H-fluoren-9-ylmethoxycarbonylamino)-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate (36.8 mg; 0.0292 mmol) in DMF (750 μL), was added piperidine (23.1 μL; 0.234 mmol). The reaction mixture was stirred at r.t. for 17 hours. The crude product solution was purified by C₁₈ reverse phase prep-HPLC by direct deposit of the reaction mixture on the Xbridge® column and using the TFA method to afford the title compound (17 mg; 0.0139 mmol). UPLC-MS: MS(ESI): m/z [M+H]+=1038.10, [M+Na]+=1059.85

Step 4: Example 9

To a solution of 5-[[(2S)-2-amino-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid, TFA salt (24.5 mg; 0.0215 mmol) in DMF (735 μL), were successively added Preparation 1 (10.5 mg; 0.0258 mmol) and DIPEA (19 μL; 0.108 mmol). The reaction mixture was stirred at r.t. for 16 hours. The crude product solution was purified by C₁₈ reverse phase prep-HPLC by direct deposit of the reaction mixture on the Xbridge® column and using the TFA method to afford Example 9 (19.3 mg; 0.0147 mmol) as a white powder. HR-ESI+: m/z [M+H]+=1396.7040/1396.7074 [measured/theoretical]

### EXAMPLE 10

### 5-[[(2S)-2-[[(2S)-2-[3-[2-(2,5-dioxopyrrol-1-yl)ethoxy]propanoylamino]-3-methyl-butanoyl]amino]propanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid

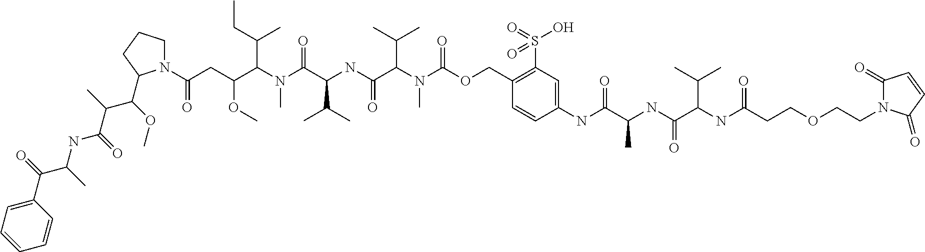 [SMILES: CCC(C)C(C(CC(=O)N1CCCC1C(OC)C(C)C(=O)NC(C)C(=O)c1ccccc1)OC)N(C)C(=O)[C@@H](NC(=O)C(C(C)C)N(C)C(=O)OCc1ccc(NC(=O)[C@H](C)NC(=O)C(NC(=O)CCOCCN2C(=O)C=CC2=O)C(C)C)cc1S(=O)(=O)O)C(C)C]

Step 1: 5[[(2S)-2-[[(2S)-2-(9H-fluoren-9-ylmethoxycarbonylamino)-3-methyl-butanoyl]amino]propanoyl]amino]-2-[(4-nitrophenoxy)carbonyloxymethyl]benzenesulfonic acid

To a suspension of Example 2 (1.28 g; 2.07 mmol) in THF (65 mL), was added pyridine (875 μL; 10.8 mmol) followed by 4-nitrophenyl chloroformate (1.09 g; 5.41 mmol). The mixture was stirred overnight at r.t. Then additional 4-nitrophenyl chloroformate (1.09 g; 5.41 mmol; 2.5 eq.) was added. After 5 hours stirring at r.t., the mixture was concentrated to dryness then purified by column chromatography on C₁₈ using water/acetonitrile 90/10 to 0/100 as eluent in 30 minutes. Acetonitrile of the combined tubes was removed, and the rest was lyophilized to afford the title compound (650 mg; 0.83 mmol) as a white solid. ¹H NMR (DMSO): δ 0.88 (m, 6H), 1.31 (d, 3H, J=4.8 Hz), 1.97-2.03 (m, 1H), 3.92 (t, 1H, J=6.8 Hz), 4.23 (s, 2H), 4.24-4.34 (m, 1H), 4.42 (t, 1H, J=5.6 Hz), 5.69 (s, 2H), 7.30-7.47 (m, 6H), 7.62 (d, 2H, J=8 Hz), 7.72-7.76 (m, 3H), 7.89 (d, 2H, J=6.4 Hz), 7.94 (s, 1H), 8.18 (d, 1H, J=5.6 Hz), 8.33 (d, 2H, J=7.6 Hz), 10.11 (s, 1H). ¹³C NMR (DMSO): δ 18.01, 18.26, 19.21, 30.4, 46.66, 49.05, 59.91, 65.67, 67.82, 117.7, 119.1, 120.06, 122.66, 125.37, 126.33, 127.05, 127.62, 128.0, 138.06, 140.67, 143.77, 143.86, 145.1, 146.23, 151.96, 155.47, 156.12, 171.0, 171.15. LCMS (2-100 ACN/H₂O+0.05% TFA): 90.41% Tr=12.7 min. Positiv mode 578.41 detected. Negativ mode 759.17 detected

Step 2: 5-[[(2S)-2-[[(2S)-2-(9H-fluoren-9-ylmethoxycarbonylamino)-3-methyl-butanoyl]amino]propanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S0-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid

To a solution of 5-[[(2S)-2-[[(2S)-2-(9H-fluoren-9-ylmethoxycarbonylamino)-3-methyl-butanoyl]amino]propanoyl]amino]-2-[(4-nitrophenoxy)carbonyloxymethyl]benzenesulfonic acid (29.3 mg; 0.0374 mmol) in DMF (1.20 mL), were successively added MMAE (26.9 mg; 0.0374 mmol), DIPEA (32.6 μL; 0.187 mmol) and HOBt (10.1 mg; 0.075 mmol). The reaction mixture was stirred at r.t. for 16 hours. The crude product was purified by C₁₈ reverse phase prep-MPLC by direct deposit of the reaction mixture on the Xbridge® column and using the TFA method to afford the title compound (35.4 mg; 0.0264 mmol). UPLC-MS: MS(ESI): m/z [M+H]+=1340.9

Step 3: 5-[[(2S)-2-[[(2S)-2-amino-3-methyl-butanoyl]amino]propanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid, TFA salt

To a solution of 5-[[(2S)-2-[[(2S)-2-(9H-fluoren-9-ylmethoxycarbonylamino)-3-methyl-butanoyl]amino]propanoyl]amino]-2-[[[(1S)-1-[[(1S)-1-[[(1S,2R)-4-[(2S)-2-[(1R,2R)-3-[[(1R,2S)-2-hydroxy-1-methyl-2-phenylethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid (50.1 mg; 0.0374 mmol) in DMF (1.5 mL), was added piperidine (29.6 μL; 0.299 mmol). The reaction mixture was stirred at r.t. for 1 hour. The crude product solution was purified by C₁₈ reverse phase prep-MPLC by direct deposit of tire reaction mixture on the Xbridge® column and using the TFA method to afford the title compound (23 mg; 0.0187 mmol) as a white powder. HR-ESI+: m/z [M+H]+=111.76235/1117.6218. [M+2H]/2+=559.3146/559.33148 [measured/theoretical]

Step 4: Example 10

To a solution of 5-[[(2S)-2-[[(2S)-2-amino-3-methyl-butanoyl]amino]propanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4oxo-butyl]-methyl-carbamoyl]-2-methyl-propyl]carbamoyl]-2-methyl-propyl]-methyl-carbamoyl]oxymethyl]benzenesulfonic acid, TFA salt (10.4 mg; 0.00885 mmol) in DMF (0.34 mL), were successively added (2,5-dioxopyrrolidin-1-yl) 3-[2-(2,5-dioxopyrrol-1-yl)ethoxy]propanoate (2.9 mg; 0.00929 mmol) and DIPEA (7.36 μL; 0.0422 mmol). the reaction mixture was stirred at r.t. for 16 hours. The crude product was purified by C₁₈ reverse phase prep-HPLC by direct deposit of the reaction mixture on the Xbridge® column and using the TFA method to afford Example 10 (7.7 mg; 0.0059 mmol) as a white powder. HR-ESI+: m/z [M+H]+=1312.6701/1312.6750, [measured/theoretical]

### EXAMPLE 11

### 5-[[(2S)-2-[[3-[2-[2-(2,5-dioxopyrrol-1-yl)ethoxy]ethylcarbamoyl]oxetane-3-carbonyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid

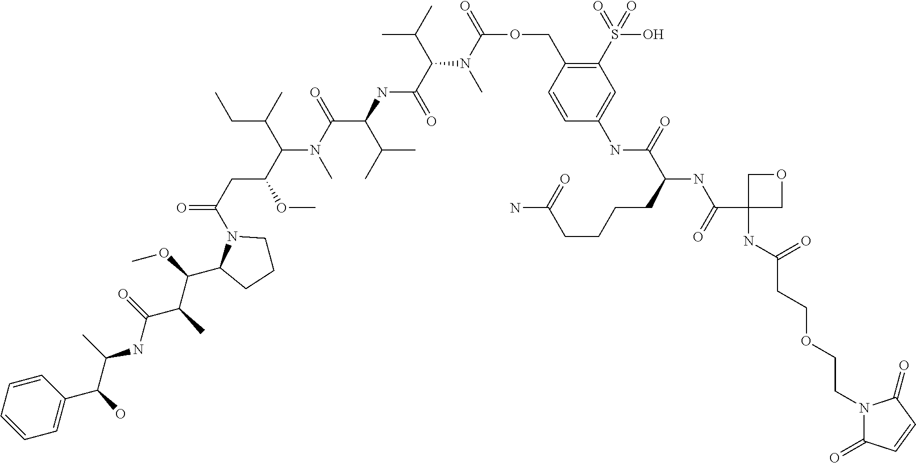 [SMILES: CCC(C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCCC(N)=O)NC(=O)C2(NC(=O)CCOCCN3C(=O)C=CC3=O)COC2)cc1S(=O)(=O)O)C(C)C]

To a solution of 5-[[(2S)-2-amino-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid; 2,2,2-trifluoroacetic acid (16 mg; 0.0131 mmol) in DMF (480 μL), were successively added Preparation 2 (10.24 mg; 0.0250 mmol) and DIPEA (11.4 μL; 0.0657 mmol). The reaction mixture was stirred at r.t. for 16 hours. The crude product solution was purified by C₁₈ reverse phase prep-HFLC by direct deposit of the reaction mixture on the Xbridge® column and using the TFA method to afford Example 11 (4.4 mg; 0.0031 mmol) as a white powder. HR-ESI+: m/z [M+2H]2+=699.8477/699.8472 [measured/theoretical]

### EXAMPLE 12

### 5-[[(2S)-2-[[3-[2-[2-(2,5-dioxopyrrol-1-yl)ethoxy]propanoylamino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid

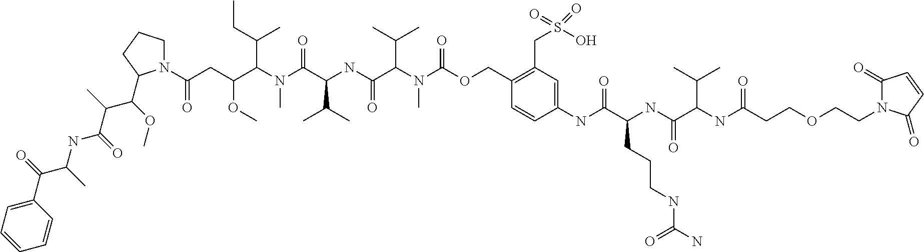 [SMILES: CCC(C)C(C(CC(=O)N1CCCC1C(OC)C(C)C(=O)NC(C)C(=O)c1ccccc1)OC)N(C)C(=O)[C@@H](NC(=O)C(C(C)C)N(C)C(=O)OCc1ccc(NC(=O)[C@H](CCCNC(N)=O)NC(=O)C(NC(=O)CCOCCN2C(=O)C=CC2=O)C(C)C)cc1CS(=O)(=O)O)C(C)C]

Step 1: Sodium -[5-[[(2S)-2-[[(2S)-2-(9H-fluoren-9-ylmethoxycarbonylamino)-3-methyl-butanoyl]amino]-5-ureido-pentanoyl]amino]-2-[(4-nitrophenoxy)carbonyloxymethyl]phenyl]methanesulfonate

To a solution of Example 3 (30 mg; 0.0418 mmol) in DMF (0.4 mL) was added DIEA (87 μL; 70.50) mmol) and bis(4-nitrophenyl)carbonate (51 mg; 0.167 mmol). The solution was stirred at r.t. for 2 hours and bis(4-nitrophenyl)carbonate (25 mg; 0.08 mmol) was again added. After 1 hour at r.t., the solution was concentrated under reduced pressure and the residue was purified by silica gel chromatography (gradient of methanol in DCM) to give the title compound (25 mg; 0.030 mmol).

Step 2: [5-[[(2S)-2-[[(2S)-2-(9H-fluoren-9-ylmethoxycarbonylamino)-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenyl]methanesulfonic acid

To a solution of MMAE (10 mg; 0.0139 mmol) in DMF (0.6 mL), was added DIEA (12 μL; 0.0696 mmol) followed by sodium [5-[[(2S)-2-[[(2S)-2-(9H-fluoren-9-ylmethoxycarbonylamino)-3-methyl-butanoyl]amino]-5-ureido-pentanoyl]amino]-2-[(4-nitrophenoxy)carbonyloxymethyl]phenyl]methanesulfonate (24 mg; 0028 mmol) and HOBt (3.77 mg; 0.0279 mmol). The solution was stirred at r.t. for 3.5 hours and be solution was directly purified by C₁₈ reverse phase preparative-HPLC on Xbridge® by TFA method to give the title compound (5.4 mg; 0.003 mmol). HRMS (ESI) [M+H]+ found=1439.7558 (δ=1.9 ppm)

Step 3: [5-[[(2S)-2-[[(2S)-2-amino-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenyl]methanesulfonic acid, TFA salt

[5-[[(2S)-2-[[(2S)-2-(9H-fluoren-9-ylmethoxycarbonylamino)-3-methyl-butanoyl]amino]-5-ureido-pentanoyl]amino]-2-[[[(1S)-2-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenyl]methanesulfonic acid (5 mg; 3.4 μmol ) was dissolved in DMF (0.2 mL) then DEA (0.7 μL; 6.8 mmol) was added. The reaction was stirred at r.t. for 1 hour and the solution was purified by C₁₈ reverse phase preparative-HPLC on Xbridge® by TFA method to give the title compound (3.5 mg; 2.6 μmol).

Step 4: Example 12

To a solution of [5-[[(2S)-2-[[(2S)-2-amino-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenyl]methanesulfonic acid, TFA salt (3.5 mg; 2.6 μmol) in DMF (15 μL), were added DIEA (2.5 μL; 13 μmmol) and 1-[2-[3-(2,5-dioxopyrrolidin-1-yl)-3-oxo-propoxy]ethyl]pyrrole-2,5-dione (0.9 mg; 2.9 μmol). The solution was stored at r.t. for 2.5 hours. The reaction was purified by C₁₈ reverse phase preparative-HPLC on Xbridge® column using NH₄HCO₃ method to give Example 12 (2.8 mg; 1.8 μmol). HRMS (ESI) [M+H]+ found=1412.7397 (δ=1.1 ppm)

### EXAMPLE 13

### 5-[[(2S)-2-[[(2S)-2-(9H-fluoren-9-ylmethoxycarbonylamino)-3-methyl-butanoyl]amino]-5-ureido-pentanoyl]amino]-2-(iodomethyl)benzenesulfonic acid

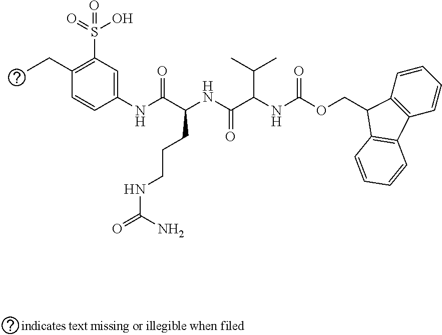 [SMILES: CCc1ccc(NC(=O)[C@H](CCCNC(N)=O)NC(=O)C(NC(=O)OCC2c3ccccc3-c3ccccc32)C(C)C)cc1S(=O)(=O)O]

To a solution of Example 4 (100 mg; 123 μmol) in solution in acetone (6 mL) was added sodium iodide (51 mg; 340 μmol). The reaction mixture was sorted at r.t. for 20 hours. The solvent was evaporated, and the compound was used in the next step without further work-up. UPLC-MS: [M+H]+792.65; [M+Na]+ 814.49.

### EXAMPLE 14

### [4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]-2-sulfo-phenyl]methyl-[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-dimethyl-ammonium; 2,2,2-trifluoroacetate

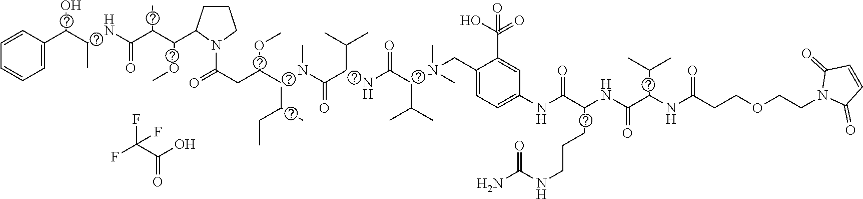

Step 1: [4-[[(2S)-2-[[(2S)-2-(9H-fluoren-9-ylmethoxycarbonylamino)-3-methyl-butanoyl]amino]-5-ureido-pentanoyl]amino]-2-sulfo-phenyl]methyl-[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-dimethyl-ammonium; iodide

To a solution of (2S)-2-[[(2S)-2-(dimethylamino)-3-methyl-butanoyl]amino]-N-[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-N,3-dimethyl-butanamide (auristatin E) (42.1 mg; 57.5 μmol) in DMF (10 mL), were successively added Example 13 (152 mg; 95.8 μmol) and DIPEA (83.4 μL; 365 μmol). The reaction was stirred at r.t. for 17 hours. The desired product was observed by UPLC-MS and the solution was used without work-up in the next step. UPLC-MS [M+H]+ 1396.31; [M+Na]+ 1418.37

Step 2: [4-[[(2S)-2-[[(2S)-2-amino-3-methyl-butanoyl]amino]-5-ureido-pentanoyl]amino]-2-sulfo-phenyl]methyl-[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-dimethyl-ammonium; 2,2,2-trifluoroacetate; 2,2,2-trifluoroacetic acid

To the solution of [4-[[(2S)-2-[[(2S)-2-(9H-fluoren-9-ylmethoxycarbonylamino)-3-methyl-butanoyl]amino]-5-ureido-pentanoyl]amino]-2-sulfo-phenyl]methyl-[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-dimethyl-ammonium; iodide in DMF, was added piperidine (45.4 μL; 460 μmol) and the reaction was stirred at r.t. for 1 hour. The crude product was purified by C18 reverse phase prep-HPLC by direct deposit of the reaction mixture on the Xbridge® column and using the TFA method to afford the desired product (15 mg; 10.7 μmol) as a white solid. UPLC-MS: [M+H]+ 1174.51; [M+Na]+ 1197.03

Step 3: Example 14

To a solution of [4-[[(2S)-2-[[(2S)-2-(9H-fluoren-9-ylmethoxycarbonylamino)-3-methyl-butanoyl]amino]-5-ureido-pentanoyl]amino]-2-sulfo-phenyl]methyl-[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-dimethyl-ammonium; iodide (15 mg; 10.7 μmol) in solution in DMF (450 μL), were successively added (2,5-dioxopyrrolidin-1-yl)-3-[2-(2,5-dioxopyrrol-1-yl)ethoxy]propanoate (3.7 mg; 11.8 μmol) and DIPEA (9.3 μL; 53.5 μmol). The solution was stirred at r.t. for 1 hour. The crude product was purified by C18 reverse phase prep-HPLC by direct deposit of the reaction mixture on the Xbridge® column and using the TFA method to afford Example 14 (15.7 mg; 12.1 μmol) as a white solid. HRMS (ESI) [M—CF₃CO₂]+ 1368.7460 (δ=−1.7 ppm)

### EXAMPLE 23

### N-({[4-({N-[6-(2,5-dioxo-2,5-dihydro-1H-pyrrol-1-yl)hexanoyl]-L-valyl-L-alanyl}amino)-2-sulfophenyl]methoxy}carbonyl)-N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide

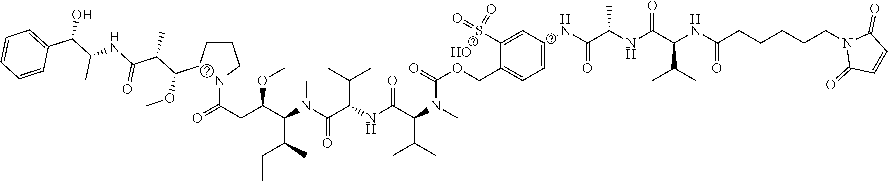 [SMILES: CC[C@H](C)[C@@H]([C@@H](CC(=O)N1CCCC1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCCCCN2C(=O)C=CC2=O)C(C)C)cc1S(=O)(=O)O)C(C)C]

To a solution of 5-[[(2S)-2-[[(2S)-2-amino-3-methyl-butanoyl]amino]propanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid, TFA salt (12.7 mg; 0.0103 mmol; obtained from Step 3 of Example 10) in DMF (0.4 mL), were added DIEA (9 μL; 51 μmol) and (2,5-dioxopyrrolidin-1-yl) 6-(2,5-dioxopyrrol-1-yl)hexanoate (3.5 mg; 11.3 μmol). The solution was stirred at r.t. for 6 hours. The reaction was purified by C₁₈ reverse phase preparative-HPLC on Xbridge® column using TFA method to give Example 23. IR (cm⁻¹): 3278, 1768/1703, 1631, 1159, 829/696. RMN ¹H (500 MHz, DMSO-d6) δ ppm 9.96 (s, 1H), 8.1 (s, 1H), 7.87 (m, 2H), 7.68 (m, 1H), 7.6 (d, 1H), 7.31 (m, 2H), 7.26 (m, 3H), 7.17 (m, 1H), 6.99 (s, 2H), 5.48 (m, 2H), 4.74 (m, 1H), 4.49 (m, 1H), 4.46 (m, 1H), 4.41 (m, 1H), 4.38 (m, 1H), 4.33 (m, 1H), 4.19 (m, 1H), 3.98 (m, 1H), 3.98 (m, 1H), 3.77 (m, 1H), 3.6 (m, 2H), 3.56 (m, 1H), 3.46 (m, 1H), 3.36 (m, 2H), 3.32 (m, 1H), 3.26 (m, 2H), 3.24 (m, 3H), 3.23 (m, 2H), 3.19 (m, 3H), 3.13 (m, 2H) 3.04 (m, 1H), 2.98 (m, 3H), 2.94 (m, 1H), 2.9 (m, 1H), 2.86 (m, 1H), 2.37 (br dd, 2H), 2.12 (m, 2H), 2.12 (m, 2H), 2.06 (m, 2H), 1.97 (m, 2H), 1.49 (m, 9H), 1.29 (d, 3H), 0.89 (m, 27H). HRMS (ESI) [M+H]⁺ found=1310.6893 (δ=−4.5 ppm)

### EXAMPLE 24

### N-[6-(2,5-dioxo-2,5-dihydro-1H-pyrrol-1-yl)hexanoyl]-L-valyl-N⁵-carbamoyl-N-(4-{[({[(4S)-4,11-diethyl-9-hydroxy-3,14-dioxo-3,4,12,14-tetrahydro-1H-pyrano[3′,4′:6,7]indolizino[1,2-b]quinolin-4-yl]oxy}carbonyl)oxy]methyl}-3-sulfophenyl-L-ornithinamide

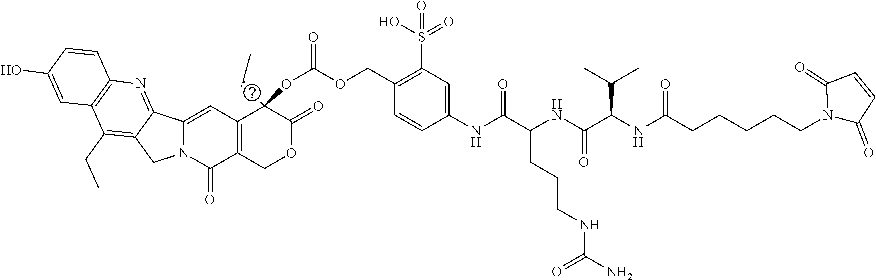 [SMILES: CCc1c2c(nc3ccc(O)cc13)-c1cc3c(c(=O)n1C2)COC(=O)[C@@]3(CC)OC(=O)OCc1ccc(NC(=O)C(CCCNC(N)=O)NC(=O)[C@H](NC(=O)CCCCCN2C(=O)C=CC2=O)C(C)C)cc1S(=O)(=O)O]

Step 1: 5-[[(2S)-2-[[(2S)-2-amino-3-methyl-butanoyl]amino]-5-ureido-pentanoyl]amino]-2-hydroxymethyl)benzenesulfonic acid

To a solution of Example 1 (200 mg; 0.284 mmol) in DMF (8 mL), was added DEA (212 μL; 2.053 mmol) and the reaction was stirred at r.t. for 1 hour. The excess of DEA was evaporated under vacuum and the solution of crude expected compound in DMF was use directly in the next step.

Step 2: 5-[[(2S)-2-[[(2S)-2-[6-(2,5-dioxopyrrol-1-yl)hexanoylamino]-3-methyl-butanoyl]amino]-5-ureido-pentanoyl]amino]-2-(hydroxymethyl)benzenesulfonic acid

To the solution (8 mL) of the compound from the previous step, were added DIEA (102 μL; 0.5866 mmol) and (2,5-dioxopyrrolidin-1-yl) 6-(2,5-dioxopyrrol-1-yl)hexanoate (99.4 mg; 0.3226 mmol). The reaction was stirred at r.t. for 18 hours. The reaction was purified by C₁₈ reverse phase preparative-HPLC on Xbridge® column using TFA method to give after lyophilization the expected compound. IR (cm⁻¹): between 3674 and 2999, 1768 (weak)+1699+1641, 1238/1141, 825 and 694. RMN ¹H (400 MHz, DMSO-d6) δ ppm 9.97 (s, 1H), 8.04 (d, 1H), 7.85 (d, 1H), 7.77 (d, 1H), 7.65 (dd, 1H), 7.32 (d, 1H), 6.99 (s, 2H), 6.02 (m, 1H), 5.18 (m, 1H), 4.72 (s, 2H), 4.36 (m, 1H), 4.21 (dd, 1H), 3.37 (t, 2H), 2.98 (m, 2H), 2.16 (m, 2H), 1.97 (m, 1H), 1.65 (m, 2H), 1.39 (m, 8H), 0.83 (dd, 6H). HRMS (ESI) [M+H]+ found=653.2572 (δ=−4.2 ppm)

Step 3: tert-butyl [(19S)-10,19-diethyl-19-hydroxy-14,18-dioxo-17-oxo-3,13-diazapentacyclo[11.8,0.02,11.04,9.015,20]henicosa-1(21),2,4(9),5,7,10,15(20)-heptaen-7-yl]carbonate

To a suspension of (19S)-10,19-diethyl-7,19-dihydroxy-17-oxo-3,13-diazapentacyclo [11.8,0.02,11.04,9.015,20]henicosa-1(21),2,4(9),5,7,10,15(20)-heptaen-14,18-dione (220 mg; 0.5607 mmol) in DCM (22 mL), were added tert-butoxycarbonyl tert-butyl carbonate (128.5 mg; 0.5887 mmol) as a powder and pyridine (91 μL; 1.121 mmol). The yellow suspension was stirred at r.t. for 18 hours. The solvent and excess of pyridine were evaporated off under vacuum and the crude was purified by chromatography on silica (DCM/MeOH) to give the expected compound as a yellow powder. IR (cm⁻1): 3700-3000, 1753, 1659, 1254/1142. RMN ¹H (400 MHz, DMSO-d6) δ0 ppm 8.21 (d, 1H), 8.09 (d, 1H), 7.74 (dd, 1H), 7.33 (s, 1H), 6.5 (s, 1H), 5.38 (2s, 4H), 3.2 (q, 2H) 1.86 (m, 2H), 1.4 (s, 9H), 1.3 (t, 3H), 0.88 (t, 3H)

Step 4: tert-butyl [(19S)-19-chlorocarbonyloxy-10,19-diethyl-14,18-dioxo-17-oxo-3,13-diazapentacyclo[11.8,0.02,11.04,9.015,20]henicosa-1(21),2,4(9),5,7,10,15(20)-heptaen-7-yl]carbonate

To a solution of tert-butyl [(19S)-10,19-diethyl-19-hydroxy-14,18-dioxo-17-oxo-3,13-diazapentacyclo[11.8,0.02,11.04,9.015,20]henicosa-1(21),2,4(9),5,7,10,15(20)-heptaen-7-yl]carbonate (30 mg; 0.0609 mmol) in DCM (2 mL), were added DMAP (22.3 mg; 0.1827 mmol) followed by triphosgene (7.2 mg; 0.0244 mmol). The reaction was stirred at r.t. for 30 minutes and this solution was used as such in next step.

Step 5: 2-[[(19S)-7-tert-butoxycarbonyloxy-10,10-diethyl-14,18-dioxo-17-oxo-3,13-diazapentacyclo[11.8,0.02, 11.04,9.015,20]henicosa-1(21),2,4(9),5,7,10,15(20)-heptaen-19-yl]oxycarbonyloxymethyl]-5-[[(2S)-2-[[(2S)-2-[-(2,5-dioxopyrrol-1-yl)hexanoylamino]-3-methyl-butanoyl]amino]-5-ureido-pentanoyl]amino]benzenesulfonic acid

To a solution of tert-butyl [(19S)-19-chlorocarbonyloxy-10,19-diethyl-14,18-dioxo-17-oxa-3,13-diazapentacyclo[11.8,0.02,11.04,9.015,20]henicosa-1(21),2,4(9),5,7,10,15(20)-heptaen-7-yl]carbonate in solution in DCM (2 mL), were added DMAP (22.3 mg; 0.1827 mmol) followed by 5-[[(2S)-2-[[(2S)-2-[6-(2,5-dioxopyrrol-1-yl)hexanoylamino]-3-methyl-butanoyl]amino]-5-ureido-pentanoyl]amino]-2-(hydroxymethyl)benzenesulfonic acid (31.80 mg; 0.04873 mmol; obtained according to Step 2 above). The reaction was stirred at r.t. for 14 hours. The reaction was purified by C₁₈ reverse phase preparative-HPLC on CSH® column using TFA method to give after lyophilization the expected compound.

Step 6: Example 24

To a solution of 2-[[(19S)-7-tert-butoxycarbonyloxy-10,19-diethyl-14,18-dioxo-17-oxa-3,13-diazapentacyclo[11.8,0.02,11.04,9.015,20]henicosa-1(21),2,4(9),5,7,10,1(20-heptaen-19-yl]oxycarbonyloxymethyl]-5-[[(2S)-2-[[(2S)-2-[6-(2,5-dioxopyrrol-1-yl)hexanoylamino]-3-methyl-butanoyl]amino]-5-ureido-pentanoyl]amino]benzenesulfonic acid (12.5 mg; 1.66 μmol) in DCM (2.5 mL), was added TFA (0.3 mL) and the solution was stirred at r.t. for 1.5 hours. The reaction was purified by C₁₈ reverse phase preparative-HPLC on CSH® column using TFA method to give after lyophilizaation Example 24. HRMS (ESI) [M+H]+ found=1071.3768 (δ=0.4 ppm)

### EXAMPLE 25

### (1S,3S)-3,5,12-trihydroxy-3-(hydroxyacetyl)-10-methoxy-6,11-dioxo-1,2,3,4,6,11-hexahydrotetracen-1-yl 2,3,6-trideoxy-3-[({[4-({N-[6-(2,5-dioxo-2,5-dihydro-1H-pyrrol-1-yl)hexanoyl]-L-valyl-N⁵-carbamoyl-L-ornithyl}amino)-2-sulfophenyl]methoxy}carbonyl)amino]-α-L-lyxo-hexopyranoside

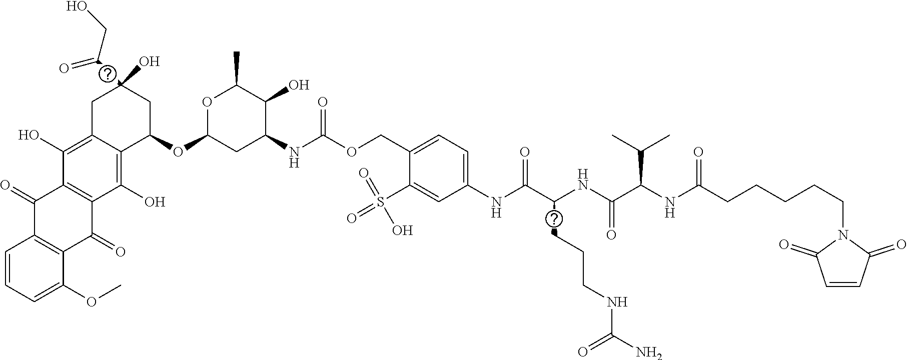 [SMILES: COc1cccc2c1C(=O)c1c(O)c3c(c(O)c1C2=O)C[C@@](O)(C(=O)CO)C[C@H]3O[C@@H]1C[C@H](NC(=O)OCc2ccc(NC(=O)[C@H](CCCNC(N)=O)NC(=O)[C@H](NC(=O)CCCCCN3C(=O)C=CC3=O)C(C)C)cc2S(=O)(=O)O)[C@H](O)[C@H](C)O1]

Step 1: 5-[[(2S)-2-[[(2S)-2-(9H-fluoren-9-ylmethoxycarbonylamino)-3-methyl-butanoyl]amino]-5-ureido-pentanoyl]amino]-2-[[(2S,3S,4S,6R)-3-hydroxy-2-methyl-6-[[(1S,3S)-3,5,12-trihydroxy-3-(2-hydroxyacetyl)-10-methoxy-6,11-dioxo-2,4-dihydro-1H-tetracen-1-yl]oxy]tetrahydropyran-4-yl]carbamoyloxymethyl]benzenesulfonic acid

To a solution of sodium 5-[[(2S)-2-[[(2S)-2-(9H-fluoren-9-ylmethoxycarbonylamino)-3-methyl-butanoyl]amino]-5-ureido-pentanoyl]amino]-2-[(4-nitrophenoxy) carbonyloxymethyl]benzenesulfonate (157 mg; 0.1854 mmol; obtained from Step 1 of Example 6) in DMF (11 mL), were added (7S,9S)-70[(2R,4S,5S,6S)-4-amino-5-hydroxy-6-methyl-tetrahydropyran-2-yl]oxy-6,9,11-trihydroxy-9-(2-hydroxyacetyl)-4-methoxy-8,10-dihydro-7H-tetracene-5,12-dione (107.5 mg; 0.1853 mmol) followed by DIEA (323 μL; 1.854 mmol). the red mixture turned to dark purple and, after 2 hours conversion, was complete. This solution was used as such in the next step.

Step 2: 5-[[(2S)-2-[[(2S)-2-amino-3-methyl-butanoyl]amino]-5-ureido-pentanoyl]amino]-2-[[(2S,3S,4S,6R)-3-hydroxy-2-methyl-6-[[(1S,3S)-3,5,12-trihydroxy-3-(2-hydroxyacetyl)-10-methoxy-6,11-dioxo-2,4-dihydro-1H-tetracen-1-yl]oxy]tetrahydropyran-4-yl]carbamoyloxymethyl]benzenesulfonic acid

To the solution of 5-[[(2S)-2-[[(2S)-2-(9H-fluoren-9-ylmethoxycarbonylamino)-3-methyl-butanoyl]amino]-5-ureido-pentanoyl]amino]-2-[[(2S,3S,4S,6R)-3-hydroxy-2-methyl-6-[[(1S,3S)-3,5,12-trihydroxy-3-(2-hydroxyacetyl)-10-methoxy-6,11-dioxo-2,4-dihydro-1H-tetracen-1-yl]oxy]tetrahydropyran-4-yl]carbamoyloxymethyl]benzenesulfonic acid as prepared in previous step, was added DEA (96 μL; 0.9268 mmol). The reaction was stirred at r.t. for 1 hour. Solvent was partially evaporated off and the crude was purified by C₁₈ reverse phase preparative-HPLC on X-Bridge® column using TFA method to give after lyophilization the expected compound IR (cm⁻¹): 3357, 2661, 1666/1608, 1585/1531, 1201, 1082, 1020, 763/709. RMN¹H (500 MHz, DMSO-d6) δ ppm 14.06 (s, 1H), 13.29 (s, 1H), 10.15 (s, 1H), 8.62 (d, 1H), 8.04 (m, 3H), 7.93 (d, 1H), 7.93 (t, 1H), 7.89 (3, 1H), 7.66 (dd, 1H), 7.61 (dd, 1H), 7.26 (d, 1H), 6.88 (d, 1H), 5.99 (t, 1H), 5.49 (brs, 1H), 5.37 (m, 2H), 5.25 (brs, 1H), 4.97 (t, 1H), (brm, 2H), 4.58 (s, 2H), 4.46 (q, 1H), 4.18 (q, 1H), 3.99 (s, 3H), 3.75 (m, 1H), 3.63 (m, 1H), 3.51 (bs, 1H), 2.99 (m, 2H), 2.99 (m, 2H), 2.16 (m, 2H), 2.07 (m, 1H), 1.87 (td, 1H), 1.72 (m, 1H), 1.61 (m, 1H), 1.5 (dt, 1H), 1.45 (m, 1H), 1.37 (m, 1H), 1.14 (d, 3H), 0.93 (2d, 6H)

Step 3: Example 25

To a solution of 5-[[(2S)-2-[[(2S)-2-amino-3-methyl-butanoyl]amino]-5-ureido-pentanoyl]amino]-2-[[(2S,3S,4S,6R)-3-hydroxy-2-methyl-6-[[(1S,3S)-3,5,12-trihydroxy-3-(2-hydroxyacetyl)-10-methoxy-6,11-dioxo-2,4-dihydro-1H-tetracen-1-yl]oxy]tetrahydropyran-4-yl]carbamoyloxymethyl]benzenesulfonic acid (5 mg; 4.7 μmol) in DMF (1 mL), were added DIEA (1.6 μL; 5.2 μmmol) and (2,5-dioxopyrrolidin-1-yl) 6-(2,5-dioxopyrrol-1-yl)hexanoate (1.6 mg; 5.2 μmol). The solution was stirred at r.t. for 12 hours. The reaction was purified by C₁₈ reverse phase preparative-HPLC on Xbridge® column using TFA method to give Example 25. HRMS (ESI) [M+H]+ found=1222.4153 (δ=1.7 ppm)

### EXAMPLE 26

sodium N-{[(9H-fluoren-9-yl)methoxy]carbonyl}-L-valyl-N⁵-carbamoyl-N-[4-(hydroxymethyl)-3-(2-sulfonatoethyl)phenyl]-L-orithinamide

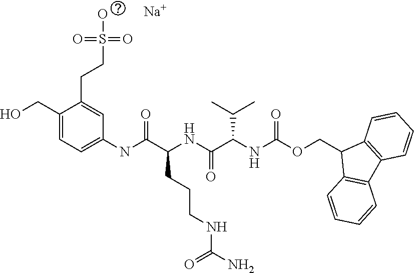 [SMILES: [Na+] ; CC(C)[C@H](NC(=O)OCC1c2ccccc2-c2ccccc21)C(=O)N[C@@H](CCCNC(N)=O)C(=O)Nc1ccc(CO)c(CCS(=O)(=O)O)c1]

Step 1: (2-allyl-4-nitro-phenyl)methoxy-tert-butyl-dimethyl-silane

A solution of tert-butyl-[(2-iodo-4-nitro-phenyl)methoxy]-dimethyl-silane (6.56 g; 16.68 mmol) and allyltributyltin (7.76 mL; 25.02 mmol) in 1,4-dioxane (165 mL) was purged with argon (3 times). Tetrakis(triphenylphosphine)palladium(0) (1.93 g; 1.67 mmol) was added and the mixture was purged again with argon (3 times) and stirred at 100° C. for 18 hours. The reaction mixture was filtered over Celtic® and concentrated to dryness. The residue was dissolved in DCM (150 mL) and washed with a 1M aqueous solution of sodium hydroxyde. The aqueous layer was extracted with DCM and the combined organic layers were washed with brine, dried over sodium sulfate, filtered and concentrated to dryness. The resulting crude was purified by flash chromatography over silica gel using cyclohexane/EtOAc (10/0 to 95/5) as eluent to afford the expected compound (4.91 g; 1597 mmol) as an orange oil. ¹H NMR (DMSO): δ0.11 (s, 6H), 0.92 (s, 9H), 3.49 (d, 2H, J=6.5 Hz), 4.84 (s, 2H), 5.04-5.15 (m, 2H), 5.92-6.02 (m, 1H), 7.69 (d, 1H, J=8.5 Hz), 8.02 (d, 1H, J=2.5 Hz), 8.13 (dd, 1H, J=2.5 and 8.5 Hz)

Step 2: 3-[2-[[tert-butyl(dimethyl)silyl]oxymethyl]-5-nitro-phenyl]propane-1,2-diol

To a solution of (2-allyl-4-nitro-phenyl)methoxy-tert-butyl dimethyl-silane (4.91 g; 15.97 mmol) in a mixture of acetone (147 mL) and water (20 mL), were added 4-methylmorpholine N-oxide (3.74 g; 31.94 mmol) and potassium osmate (VI) dihydrate (0.29 g; 0.8 mmol) and the mixture was stirred at r.t. for 20 hours. The reaction mixture was quenched with a saturated aqueous solution of sodium thiosulfate (40 mL) and water (40 mL) and the aqueous layer was extracted with EtOAc. The combined organic layers were washed with brine, dried over sodium sulfate, filtered, and concentrated to dryness. The resulting crude was purified by flash chromatography over silica gel using cyclohexane/EOAc (10/0 to 5/5) as eluent to afford the expected compound (4.65 g; 13.62 mmol) as a yellow oil. ¹H NMR (DMSO): δ0.11 (s, 6H), 0.93 (s, 9H), 2.58 (dd, 1H, J=8.8 and 14.2 Hz), 2.9 (dd, 1H, J=3.5 and 14.2 Hz), 3.26-3.30 (m, 1H), 3.36-3.41 (m, 1H), 3.61-3.68 (m, 1H), 4.7 (t, 1H, J=5.7 Hz), 4.75 (d, 1H, J=5.4 Hz), 4.84-4.93 (m, 2H), 7.65-7.67 (m, 1H), 8.08-8.11 (m, 2H)

Step 3: 2-[2-[[tert-butyl(dimethyl)silyl]oxymethyl]-5-nitro-phenyl]acetaldehyde

To a solution of 3-[2-[[tert-butyl(dimethyl)silyl]oxymethyl]-5-nitro-phenyl]propane-1,2-diol (3.05 g; 8.22 mmol) in a mixture of water (18 mL) and THF (51 mL) at 0° C., was added sodium periodate (5.27 g; 24.65 mmol) portion-wise. The reaction mixture was stirred for 2 hours from 0° C. to 10° C., then quenched with a saturated aqueous solution of sodium thiosulfate (50 mL) and water (50 mL). The aqueous layer was extracted with EtOAc. The combined organic layers were washed with brine, dried over sodium sulfate, filtered and concentrated to dryness at 30° C. to afford the expected compound (2.61 g; 8.22 mmol) as a yellow oil used as such for the next step ¹H NMR (DMSO): δ 0.08 (s, 6H), 0.90 (s, 9H), 4.04 (s, 2H), 4.72 (s, 2H), 7.7 (d, 1H, J=8.8 Hz), 8.13 (d, 1H, J=2.5 Hz), 8.18 (dd, 1H, J=2.4 and 8.5 Hz), 9.69 (s, 1H)

Step 4: 2-[2-[[tert-butyl(dimethyl)silyl]oxymethyl]-5-nitro-phenyl]ethanol

To a solution of 2-[2-[[tert-butyl(dimethyl)silyl]oxymethyl]-5-nitro-phenyl]acetaldehyde 2.61 g;) in MeOH (40 mL), was added at 0° C. sodium borohydride (0.47 g; 12.33 mmol). The reaction mixture was warmed to r.t. and stirred for 1 hour, then quenched by addition of brine (25 mL) and water (25 mL). The aqueous layer was extracted with EtOAc then the combined organic layers were washed with brine, dried over sodium sulfate, filtered, and concentrated to dryness. The resulting crude was purified by flash chromatography over silica gel using cyclohexane/EtOAc (10/0 to 6/4) to afford the expected compound (1.36 g; 4.36 mmol) as a yellow solid. ¹H NMR (DMSO): δ0.11 (s, 6H), 0.92 (s, 9H), 2.82 (t, 2H, J =6.4 Hz), 3.67 (q, 2H, J=5.9 Hz), 4.74 (t, 1H, J=5.1 Hz), 4.87 (d, 2H), 7.66 (d, 1H, J=8.6 Hz), 8.08-8.11 (m, 2H)

Step 5: [2-(2-bromoethyl)-4-nitro-phenyl]methoxy-tert-butyl-dimethyl-silane

To a solution of 2-[2-[[tert-butyl(dimethyl)silyl]oxymethyl]-5-nitro-phenyl]ethanol (1.26 g; 4.05 mmol) in THF (25 mL) at 0° C., were added TEA (1.12 mL; 8.09 mmol) and mesyl chloride (0.47 mL; 6.07 mmol). The reaction mixture was allowed to warm slowly to r.t., stirred for 5 hours, then filtered and rinsed with THF. The filtrate was added to a solution of lithium bromide (1.76 g; 20.23 mmol) in THF (25 mL) at 0° C. The reaction mixture was allowed to warm slowly to r.t., stirred for 4 days, then quenched with a saturated aqueous solution of ammonium chloride (50 mL). The aqueous layer was extracted with EtOAc. The combined organic layers were washed with brine, dried over sodium sulfate, filtered and concentrated to dryness to afford the expected compound (1.38 g; 4.03 mmol) as a yellow oil used as such for the next step. ¹H NMR (DMSO): δ 0.12 (s, 6H), 0.92 (s, 9H), 3.27 (t, 2H, J=7.2 Hz), 3.81 (t, 2H, J=7.2 Hz), 4.87 (s, 2H), 7.68 (d, 1H, J=8.4 Hz), 8.13-8.16 (m, 1H), 8.18-8.19 (m, 1H)

Step 6: [2-(2-bromethyl)-4-nitro-phenyl]methanol

To a solution of [2-(2-bromoethyl)-4-nitro-phenyl]methoxy-tert-butyl-dimethyl-silane (1.38 g) in THF (28 mL) and water (14 mL) at 0° C., was added acetic acid (42 mL; 737.28 mmol). The reaction mixture was allowed to warm slowly to r.t., stirred for 4 days, then diluted with water (100 mL). The aqueous layer was extracted with EtOAc. The combined organic layers were washed with brine, dried over sodium sulfate, filtered and concentrated to dryness. The resulting crude was purified by flash chromatography over silica gel using cyclohexane/EtOAc (10/0 to 6/4) as eluent to afford the expected compound (0.73 g; 2.81 mmol) as a beige solid. ¹H NMR (DMSO): δ 3.27 (t, 2H, J=7.2 Hz), 3.8 (t, 2H, J=7.2 Hz), 4.67 (s, 2H), 5.51 (brs, 1H), 7.7 (d, 1H, J=8.4 Hz), 8.11-8.14 (m, 1H), 8.15-8.16 (m, 1H)

Step 7: 2-[2-(hydroxymethyl)-5-nitro-phenyl]ethylsulfonyloxysodium

To a solution of [2-(2-bromoethyl)-4-nitro-phenyl]methanol (188 mg; 0.72 mmol) in EtOH (750 μL), were successively added a solution of sodium sulfite (273 mg; 2.17 mmol) in water (1.1 mL) and tetrabutylammonium iodide (13 mg; 0.036 mmol). The reaction mixture was heated at 70° C. under micro-waves irradiation for 35 minutes. The reaction mixture was diluted with water (20 mL) and washed with EtOAc. The aqueous layer was concentrated to dryness and the resulting crude was purified by flash chromatography over silica gel using DCM/MeOH (10/0 to 6/4) as eluent and then by flash chromatography over C₁₈ using ACN/water (2/98 to 34/66) as eluent to afford the expected compound (80 mg; 0.28 mmol) as a white solid. ¹H NMR (DMSO): δ 2.66-2.70 (m, 2H), 2.94-2.98 (m, 2H), 4.64 (d, 2H, J=5.3 Hz), 5.47 (t, 1H, J=5.3 Hz), 7.68 (d, 1H, J=8.5 Hz), 7.99 (d, 1H, J=2.3 Hz), 8.06 (dd, 1H, J=2.4 and 8.4 Hz)

Step 8: 2-[5-amino-2-(hydroxymethylphenyl]ethylsulfonyloxysodium

To a solution of [2-(2-bromoethyl)-4-nitro-phenyl]methanol (293 mg; 1.03 mmol) in MeOH (11 mL) and water (2 mL) was added Pd/C 10% (55 mg; 0.052 mmol) under argon. The reaction mixture was purged with hydrogen and stirred at r.t. under atmospheric pressure of hydrogen for 16 hours. The reaction mixture was filtered over a 40 μm PTFE filter, rinsed with a mixture of MeOH/water (1/1) and concentrated to dryness to afford the expected compound (274 mg; 1.03 mmol) as a yellow solid used as such for the next step. ¹H NMR (DMSO): δ 2.55-2.59 (m, 2H), 2.75-2.79 (m, 2H), 4.32 (d, 2H, J=5.3 Hz), 5.66 (t, 1H, J=5.2 Hz), 4.86 (brs, 2H), 6.33-6.35 (m, 2H), 6.93 (d, 1H, J=7.8 Hz)

Step 9: Example 26

To a solution of 2-[5-amino-2-(hydroxymethyl)phenyl]ethylsulfonyloxysodium (304 mg; 1.15 mmol) in DMF (6 mL), were added (2S)-2-[[(2S)-2-(9H-fluoren-9-ylmethoxycarbonylamino)-3-methyl-butanoyl]amino]-5-ureido-pentanoic acid (474 mg; 0.96 mmol), HATU (472 mg; 1.24 mmol) and sodium hydrogenocarbonate (160 mg; 1.91 mmol). The reaction mixture was stored as r.t. for 20 hours, diluted with water (50 mL) and washed with EtOAc. The aqueous layer was concentrated to dryness and purified by flash chromatography over C₁₈ using ACN/water+TFA (0.1%) (2/98 to 50/50) as eluent to afford after freeze-drying, Example 26 (83 mg; 0.11 mmol) as a pale yellow solid. ¹H NMR (DMSO): δ 0.84-0.88 (m, 6H), 1.32-1.49 (m, 2H), 1.54-1.74 (m, 2H), 1.94-2.04 (m, 1H), 2.58-2.62 (m, 2H), 2.83-2.87 (m, 2H), 2.90-3.05 (m, 2H), 3.93 (t, 1H,, J=8 Hz), 4.23-4.34 (m, 3H), 4.37-4.43 (m, 1H), 4.45 (s, 2H), 5.34 (brs, 2H), 5.98 (brs, 1H), 7.25 (d, 1H, J =8.1 Hz), 7.30-7.34 (m, 2H), 7.39-7.44 (m, 4H), 7.74 (t, 2H, J=7.2 Hz), 7.89 (d, 2H, J=7.5 Hz), 8.08 (d, 1H, J=7.5 Hz), 9.90 (s, 1H). LCMS (2-100 ACN/H₂O+0.1% TFA): 76.60% Tr=8.1 min. Negative mode 708.45 detected (M−H)

### EXAMPLE 27

### N-{3-[2-(2,5-dioxo-2,5-dihydro-1H-pyrrol-1-yl)ethoxy]propanoyl}-L-valyl-N-{4-[(5S,8S,11S,12R)-11-[(2S)-butan-2-yl]-12-(2-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-2-oxoethyl)-4,10-dimethyl-3,6,9-trioxo-5,8-di(propan-2-yl)-2,13-dioxa-4,7,10-triazatetradecan-1-yl]-3-(2-sulfoethylphenyl}-N⁵-carbamoyl-L-ornithinamide

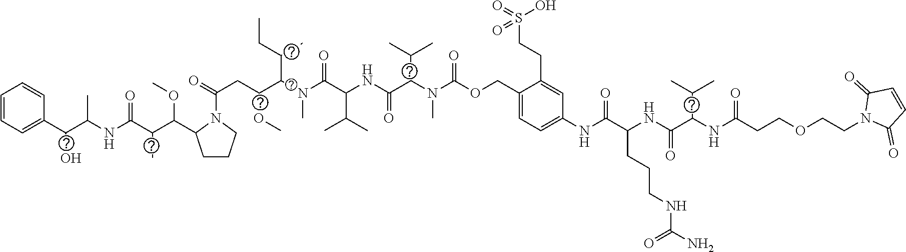 [SMILES: CCC(C)C(C(CC(=O)N1CCCC1C(OC)C(C)C(=O)NC(C)C(O)c1ccccc1)OC)N(C)C(=O)C(NC(=O)C(C(C)C)N(C)C(=O)OCc1ccc(NC(=O)C(CCCNC(N)=O)NC(=O)C(NC(=O)CCOCCN2C(=O)C=CC2=O)C(C)C)cc1CCS(=O)(=O)O)C(C)C]

Step 1: 2-[5-[[(2S)-2-[[(2S)-2-(9H-fluoren-9-ylmethoxycarbonylamino)-3-methyl-butanoyl]amino]-5-ureido-pentanoyl]amino]-2-[(4-nitrophenoxy)carbonyloxymethyl]phenyl]ethanesulfonic acid

To a solution of Example 26 (32 mg, 0.0436 mmol) in DMF (0.5 mL), were added bis(4-nitrophenyl) carbonate (53.1 mg; 0.1747 mmol) and DIEA (91 μL; 0.5240 mmol). The solution was stirred at r.t. for 4 hours. After evaporation to dryness under vacuum the crude was purified by chromatography on silica gel to lead to the expected compound.

Step 2: 2-[5-[[(2S)-2-[[(2S)-2-(9H-fluoren-9-ylmethoxycarbonylamino)-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenyl]ethanesulfonic acid

To a solution of MMAE (17 mg, 0.02368 mmol) in DMF (6 mL), were added 2-[5-[[(2S)-2-[[(2S)-2-(9H-fluoren-9-ylmethoxycarbonylamino)-3-methyl-butanoyl]amino]-5-ureido-pentanoyl]amino]-2-[(4-nitrophenoxy)carbonyloxymethyl]phenyl]ethanesulfonic acid (41.4 mg; 0.0473 mmol). DIEA (20 μL; 0.1184 mmol) and HOBt (6.4 mg; 0.047 mmol). The reaction was stirred at r.t. overnight and this solution was engaged as such in the next step.

Step 3: 2-[5-[[(2S)-2-[[(2)-2-amino-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenyl]ethanesulfonic acid, TFA

To the solution of 2-[5-[[(2S)-2-[[(2S)-2-(9H-fluoren-9-ylmethoxycarbonylamino)-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenyl]ethanesulfonic acid was added DEA (24 μL; 0.2338 mmol) and the solution was stirred at r.t. for 1.5 hours. The reaction was purified by C₁₈ reverse phase preparative-HPLC on CSH® column rising TFA method to give after lyophilization the expected compound.

Step 4: Example 27

To a solution of 2-[5-[[(2S)-2-[[(2S)-2-amino-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenyl]ethanesulfonic acid, TFA (9.5 mg; 7.1 mmol) in DMF (0.2 mL), was added DIEA (2.5 μL; 0.014 mmol) followed by (2,5-dioxopyrrolidin-1-yl) 3-[2-(2,5-dioxopyrrol-1-yl)ethoxy]propanoate (2.4 mg; 7.8 μmol). The reaction was stirred at r.t. overnight. The reaction was purified by C₁₈ reverse phase preparative-HPLC on CSH® column using TFA method to give after lyophilization Example 27. HRMS (ESI) [M+H]⁺ found=1426.7443 (δ=−6.7 ppm).

### EXAMPLE 28

### 5-[[(2S)-2-[[(2S)-2-[6-(3,4-dibromo-2,5-dioxo-pyrrol-1-yl)hexanoylamino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid

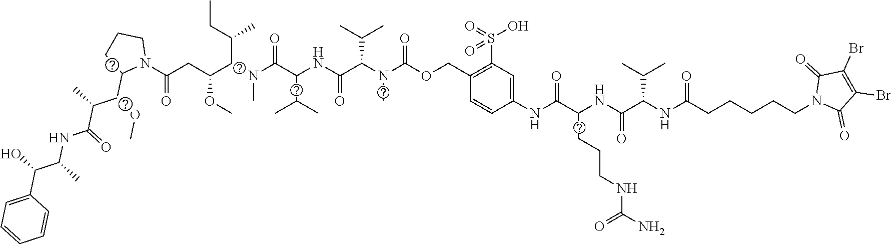 [SMILES: CC[C@H](C)C([C@@H](CC(=O)N1CCCC1C(OC)[C@@H](C)C(=O)N[C@H](C)[C@@H](O)c1ccccc1)OC)N(C)C(=O)C(NC(=O)[C@H](C(C)C)N(C)C(=O)OCc1ccc(NC(=O)C(CCCNC(N)=O)NC(=O)[C@@H](NC(=O)CCCCCN2C(=O)C(Br)=C(Br)C2=O)C(C)C)cc1S(=O)(=O)O)C(C)C]

To a solution of 6-(3,4-dibromo-2,5-dioxo-pyrrol-1-yl)hexanoic acid in solution in DMF (0.2 mL) were added HATU (11.3 mg; 29.8 μmol) as a powder. The reaction was stirred at r.t. for 10 minutes and lutidine (6.3 μL; 54.2 mmol) was added. The reaction was stirred at r.t. for 2 hours. then 5-[[(2S)-2-[[(2S)-2-amino-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid (10 mg; 8.3 μmol; obtained from Step 2 of Example 6) was added and the reaction was stirred at r.t. for 15 hours. the reaction was purified by C₁₈ reverse phase preparative-HPLC on X-Bridge® column using TFA method to give after lyophilization the expected compound. HRMS (ESI) [M+H]⁺ found=1552.5595 (δ=−2.5 ppm)

### EXAMPLE 34

### N-{[(9H-fluoren-9-yl)methoxy]carbonyl}-L-valyl-N⁵-carbamoyl-N-[4-(hydroxymethyl)-3-(3-sulfopropyl)phenyl]-L-ornithinamide

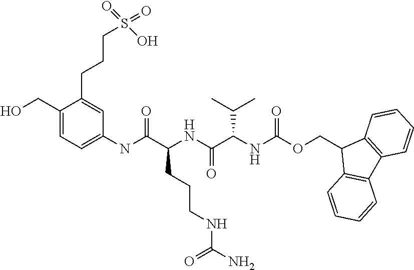 [SMILES: CC(C)[C@H](NC(=O)OCC1c2ccccc2-c2ccccc21)C(=O)N[C@@H](CCCNC(N)=O)C(=O)Nc1ccc(CO)c(CCCS(=O)(=O)O)c1]

Step 1: 2-iodo-4-nitrobenzoic acid

To a solution of 2-amino-4-nitrobenzoic acid (10.0 g; 54.9 mmol)) in ACN (280 mL), was added p-toluenesulfonic acid monohydrate (32.0 g; 168 mmol). The mixture was stirred at r.t for 15 minutes, then a solution of sodium nitrite (8.00 g; 115.9 mmol) and potassium iodide (24.0 g; 144.6 mmol) in water (140 mL) were added dropwise in 15 minutes. The reaction mixture was stirred tor 19 hours. After the completion of the reaction, the mixture was quenched with sodium thiosulfate (13.02 g; 82.36 mmol) and acidified with an aqueous solution of hydrogen chloride 3N (25 mL). The aqueous layer was extracted with DCM and the combined organic layers were washed with an aqueous solution of hydrogen chloride 1N, dried over sodium sulfate, filtered and concentrated to dryness to afford compound of the title (15.0 g; 51.2 mmol) as an orange powder. ¹H NMR (DMSO): 7.86 (d, 1H, J=8.4 Hz), 8.27 (d, 1H, J=8.4 Hz), 8.64 (s, 1H), 13.8 (brs, 1H)

Step 2: (2-iodo-4-nitrophenyl)methanol

To a solution of 2-iodo-4-nitrobenzoic acid (5.00 g; 17.1 mmol) in THF (70 mL), was added a solution of borane 1N in THF (85 mL; 85.0 mmol). The reaction mixture was stirred at 65° C. for 4 hours. After the completion of the reaction, the reaction mixture was cooled down to r.t. and quenched with MeOH (200 mL). The mixture was stirred at r.t. for 30 minutes then was concentrated to dryness. The resulting residue was purified by column chromatography on silica gel using cyclohexane/EtOAc (80/20 to 50/50) as eluent to afford compound of the title (3.38 g; 12.1 mmol) as a yellow solid. ¹H NMR (DMSO): δ 4.47 (d, 2H, J=5.2 Hz), 582 (t, 1H, J=5.2 Hz), 7.70 (d, 1H, J=8.8 Hz), 8.29 (dd, 1H, J=8.8 and 2.0 Hz), 8.54 (d, 1H, J=2.0 Hz).

Step 3: (4-amino-2-iodophenyl)methanol

To a solution of (2-iodo-4-nitrophenyl)methanol (3.70 g: 13.3 mmol) in EtOH (100 mL) and water (25 mL), were successively added iron (3.70 g; 66.3 mmol) and ammonium chloride (800 mg; 15.0 mmol). The reaction mixture was stirred for 3 hours at 80° C. After the completion of the reaction, the reaction mixture was filtered over Celite®, washed with EtOH and concentrated to dryness. The resulting residue was taken up in EtOAc and washed with a saturated solution of sodium hydrogen carbonate. The organic layer was dried over sodium sulfate, filtered and concentrated to dryness to afford compound of the title (2.48 g; 9.95 mmol) as a yellow oil. ¹H NMR (DMSO): δ 4.28 (d, 2H, J=5.2 Hz), 4.97 (t, 1H, J=5.2 Hz), 5.16 (s, 2H), 6.57 (d, 1H, J=8.4 Hz), 7.02-7.10 (m, 2H)

Step 4: 4-({[tert-butyl(dimethyl)silyl]oxy}methyl)-3-iodoaniline

To a solution of (4-amino-2-iodophenyl)methanol (3.51 g; 13.4 mmol) in DCM (150 mL), was added imidazole (0.95 g; 14.0 mmol). The mixture was cooled down to 0° C. then a solution of tert-butylchlorodimethylsilane (2.40 mL; 13.85 mmol) in DCM (150 mL) was added dropwise over 15 minutes. The ice bath was removed and the reaction mixture was stirred at r.t. for 16 hours. After the completion of the reaction, the reaction mixture was quenched with MeOH (20 mL) and concentrated to dryness. The resulting residue was purified by column chromatography on silica gel using cyclohexane/EtOAc (100/0 to 90/10) as eluent to afford compound of the title (3.64 g; 10.0 mmol) as a yellow oil. ¹H NMR (DMSO): δ 0.066 (s, 6H), 0.88 (s, 9H), 4.46 (s, 2H), 5.24 (s, 2H), 6.55 (d, 1H, J=8.4 Hz), 7.03 (d, 1H, J=8.4 Hz), 7.05 (s, 1H)

Step 5: N[4-({[tert-butyl(dimethyl)silyl]oxy}methyl)-3-iodophenyl]-N⁵-carbamoyl-N²-{[(9H-fluoren-9-yl)methoxy]carbonyl}-L-ornithinamide

To a solution of 4-({[tert-butyl(dimethyl)silyl]oxy}methyl)-3-iodoaniline (10.0 g; 27.5 mmol) in MeOH (70 mL) and DCM (140 mL) were successively added Fmoc-Cit-OH (12.0 g: 30.28 mmol) and EEDQ (8.17 g; 33.0 mmol). The reaction mixture was stirred for 14 hours at r.t. After the completion of the reaction, the resulting residue was purified by column chromatography on silica gel using DCM/MeOH (100/0 to 88/12) as eluent to afford compound of the title (17.09 g; 22.0 mmol) as a white solid. ¹H NMR (DMSO): δ 0.09 (s, 6H), 0.91 (s, 9H), 1.38-1.48 (m, 2H), 1.59-1.68 (m, 2H), 2.93-3.05 (m, 2H), 4.06-4.15 (m, 1H), 4.20-4.29 (m, 3H), 4.56 (s, 2H), 5.41 (s, 2H), 5.98 (t, 1H, J=5.5 Hz), 7.30-7.43 (m, 5H), 7.55 (dd, 1H, J=8.8 and 2.1 Hz), 7.69 (d, 1H, J=7.8 Hz), 7.74 (dd, 2H, J=7.2 and 3.4 Hz), 7.89 (d, 2H, J=7.5 Hz), 8.25 (d, 1H, J=1.5 Hz), 10.12 (s, 1H)

Step 6: N-[4-({[tert-butyl(dimethyl)silyl]oxy}methyl)-3-iodophenyl]-N⁵-carbamoyl-L-ornithinamide

To a solution of N-[4-({[tert-butyl(dimethyl)silyl]oxy}methyl)-3-iodophenyl]-N⁵-carbamoyl-N²-{[(9H-fluoren-9-yl)methoxy]carbonyl}-L-ornithinamide (17.1 g; 23.0 mmol) in THF (120 mL), was added dimethylamine 2M in THF (44.5 mL; 89.0 mmol). The reaction mixture was stirred for 15 hours at r.t. After concentration to dryness, the resulting residue was purified by column chromatography on C₁₈ using water/ACN (98/02 to 0/100) as eluent to afford compound of the title (5.47 g; 10.5 mmol) as a white solid. ¹H NMR (DMSO): δ 0.0 (s, 6H), 0.81 (s, 9H), 1.27-1.38 (m, 3H), 1.47-1.53 (m, 1H), 2.83-2.89 (m, 2H), 3.16-3.19 (m, 1H), 4.46 (s, 2H), 5.26 (s, 2H), 5.82 (t, 1H, J=5.6 Hz), 7.24 (d, 1H, J=8.5 Hz), 7.50 (dd, 1H, J=8.3 and 2.0 Hz), 8.17 (d, 1H, J=2.0 Hz)

Step 7: N-{[(9H-fluoren-9-yl)methoxy]carbonyl}-L-valyl-N-[4-({[tert-butyl(dimethyl)silyl]oxy}methyl)-3-iodophenyl]-N⁵-carbamoyl-L-ornithinamide

To a solution of N-[4-({[tert-butyl(dimethyl)silyl]oxy}methyl)-3-iodophenyl]-N⁵-carbamoyl-L-ornithinamide (3.00 g; 5.76 mmol) in 2-methyltetrahydrofuran (240 mL), were successively added Fmoc-Val-OSu (8.65 g; 8.65 mmol) and DIPEA (1.90 mL; 11.5 mmol). The reaction mixture was stirred for 15 hours at r.t. The reaction mixture was filtered through a sintered funnel and the recovered solid was washed with 2-methyltetrahydrofuran, then dried under high vacuum to afford compound of the title (3.57 g; 4.24 mmol) as a white solid. ¹H NMR (DMSO): δ 0.10 (s, 6H), 0.83-0.95 (m, 15H), 1.27-1.52 (m, 2H), 1.52-1.75 (m, 2H), 1.93-2.07 (m, 1H), 2.88-3.09 (m, 2H), 3.93 (t, 1H, J=8.0 Hz), 4.17-4.49 (m, 4H), 4.56 (s, 2H), 5.40 (s, 2H), 5.96 (t, 1H, J=5.6 Hz), 7.27-7.37 (m, 3H), 7.37-7.48 (m, 3H), 7.54 (d, 1H, J=8.0 Hz), 7.74 (t, 2H, J=7.2 Hz), 7.88 (d, 2H, J=7.6 Hz), 8.13 (d, 1H, J=7.6 Hz), 8.22 (s, 1H), 10.11 (s, 1H)

Step 8: N-{[(9H-fluoren-9-yl)methoxy]carbonyl}-L-valyl-N-[4-({[tert-butyl(diethyl)silyl]oxy}methyl)-3-(3-hydroxyprop-1-yn-1-yl)phenyl]-N⁵carbamoyl-L-ornithinamide

To a solution of N-{[(9H-fluoren-9-yl)methoxy]carbonyl}-L-valyl-N-[4-({[tert-butyl(dimethyl)silyl]oxy}methyl)-3-iodophenyl]-N⁵-carbamoyl-L-ornithinamide (4.0 g; 4.55 mmol) and 2-propyn-1-ol (0.54 mL; 9.10 mmol) in dry DMF (36 mL), was added DIPEA (2.26 mL; 13.66 mmol; 3.0 eq ). The reaction mixture was purged with Argon, then bis(triphenylphosphine)palladium(II) dichloride (0.64 g; 0.91 mmol) and copper(I) iodide (0.17 g; 0.91 mmol) were added. The reaction mixture was purged again with Argon, and stirred at r.t. for 16 hours. A saturated solution of ammonium chloride (80 mL) was added and the aqueous layer was extracted with EtOAc. The combined organic layers were washed with brine, dried over sodium sulfate, filtered and concentrated to dryness. The crude was purified by flash chromatography over silica gel using DCM/MeOH (10/0 to 85/15) as eluent to afford the product of the title (2.7 g; 3.52 mmol) as a brown solid. ¹H NMR (DMSO-d₆): 0.08 (s, 6H), 0.85-0.89 (m, 6H), 0.90 (s, 9H), 1.32-1.48 (m, 2H), 1.54-1.74 (m, 2H), 1.94-2.03 (m, 1H), 2.89-3.06 (m, 2H), 3.93 (dd, 1H, J=7.0 and 9.1 Hz), 4.22-4.33 (m, 5H), 4.36-4.41 (m, 1H), 4.74 (s, 2H), 5.35 (t, 1H, J=5.9 Hz), 5.40 (s, 2H), 5.96 (t, 1H, J=6.0 Hz), 7.32 (t, 2H, J=7.4 Hz), 7.36-7.43 (m, 4H), 7.48 (dd, 1H, J=2.3 and 8.8 Hz), 7.74 (t, 2H, J=7.7 Hz), 7.78 (s, 1H), 7.89 (d, 2H, J=7.8 Hz), 8.14 (d, 1H, J=7.1 Hz), 10.09 (s, 1H),

Step 9: N-{[(9H-fluoren-9-yl)methoxy]carbonyl}-L-valyl-N-[4-({[tert-butyl(dimethyl)silyl]oxy}methyl)-3-{3-[(methanesulfonyl)oxy]prop-1-yn-1-yl}phenyl]-N⁵-carbamoyl-L-ornithinamide

To a solution of N-{[(9H-fluoren-9-yl)methoxy]carbonyl}-L-valyl-N-[4-({[tert-butyl(dimethyl)silyl]oxy}methyl)-3-(3-hydroxyprop-1-yn-1-yl)phenyl]-N⁵-carbamoyl-L-ornithinamide (1.0 g; 1.3 mmol) in dry DMF (9 mL), were successively added at 0° C. DIPEA (0.64 mL; 3.9 mmol) and methanesulfonyl chloride (0.15 mL; 1.95 mmol). The reaction mixture was allowed to warm slowly to r.t. and stirred for 1 hour. A saturated solution of ammonium chloride (25 mL) was added, and the aqueous layer was extracted with EtOAc. The combined organic layers were washed with brine, dried over sodium sulfate, filtered and concentrated to dryness to afford the product of the title (1.1 g; 1.3 mmol) as an orange gum used as such for the next step. ¹H NMR (DMSO-d₆): 0.08 (s, 6H), 0.84-0.89 (m, 6H), 0.90 (s, 9H), 1.32-1.49 (m, 2H), 1.54-1.74 (m, 2H), 1.94-2.03 (m, 1H), 2.91-3.05 (m, 2H), 3.29 (s, 3H), 3.93 (dd, 1H, J=7.1 and 9.0 Hz), 4.20-4.33 (m, 3H), 4.37-4.42 (m, 1H), 4.75 (s, 2H), 5.23 (s, 2H), 5.4 (s, 2H), 5.96 (t, 1H, J=5.8 Hz), 7.32 (t, 2H, J=7.2 Hz), 7.39-7.43 (m, 4H), 7.56 (dd, 1H, J=2.3 and 8.8 Hz), 7.74 (t, 2H, J=7.4 Hz), 7.83 (d, 1H, J=2.3 Hz), 7.89 (d, 2H, J=7.3 Hz), 8.14 (d, 1H, J=7.3 Hz), 10.15 (s, 1H)

Step 10: N-{[(9H-fluoren-9-yl)methoxy]carbonyl}-L-valyl-N-{3-[3-(acetylsulfanyl)prop-1-yl-1-yl]-4-({[tert-butyl(dimethyl)silyl]oxy}methyl)phenyl}-N⁵-carbamoyl-L-ornithinamide

To a solution of N-{[(9H-fluoren-9-yl)methoxy]carbonyl}-L-valyl-N-[4-({[tert-butyl(dimethyl)silyl]oxy}methyl)-3-{3-[(methanesulfonyl)oxy]prop-1-yn-1-yl}phenyl]-N⁵-carbamoyl-L-ornithinamide (1.1 g; 1.3 mmol) in dry DMF (10 mL), was added potassium thioacetate (0.3 g; 2.6 mmol). The reaction mixture was stirred at r.t. for 16 hours. A saturated solution of sodium bicarbonate (35 mL) was added, and the aqueous layer was extracted with EtOAc. The combined organic layers were washed with brine, dried over sodium sulfate, filtered, and concentrated to dryness. The crude was purified by flash chromatography over silica gel using DCM/MeOH (10/0 to 9/1) as eluent to afford the product of the title (0.52 g; 0.62 mmol) as a yellowish solid. ¹H NMR (DMSO-d₆): 0.08 (s, 6H), 0.84-0.89 (m, 6H), 0.90 (s, 9H), 1.31-1.49 (m, 2H), 1.54-1.73 (m, 2H), 1.95-2.02 (m, 1H), 2.4 (s, 3H), 2.89-3.06 (m, 2H), 3.93 (dd, 1H, J=6.9 and 9.1 Hz), 3.97 (s, 2H), 4.20-4.33 (m, 3H), 4.36-4.41 (m, 1H), 4.69 (s, 2H), 5.39 (s, 2H), 5.96 (t, 1H, J=5.8 Hz), 7.32 (t, 2H, J=7.2 Hz), 7.35-7.43 (m, 4H), 7.49 (dd, 1H, J=2.2 and 8.5 Hz), 7.72-7.76 (m, 3H), 7.89 (d, 2H, J=7.5 Hz), 8.12 (d, 1H, J=7.4 Hz), 10.08 (s, 1H)

Step 11: N-{[(9H-fluoren-9-yl)methoxy]carbonyl}-L-valyl-N⁵-carbamoyl-N-[4-(hydroxymethyl)-3-(3-sulfoprop-1-yn-1-yl)phenyl]-L-ornithinamide

To a 35% solution of hydrogen peroxide (1.08 mL; 12.57 mmol), was added formic acid (9.68 mL; 256.5 mmol) at 0° C. This solution was stirred for 1 hour, then added to N-{[(9H-fluoren-9-yl)methoxy]carbonyl}-L-valyl-N-{3-[3-(acetylsulfanyl)prop-1-yn-1-yl]-4-({[tert-butyl(dimethyl)silyl]oxy}methyl)phenyl}-N⁵-carbamoyl-L-ornithinamide (367 mg; 0.44 mmol) at r.t. The reaction mixture was stirred for 2 hours, diluted with water (20 mL) and concentrated to dryness at 35° C. The residue was suspended in hydrochloric acid 1N (25 mL) and washed with DCM/MeOH (7/3). The aqueous layer was concentrated to dryness at 35° C. The crude was purified by flash chromatography over C₁₈ using ACN/water +TFA (0.1%) (2/98 to 50/50) as eluent to afford after freeze-drying product of the title (117 mg; 0.13 mmol) as a white solid. ¹H NMR (DMSO-d₆): δ0.84-0.88 (m, 6H), 1.33-1.50 (m, 2H), 1.55-1.75 (m, 2H), 1.95-2.02 (m, 1H), 2.89-3.05 (m, 2H), 3.58 (s, 2H), 3.91-3.95 (m, 1H), 4.20-4.33 (m, 3H), 4.37-4.42 (m, 1H), 4.55 (s, 2H), 5.22 (brs, 1H), 5.38 (s, 2H), 5.96 (t, 1H, J=5.6 Hz), 7.32 (t, 2H, J=7.2 Hz), 7.36-7.43 (m, 4H), 7.48 (dd, 1H, J=2.4 and 9.2 Hz), 7.65 (d, 1H, J=1.6 Hz), 7.74 (t, 2H, J=6.8 Hz), 7.89 (d, 2H, J=7.4 Hz), 8.12 (d, 1H, J=7.6 Hz), 10.03 (s, 1H). LCMS (2-100 ACN/H₂O+0.05% TFA): 77.27%, Rt =8.3 min. Positive mode 720.26 detected (M+H⁺)

Step 12: Example 34

To a suspension of N-{[(9H-fluoren-9-yl)methoxy]carbonyl}-L-valyl-N⁵-carbamoyl-N-[4-(hydroxymethyl)-3-(3-sulfoprop-1-yn-1-yl)phenyl]-L-ornithinamide (10 mg; 0.014 mmol) in THF (600 μL) and acetic acid (60 μL), was added under argon Pt/C dry 5% (7 mg; 0.0018 mmol). The reaction mixture was purged with hydrogen three times then stirred at r.t. for 64 hours. The reaction mixture was filtered over a 45 μm PTFE filter, washed with MeOH and MeOH/water (1/1), then concentrated to dryness to afford Example 34 (7 mg) as an orange oil. LCMS (2-100 ACN/H₂O+0.05% FA): 6.93%, Rt=8.8 min. Negative mode 722.19 detected (M−H)

As performed for Examples 6 to 12, Examples 14 to 25, Example 27 or Example 28. Example 34, a para-amino-benzyl linker compound of Formula (I), can be used for the preparation of Linker-Drug compound of Formula (II) and for the preparation of an antibody-drug conjugate of Formula (III) according to the invention.

### EXAMPLE A

### Conjugation and Analytical Characterization of ADCs

As used throughout this application, antibody drug conjugates can be identified using a naming convention in the general format of “target antigen/antibody-linker-drug”. For example only, if an antibody-drug conjugate is referred to as “Target X-Example Y”, such a conjugate would comprise an antibody that binds Target X, and a linker-drug exemplified in Example Y.
1. Conjugation

Exemplary ADCs were synthesized using one of the methods described below. Antibodies used in the exemplary ADC synthesis were defined by the abbreviations Ab T TG and Ab T (Table 1).

TABLE 1
Antibodies used for the purpose of the invention

| Antibody | | | Sequence | | |
|---|---|---|---|---|---|
| abbreviation | Antibody | Mutation | (VL/VH) | Patent | Allotype |
| Ab T TG | Trastuzumab | N297S | SEQ 1/2 | U.S. Pat. No. 6,870,034B2 | G1m17 |
| Ab T | Trastuzumab | WT | SEQ 1/2 | U.S. Pat. No. 6,870,034B2 | G1m17, 1 |

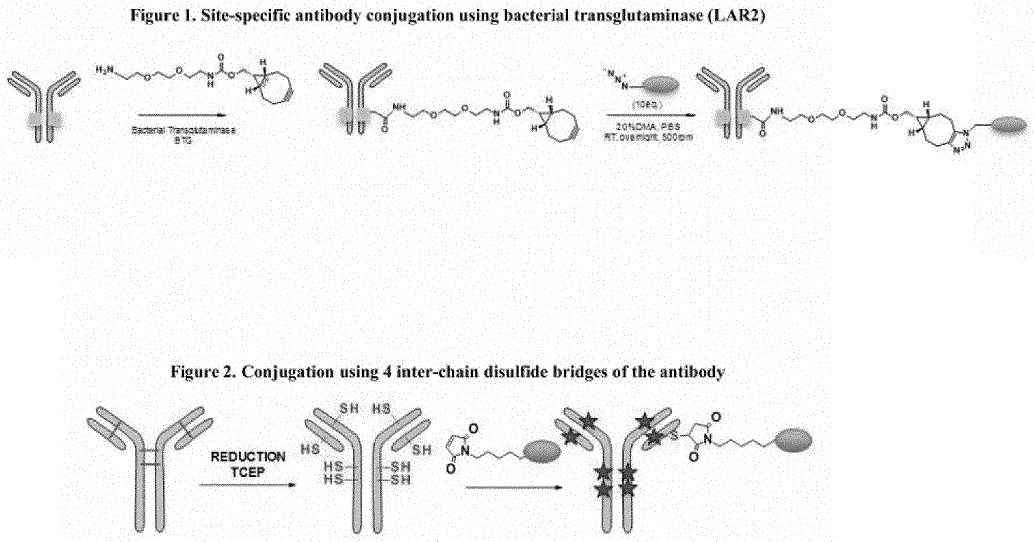

Antibody T TG was endowed with a bacterial transglutaminase (BTG)—reactive glutamines that were specifically functionalized with amine containing cyclooctyne BCN (or N-[(1R,8S,9s)-Bicyclo[6.1.0]non-4-yn-9-ylmethyloxycarbonyl]-1,8-diamino-3,6-dioxaoctane). The site specific antibody conjugation to BCN moiety using bacterial transglutaminase was performed as described in Innate Pharma 2013 (presentation at ADC Summit, San Francisco, Calif., Oct. 15, 2013), WO 2017/059160A1 and WO 2016/144608A1. These modifications allowed conjugating the described azide containing precursors using the following method A (FIG. 1).

The conjugates of Ab T were obtained by full reduction of the four inter-chain disulfide bonds followed by conjugation with an excess amount of drug (FIG. 2). For the purpose of the invention a water-soluble tertiary phosphine such as Tris-(2-carboxyethyl) phosphine (TCEP) was used as a reducing agent. Full reduction and conjugation produce primarily the fully loaded species with 8 drugs per antibody and eventually a small proportion of 6 drug loaded mAb explaining average DAR value of 7.1 and 7.7 (Table 2).

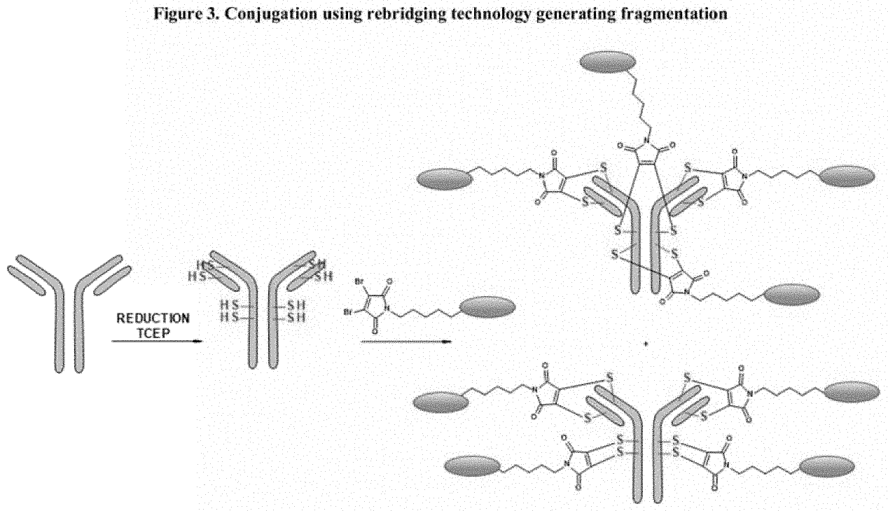

Some conjugates of Ab T were produced by rebridging technology described in Method D below. The four inter*chain disulfide bonds of the Ab T were fully reduced with TCEP and reformed using dibromo maleimide functionalized linker-payload (FIG. 3). The conjugation results in two conformations due to the proximity of the cysteine implicated in interchain disulfide bridges on each heavy chain of the antibody.

### Method A (DAR2)

Dimethyl sulfoxide (DMSO, 619 μl, 20% of the coupling volume) was added to the Ab solution (1.8 mg/mL; 2.5 mL, 4.5 mg). The mixture was stirred for 30 seconds in vortex followed by the addition of 4-fold molar excess of linker-warhead payload (20 mM, 6 μL in DMSO). The reaction was stirred at r.t. overnight at 64 rpm. To remove the unconjugated linker-payload, it was added 10-fold molar excess of DBCO-containing Tentagel resin (0.1-0.2 mmol/g, Iris Biotech, CS-0477.0500) and the mixture was stirred for 6 hours at r.t. The buffer was exchanged with PBS 1× (Sigma Life Science, P3813, 10PAK) by 3 filtration cycles using Vivaspin 20, 50KD, PES (Sartorius Stedim, VS2031), then filtered sterilely through 0.2 μm sterile PES Filter, 25 mm (Whatmann, G896-2502) and stored at 4° C.

### Method B (DAR8)

The Ab solution (10.3 mg/mL; 0.5 mL) was diluted in EDTA containing PBS buffer pH 7.4 (10 mM; 0.5 mL) to fix a final EDTA concentration in the coupling reaction of 5 mM. Then TCEP (1 mM in PBS buffer pH 7.4, 849 μL) was added to the antibody followed by incubation at 37° C. for 2 hours. After reduction, the antibody solution was cooled down to 2-8° C. and 20-fold molar excess of the linker-payload (5 mM, 141 μL in DMSO) was added. The reaction was incubated at 4° C. for 1.5 hours. The solution was centrifuged (14000G at 4° C.) for 20 minutes and it was loaded on HiLoad 26/600 Superdex 200 pg (GE Healthcare, 28989336) SEC chromatography column. The ADC was eluted with 20% DMA in PBS (Sigma Life Science, P3813, 10PAK) followed by 2 cycle's dialysis (16 and 4 hours) in PBS 1× pH 7.4 (Sigma Life Science, P3813, 10PAK). The conjugate was concentrated using Vivaspin 20, 50KDV PBS (Sartorius Stedim, VS2031), filtered sterilely through 0.2 μm sterile PES Filter, 25 mm (Whatmann, G896-2502) and stored at 4° C.

### Method C (DAR8)

The Ab solution (10.3 mg/mL; 0.5 mL) was diluted in EDTA containing PBS butter pH 7.4 (10 mM; 0.5 mL) to fix a final EDTA concentration in the coupling reaction of 5 mM. Then TCEP (1 mM in PBS buffer pH 7.4; 849 μL) was added to the antibody followed by incubation at 37° C. for 2 hours. After reduction, the antibody solution is cooled down to 2-8° C. and 20-fold molar excess of the linker-payload (5 mM; 141 μL in DMSO) was added. The reaction was incubated at 4° C. for 1.5 hours. Then the conjugate was purified on rmp protein A resin (GE Healthcare, 17-5138-01) followed by 2 cycle's dialysis (16 and 4 hours) in PBS 1× pH 7.4 (Sigma Life Science, P3813, 10PAK). The ADC was concentrated using Vivaspin 20, 50KD, PES (Sartorius Stedim, VS2031), filtered sterilely through 0.2 μm sterile PES Filter, 25 mm (Whatmann, G896-2502) and stored at 4° C.

### Method D (DAR4)

To Ab solution (5 mg/ml, 0.5 ml) in BBS buffer pH 8 (prepared as described below), was added 8-fold molar excess of TCEP (1 mM in BBS pH 8, 137 μl) and the reaction was incubated at 37° C. for 2 hours. Then, 7.5-fold molar excess of the linker-payload (1 mM, 129 μL in DMF) was added and the resulting solution was mixed at r.t. for 1 hour at 600 rpm. The resulting ADC was dialyzed in PBS 1× pH 7.4 (Sigma Life Science, P3813, 10PAK) for 16 hours at 4° C. Then the conjugate was purified on rmp protein A resin (GE Healthcare, 17-5138-01) followed by 2 hours dialysis at r.t. in PBS 1× pH 7.4 (Sigma Life Science, P3813, 10PAK). The ADC was concentrated using Vivaspin 20, 50KD, PES (Sartorius Stedim, VS2031), filtered sterilely through 0.2 μm sterile PES Filter, 25 mm (Whatmann, G896-2502) and stored at 4° C.

Preparation of BBS buffer pH 8: Na₂B₄O₇. 10 H₂O (528 mg) was dissolved in deionized water (27.7 mL). Sodium chloride (63 mg, 25 mM) and EDTA (16 mg, 1 mM) were solubilized in 22.3 mL of hydrochloric acid 0.1 M. Then, the two solutions were mixed together, and the resulting solution is directly used for the conjugation step.
2. LC-MS General Methodology

Drug-to-antibody ratio (DAR) of exemplary ADCs was determined by liquid chromatography hyphenated with mass spectrometry (LC-MS) with the following method LC-I or LC-II. For the LC-I method, mobile phase A was purified by MS grade water (Biosolve, Dieuze, France, 00232141B1BS), mobile phase B by MS grade acetonitrile (Biosolve, Dieuze, France, 0001204101BS) and mobile phase D purified by MS grade water supplemented with 1% of Formic acid (FA) (Honeywell/Fluka, Bucharest, Romania, 56302). Mobile phase D was fixed at 10% in order to maintain a 0.1% FA mobile phase composition and column temperature was set at 80° C. A general MS method was optimized for all ADCs synthesized (Table 2). For the LC-II method, mobile phase A was purified by MS grade water (Biosolve, Dieuze, France, 00232141B1BS) and mobile phase B by MS grade acetonitrile (Biosolve, Dieuze, France, 0001204101BS), both containing 0.1% formic acid (FA) (Honeywell/Fluka, Bucharest, Romania, 56302). The column temperature was set at 80° C. A general MS method was optimized for all ADCs synthesized (Table 2).

Method LC-1: ADC was loaded onto a Bioresolve RP mAb Polyphenyl, 450A, 2.7 μm, 2.1×150 mm (Waters, Saint-Quentin-en-Yvelines, France, 186008946). For analysis in both intact and reduced conditions, a desalting step was performed for 15 minutes at 20% of B with a flow rate of 0.6 mL min. Elution step was performed with a gradient from 1.5 min at 20% B to 16.5 minutes at 70% B with a flow rate of 0.3 mL/min. A wash step was set from 16.8 minutes to 18.8 minutes at 90% B with a flow rate of 0.6 mL/min. Finally, a conditioning step was used at 19.1 minutes for 1.9 minutes at 20% B with a flow rate of 0.6 mL/min (Total run time=21 minutes).

Method LC-II: ADC was loaded onto a Bioresolve RP mAb Polyphenyl, 450A, 2.7 μm, 2.1×150 mm (Waters, Saint-Quentin-en-Yvelines, France, 186008946). For analysis in both intact and reduced conditions, a desalting step was performed for 1.5 minutes at 20% of B with a flow rate of 0.6 ml/min. Then the elution step was carried out at the same flow rate with a gradient from 1.5 minutes at 20% B to 16.5 min at 50% B. The following washing step was set from 16.8 minutes to 18.8 minutes at 100% B with flow 0.6 mL/min. Finally, a conditioning step was used from 18.8 minutes to 192 minutes from 100% to 20% B stabilizing the column with additional 1.9 minutes at 20% B with a flow rate mentioned above (Total run time=21 min).

LC-MS analysis was performed using a Waters UPLC H-Class Bio chromatography system hyphenated with a Xevo G2 XS Q-TOF ESI mass spectrometer (Waters, Manchester, UK). The ADC was either analyzed in intact condition (no preliminary treatment) or following reduction with 5 mM (final concentration) of dithiothreitol DTT (Thermo Scientific, Rockford, Ill., 20291). Subsequently, treated ADC was analyzed using the aforementioned LC-I and LC-II (Table 2). Electrospray-ionization time-of-flight mass spectra of the analytes were acquired using MassLynx™ acquisition software (Waters, Manchester, UK). Then, the extracted intensity vs. m/z spectrum was deconvoluted using Maximum Entropy (MaxEnt) method of MassLynx™ software in order to deter mine the mass of each intact antibody species or each reduced antibody fragment depending on the treatment used. Finally, DAR was determined from the deconvoluted spectra or UV chromatogram by summing the integrated MS (total ion current) or UV (280 nm) peak area of unconjugated and conjugated given species (mAb or associated fragment). For the DAR determination by UV chromatogram, relative area percentage of every specie was multiplied by the number of drugs attached. The summed, weighted areas of every specie were divided by the sum of total relative area percentage and the results produced an estimation of the final average DAR value for the full ADC. For the DAR determination by deconvoluted spectra, the percentage of every specie identified was calculated by intensity peak value from deconvoluted spectra. The percentage obtained, was multiplied by the number of drugs attached. The summed results produced an estimation of the final average DAR value for the full ADC.
3. Size Exclusion Chomatography

Size exclusion chromatography (SEC) was performed to determine the quality of the ADCs, its aggregation percentage after purification. The analysis was performed on analytical column Superdex 200 Increase 5/150 GL (GE Healthcare, 28990945) in isocratic conditions 100% PBS pH 7.4 (Sigma Life Science, P3813, 10PAK), flow 0.45 mL/min for 12 minutes. The percentage of aggregate fraction in the conjugate sample was quantified based on the peak area absorbance at 280 nm. Its calculation was based on the ratio between the high molecular weight eluent at 280 nm divided by the sum of peak area absorbance at the same wavelength of the high molecular weight and monomeric eluents multiplied by 100.
4. Hydrophobic Interaction Chromatography

Hydrophobic Interaction Chromatography (HIC) was carried out to determine the hydrophobicity impact of the linker-payload and the bioconjugation technique on the antibody. The analysis was performed using TSKgel Butyl-NPR column (Tosoh Bioscience, 0014947) with mobile phase A (1.5 M ammonium sulfate (NH₄)₂S(₄, 25 mM potassium phosphate dibasic (K₂HPO₄), adjusted at pH 7) and B (25 mM potassium phosphate dibasic (K₂HPO₄), 20% isopropanol, adjusted at pH 7). The elution was started with flow of 0.6 mL/min at 5% B. The gradient raised from 5% to 100% B in 17 minutes, followed by washing step at 100% B for 5 minutes. Finally, a conditioning step was carried out 100%-5% B for 2 minutes followed by 2 minutes at 5% B.

For the purpose of the invention, a relative retention time (RRT) of each ADC was calculated using the ADC retention time (RT) divided by the antibody RT (Table 3).
5. Results

Characterization of the exemplary ADCs was summarized in Table 2 (named as Examples 15 to 22 and Examples 29 to 33; coupling and LC-MS method, aggregation status and DAR). The average DAR values were determined using the above LC-MS method LC-1 or LC-11 (point 2) and the percentage of aggregates was measured by size exclusion chromatography (SEC) described above (point 3).

TABLE 2
ADC Analytical characterization and coupling methodology

| | | Coupling | LC-MS | DAR | % Agg |
|---|---|---|---|---|---|
| Ex. | ADC | Method | Method | (by MS) | (by SEC) |
| 15 | Ab T TG-Example-8 | A | LC-I | 1.9 | 1.2 |
| 16 | Ab T-Example 6 | B | LC-I | 8 | 0.2 |
| 17 | Ab T-Example 7 | C | LC-I | 8 | 0.2 |
| 18 | Ab T-Example 10 | C | LC-I | 7.3 | 0.2 |
| 19 | Ab T-Example 9 | C | LC-I | 7.3 | 0.2 |
| 20 | Ab T-Example 11 | C | LC-I | 8 | 0.2 |
| 21 | Ab T-Example 12 | C | LC-II | 7.5 | 0.2 |
| 22 | Ab T-Example 14 | C | LC-II | 7.7 | 0.2 |
| 29 | Ab T-Example 23 | C | LC-II | 7.5 | 0.2 |
| 30 | Ab T-Example 27 | C | LC-II | 7.1 | 1.4 |
| 31 | Ab T-Example 24 | C | LC-II | 7.6 | 2.5 |
| 32 | Ab T-Example 25 | C | LC-II | 7.2 | 2.7 |
| 33 | Ab T-Example 28 | D | LC-II | 4 | 1.7 |

The hydrophobicity of the exemplified ADC was compared by the calculation of the relative retention time using HIC chromatography described above (point 4). Table 3 summarized RRT values of the conjugates.

TABLE 3
Comparison of ADC hydrophobicity using
the RRT by HIC chromatography

| | | | RT ADC | RT mAb |
|---|---|---|---|---|
| Ex. | ADC | RRT | (HIC) | (HIC) |
| 15 | Ab T TG-Example 8 | 0.97 | 5.84 | 6.01 |
| 16 | Ab T-Example 6 | 2.02 | 11.21 | 5.54 |
| 17 | Ab T-Example 7 | 2.31 | 12.06 | 5.22 |
| 18 | Ab T-Example 10 | 2.18 | 11.47 | 5.25 |
| 19 | Ab T-Example 9 | 2.15 | 11.31 | 5.25 |
| 20 | Ab T-Example 11 | 2.20 | 11.74 | 5.33 |
| 21 | Ab T-Example 12 | 2.65 | 13.01 | 4.91 |
| 22 | Ab T-Example 14 | 2.33 | 10.03 | 4.75 |
| 29 | Ab T-Example 23 | 2.83 | 14.02 | 4.96 |
| 30 | Ab T-Example 27 | 2.56 | 14.16 | 5.54 |
| 31 | Ab T-Example 24 | 1.45 | 8.05 | 5.54 |
| 32 | Ab T-Example 25 | 1.28 | 6.83 | 5.32 |
| 33 | Ab T-Example 28 | 1.85 | 8.58 | 4.63 |

Stability study was performed for all exemplified ADCs, where the conjugates were incubated in conditions of accelerated degradation in PBS buffer at +37° C. over 1 week. SEC chromatography was exploited to measure the aggregation status of the conjugates. The obtained results were illustrated in Table 4.

TABLE 4
Stability studies of the ADCs at conditions
of accelerated degradation in PBS buffer

| | | % Agg (by SEC) | % Agg (by SEC) |
|---|---|---|---|
| Ex. | ADC | T₀ | 1 week 37° C. |
| 15 | Ab T TG-Example 8 | 1.2 | 1.2 |
| 16 | Ab T-Example 6 | 0.2 | 0.2 |
| 17 | Ab T-Example 7 | 0.2 | 0.2 |
| 18 | Ab T-Example 10 | 0.2 | 5.2 |
| 19 | Ab T-Example 9 | 0.2 | 4.3 |
| 20 | Ab T-Example 11 | 0.2 | 1.8 |
| 21 | Ab T-Example 12 | 0.2 | 2.7 |
| 22 | Ab T-Example 14 | 0.2 | 1.7 |
| 29 | Ab T-Example 23 | 0.2 | 1.1 |
| 30 | Ab T-Example 27 | 1.4 | 8.3 |
| 31 | Ab T-Example 24 | 2.5 | 11.6 |
| 32 | Ab T-Example 25 | 2.7 | 21.6 |
| 33 | Ab T-Example 28 | 1.7 | 1.9 |

### EXAMPLE B

### Effect of Exemplified ADCs on Cell Viability using CTG Assay

HCC1954 (HER+) and MOLT4 (HER−) cell lines were cultivated in RPM1 supplemented with 10% heat inactivated fetal bovine serum, penicillin (100 IU/ml), streptomycin (100 μg/ml) and L-glutamine (2 mM). Cell lines were cultured at 37° C. in a humidified atmosphere containing 5% CO₂. Cells were seeded in 96 microwell plates and exposed to the ADCs for 120 hours (5 fold serially diluted: 9 concentrations each, triplicates). Effects of ADCs on cell viability were assessed after 5 days of incubation at 37° C./5% CO₂ by quantification of cellular ATP levels using CellTiterGlo at 75 μL reagent/well. All the conditions were tested in triplicates. Luminescence was quantified on a multipurpose plate reader. IC₅₀s were calculated using standard four-parametric curve fitting. IC₅₀ is defined as the compound concentration at which the CTG signal is reduced to 50% of that measured tor the control. Each experiment was performed at least twice, with results being reproducible.

As shown in Table 5, all exemplified ADC are active in antigen-positive cells in CTG assay.

TABLE 5
Effect of exemplified ADCs on cell viability using CTG assay

| | HCC1954 (HER2+) | | | MOLT4 (HER2−) | | |
|---|---|---|---|---|---|---|
| Ex. | IC₅₀ n1 (M) | IC₅₀ n2 (M) | Mean (M) | IC₅₀ n1 (M) | IC₅₀ n2 (M) | Mean (M) |
| 15 | 2.38E−10 | 3.46E−10 | 2.92E−10 | >3.00E−07 | >3.00E−07 | >3.00E−07 |
| 16 | 1.22E−11 | 5.94E−11 | 3.58E−11 | 5.40E−08 | 6.27E−08 | 5.84E−08 |
| 17 | 2.53E−11 | 3.86E−11 | 3.20E−11 | 2.25E−07 | 1.47E−07 | 1.86E−07 |
| 18 | 4.24E−11 | 9.28E−11 | 6.76E−11 | 1.41E−07 | 2.83E−07 | 2.12E−07 |
| 19 | 4.55E−11 | 7.52E−11 | 6.04E−11 | 4.74E−08 | 2.21E−07 | 1.34E−07 |
| 20 | 4.53E−11 | 6.93E−11 | 5.73E−11 | 3.34E−08 | 6.21E−08 | 4.78E−08 |
| 21 | 7.73E−11 | 9.14E−11 | 8.44E−11 | 4.38E−08 | 9.78E−08 | 7.08E−08 |
| 22 | 2.25E−11 | 3.64E−11 | 2.94E−11 | 1.30E−08 | 1.20E−08 | 1.25E−08 |
| 29 | 4.80E−11 | 5.70E−11 | 5.25E−11 | 1.03E−07 | 9.02E−08 | 9.65E−08 |
| 30 | 1.37E−11 | 1.39E−11 | 1.38E−11 | 5.81E−08 | 4.78E−08 | 5.30E−08 |

1-25. (canceled)

26. A para-amino-benzyl linker compound of Formula (I): 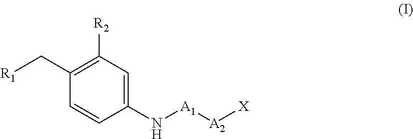
wherein:
R₁ represents a hydroxy group or a halogen atom;
R₂ represents a —S(O)₂(OH) group, a —S(O)₂(O⁻M⁺) group, a linear or branched —(C₁-C₄)alkyl-S(O)₂(OH) group, a linear or branched —(C₁-C₄)alkyl-S(O)₂(O⁻M⁺) group, a linear or branched -halo (C₁-C₄)alkyl-S(O)₂(OH) group, or a linear or branched -halo(C₁-C₄)alkyl-S(O)₂(O⁻M⁺) group;
A₁ represents a —C(O)—CH(R₃)—NH— group;
A₂ represents a —C(O)—CH(R₄)—NH— group, a 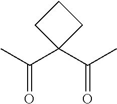
group, a 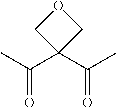
group, or a 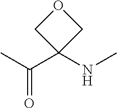
group;
R₃ and R₄, independently of one another, represent a side chain of an amino acid;
X represents a hydrogen atom, a hydroxy group, or a protecting group; and
M⁺ represents a pharmaceutically acceptable monovalent cation.

27. The para-amino-benzyl linker compound according to claim 26, wherein R₁ represents a hydroxy group, a bromine atom, a chlorine atom, or an iodine atom.

28. The para-amino-benzyl linker compound according to claim 26, wherein R₂ represents a —S(O)₂(OH) group, a —S(O)₂(O⁻M⁺) group, a —CH₂—S(O)₂(OH) group, a —CH₂—S(O)₂(O⁻M⁺) group, a —CH₂—CH₂—S(O)₂(OH) group, a —CH₂—CH₂—S(O)₂(O⁻M⁺) group, a —CH₂—CH₂—CH₂—S(O)₂(OH) group, or a —CH₂—CH₂—CH₂—S(O)₂(O⁻M⁺) group.

29. The para-amino-benzyl linker compound according to claim 26, wherein A₁ represents a —C(O)—CH(R₃)—NH— group, wherein R₁ represents a —(CH₂)₃—NH—CO—NH₂ group or a methyl group.

30. The para-amino-benzyl linker compound according to claim 26, wherein A₂ represents a —C(O)—CH(R₄)13 NH— group, wherein R₄ represents an isopropyl group. 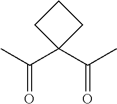
group; a 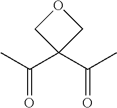
group; or a 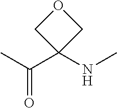
group.

31. The para-amino-benzyl linker compound according to claim 26, wherein A₁ represents a —C(O)—CH(R₃)—NH— group and A₂ represents a —C(O)—CH(R₄)—NH— group, wherein R₃ and R₄, independently of one another, represent a side chain of an amino acid.

32. The para-amino-benzyl linker compound according to claim 26, which is selected from the group consisting of:
sodium 5-[[(2S)-2-[[(2S)-2-(9H-fluoren-9-ylmethoxycarbonylamino)-3-methyl-butanoyl]amino]-5-ureido-pentanoyl]amino]-2-(hydroxymethyl)benzenesulfonate;
sodium 5-[[(2S)-2-[[(2S)-2-(9H-fluoren-9-ylmethoxycarbonylamino)-3-methyl-butanoyl]amino]propanoyl]amino]-2-(hydroxymethyl)benzenesulfonate;
5-[[(2S)-2-[[(2S)-2-(9H-fluoren-9-ylmethoxycarbonylamino)-3-methyl-butanoyl]amino]propanoyl]amino]-2-(hydroxymethyl)benzenesulfonic acid;
sodium [5-[[(2S)-2-[[(2S)-2-(9H-fluoren-9-ylmethoxycarbonylamino)-3-methyl-butanoyl]amino]-5-ureido-pentanoyl]amino]-2-(hydroxymethyl)phenyl]methanesulfonate;
2-(chloromethyl)-5-[[(2S)-2-[[(2S)-2-(9H-fluoren-9-ylmethoxycarbonylamino)-3-methyl-butanoyl]amino]-5-ureido-pentanoyl]amino]benzenesulfonic acid;
2-(chloromethyl)-5-[[(2S)-2-[[(2S)-2-(9H-fluoren-9-ylmethoxycarbonylamino)-3-methyl-butanoyl]amino]propanoyl]amino]benzenesulfonic acid;
5-[[(2S)-2-[[(2S)-2-(9H-fluoren-9-ylmethoxycarbonylamino)-3-methyl-butanoyl]amino]-5-ureido-pentanoyl]amino]-2-(iodomethyl)benzenesulfonic acid;
sodium N-{[(9H-fluoren-9-yl)methoxy]carbonyl}-L-valyl-N⁵-carbamoyl-N-[4-(hydroxymethyl)-3-(2-sulfonatoethyl)phenyl]-L-ornithinamide;
N-{[(9H-fluoren-9-yl)methoxy]carbonyl}-L-valyl-N⁵-carbamoyl-N-[4-(hydroxymethyl)-3-(3-sulfopropyl)phenyl]-L-ornithinamide.

33. A method for preparing an antibody-drug conjugate employing the para-amino-benzyl linker compound of Formula (I) according to claim 26.

34. A Linker-Drug compound of Formula (II): 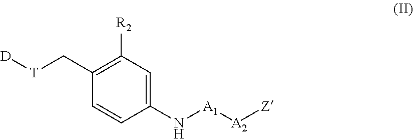
wherein:
D represents a drug moiety;
T is a bond, —O—C(O)—N(CH₃)—CH₂—CH₂—N(CH₃)—C(O)—*, —O—*, —NR₅—*, —NR₅—C(O)—*, or —O—C(O)—*, wherein the * indicates the point of attachment to D;
R₂ represents a —S(O)₂(OH) group, a —S(O)₂(O⁻M⁺) group, a linear or branched —(C₁-C₄)alkyl-S(O)₂(OH) group, a linear or branched —(C₁-C₄)alkyl-S(O)₂(O⁻M⁺) group, a linear or branched -halo(C₁-C₄)alkyl-S(O)₂(OH) group, or a linear or branched -halo(C₁-C₄)alkyl-S(O)₂(O⁻M⁺) group;
A₁ represents a —C(O)—CH(R₃)—NH— group;
A₂ represents a —C(O)—CH(R₄)—NH— group, a 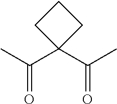
group, a 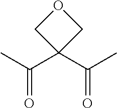
group, or a 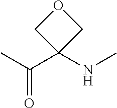
group;
R₃ and R₄, independently of one another, represent a side chain of an amino acid;
R₅ represents a hydrogen atom or a (C₁-C₄)alkyl group;
Z′ represents a Spacer unit precursor; and
M⁺ represents a pharmaceutically acceptable monovalent cation.

35. The Linker-Drug compound according to claim 34, wherein R₂ represents a —S(O)₂(OH) group, a —CH₂—S(O)₂(OH) group, a —CH₂—S(O)₂O⁻M⁺) group, a —CH₂—CH₂—S(O)₂(OH) group, a —CH₂—CH₂—S(O)₂(O⁻M⁺) group, a —CH₂—CH₂—CH₂—S(O)₂(OH) group, or a —CH₂—CH₂—CH₂—S(O)₂(O⁻M⁺) group.

36. The Linker-Drug compound according to claim 34, wherein A₁ represents a —C(O)—CH(R₃)—NH— group, wherein R₃ represents a —CH₂)₃—NH—CO—NH₂ group or a methyl group.

37. The Linker-Drug compound according to claim 34, wherein A₂ represents a —C(O)—CH(R₄)—NH— group, wherein R₄ represents an isopropyl group; 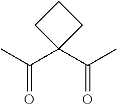
group; a 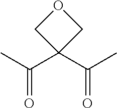
group; or a 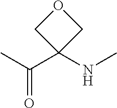
group.

38. The Linker-Drug compound according to claim 34, wherein T represents a bond or —O—C(O)—*, wherein the * indicates the point of attachment to D.

39. The Linker-Drug compound according to claim 34, wherein Z′ represents: 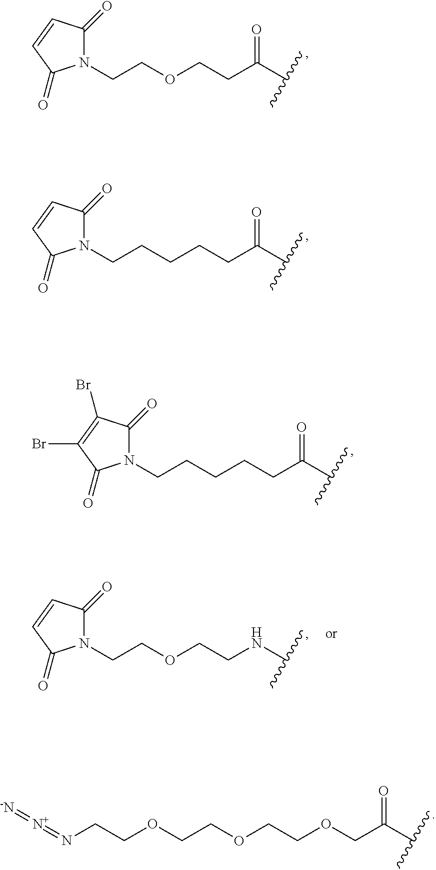
wherein the wavy line indicates the covalent attachment site to the N-terminus or the carbonyl group of the A₂ group.

40. The Linker-Drug compound according to claim 34, which is selected from the group consisting of: 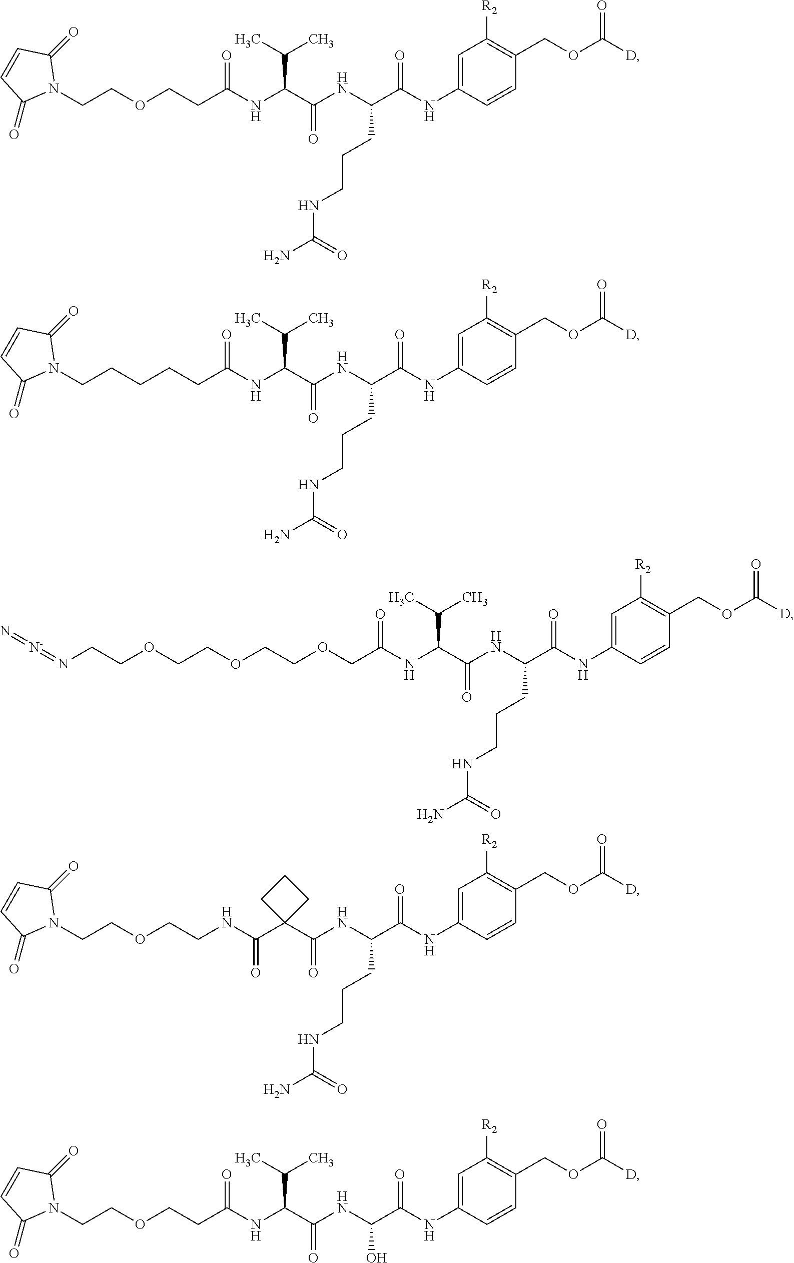 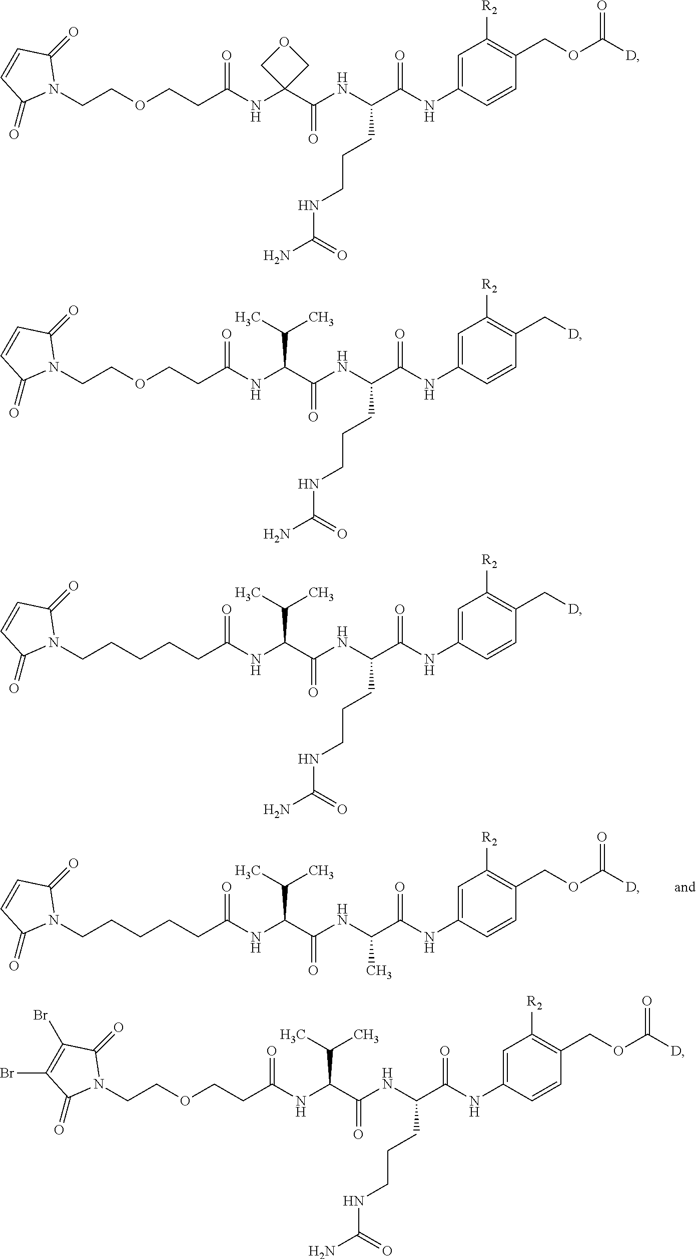
wherein R₂ and D are as defined in claim 34.

41. The Linker-Drug compound according to claim 34, which is selected from the group consisting of:
sodium 5-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate;
sodium 5-[[(2S)-2-[[(2S)-2-[6-(2,5-dioxopyrrol-1-yl)hexanoylamino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate;
sodium 5-[[(2S)-2-[[(2S)-2-[[2-[2-[2-(2azidoethoxy)ethoxy]ethoxy]acetyl]amino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate;
5-[[(2S)-2-[[1-[2-[2-(2,5-dioxopyrrol-1-yl)ethoxy]ethylcarbamoyl]cyclobutanecarbonyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid;
sodium 5-[[(2S)-2-[[(2S)-2-[3-[2-(2,5-dioxopyrrol-1-yl)ethoxy]propanoylamino]-3-methyl-butanoyl]amino]propanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonate;
5-[[(2S)-2-[[(2S)-2-[3-[2-(2,5-dioxopyrrol-1-yl)ethoxy]propanoylamino]-3-methyl-butanoyl]amino]propanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid;
5-[[(2S)-2-[[3-[2-[2-(2,5-dioxopyrrol-1-yl)ethoxy-]ethylcarbamoyl]oxetane-3-carbonyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid;
[5-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenyl]methanesulfonic acid;
[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]-2-sulfo-phenyl]methyl-[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-dimethyl-ammonium; 2,2,2-trifluoroacetate;
N-({[4-({N-[6-(2,5-dioxo-2,5-dihydro-1H-pyrrol-1-yl)hexanoyl]-L-valyl-L-alanyl}amino)-2-sulfophenyl]methoxy}carbonyl)-N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide;
N-[6-(2,5-dioxo-2,5-dihydro-1H-pyrrol-1-yl)hexanoyl]-L-valyl-N⁵-carbamoyl-N-(4-{[({[(4S)-4,11-diethyl-9-hydroxy-3,14-dioxo-3,4,12,14-tetrahydro-1H-pyrano[3′,4′:6,7]indolizino[1,2-b]quinolin-4-yl]oxy}carbonyl)oxy]methyl}-3-sulfophenyl)-L-ornithinamide;
(1S,3S)-3,5,12-trihydroxy-3-(hydroxyacetyl)-10-methoxy-6,11-dioxo-1,2,3,4,6,11-hexahydrotetracen-1-yl 2,3,6-trideoxy-3-[({[4-({N-[6-(2,5-dioxo-2,5-dihydro-1H-pyrrol-1-yl)hexanoyl]-L-valyl-N⁵-carbamoyl-L-ornithyl}amino)-2-sulfophenyl]methoxy}carbonyl)amino]-α-L-lyxo-hexopyranoside;
N-{3-[2-(2,5-dioxo-2,5-dihydro-1H-pyrrol-1-yl)ethoxy]propanoyl}-L-valyl-N-{4-[(5S,8S,11S,12R)-11-[(2S)-butan-2-yl]-12-(2-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-2-oxoethyl)-4,10-dimethyl-3,6,9-trioxo-5,8-di(propan-2-yl)-2,13-dioxa-4,7,10-triazatetradecan-1-yl]-3-(2-sulfoethyl)phenyl}-N⁵carbamoyl-L-ornithinamide; and
5-[[(2S)-2-[[(2S)-2-[6-(3,4-dibromo-2,5-dioxo-pyrrol-1-yl)hexanoylamino]-3-methyl-butanoyl]amino]-5-ureido-pentanoyl]amino]-2-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidon-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]benzenesulfonic acid.

42. An antibody-drug conjugate of Formula (III): 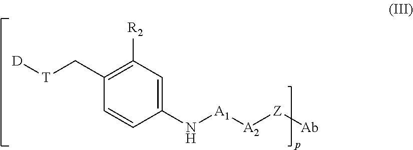
wherein
Ab represents an antibody or an antigen-binding fragment thereof;
D is a drug moiety;
T is a bond, —O—C(O)—N(CH₃)—CH₂—CH₂—N(CH₃)—C(O)—*, —O—*, —NR₅—*, —NR₅—C(O)—*, or —O—C(O)—*, wherein the * indicates the point of attachment to D;
Z represents a Spacer Unit;
A₁ represents a —C(O)—CH(R₃)—NH— group:
A₂ represents a —C(O)—CH(R₄)—NH— group, a 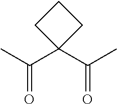
group, a 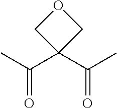
group, or a 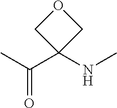
group;
R₂ represents a —S(O)₂(OH) group, a —S(O)₂(O⁻M⁺) group, a linear or branched —(C₁-C₄)alkyl-S(O)₂(OH) group, a linear or branched —(C₁-C₄)alkyl-S(O)₂(O⁻M⁺) group, a linear or branched -halo(C₁-C₄)alkyl-S(O)₂(OH) group, or a linear or branched -halo(C₁-C₄)alkyl-S(O)₂(O⁻M⁺) group;
R₃ and R₄, independently of one another, represent a side chain of an amino acid;
R₅ represents a (C₁-C₄)alkyl group;
M⁺ represents a pharmaceutically acceptable monovalent cation, and
p is an integer from 1 to 8.

43. The antibody-drug conjugate according to claim 42, wherein T is a bond or —O—C(O)—*, wherein the * indicates the point of attachment to D.

44. The antibody-drug conjugate according to claim 42, wherein R₂ represents a —S(O)₂(OH) group, a —S(O)₂(O⁻M⁺) group, a —CH₂—S(O)₂(OH) group, a —CH₂—S(O)₂(O⁻M⁺) group, a —CH₂—CH₂—S(O)₂(OH) group, a —CH₂—CH₂—S(O)₂(O⁻M⁺) group, a —CH₂—CH₂—CH₂—S(O)₂(OH) group, or a —CH₂—CH₂—CH₂—S(O)₂(O⁻M⁺) group.

45. The antibody-drug conjugate according to claim 42, wherein A₁ represents a —C(O)—CH(R₃)—NH— group in which R₃ represents a —(CH₂)₃—NH—CO—NH₂ group or a methyl group.

46. The antibody-drug conjugate according to claim 42, wherein A₂ represents a —C(O)—CH(R₄)—NH— group in which R₄ represents an isopropyl group; a 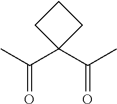
group; a 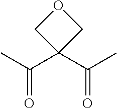
group; or a 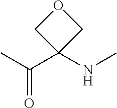
group.

47. The antibody-drug conjugate according to claim 42, wherein the antibody-drug conjugate is formed from a Linker-Drug compound of Formula (II) selected from: 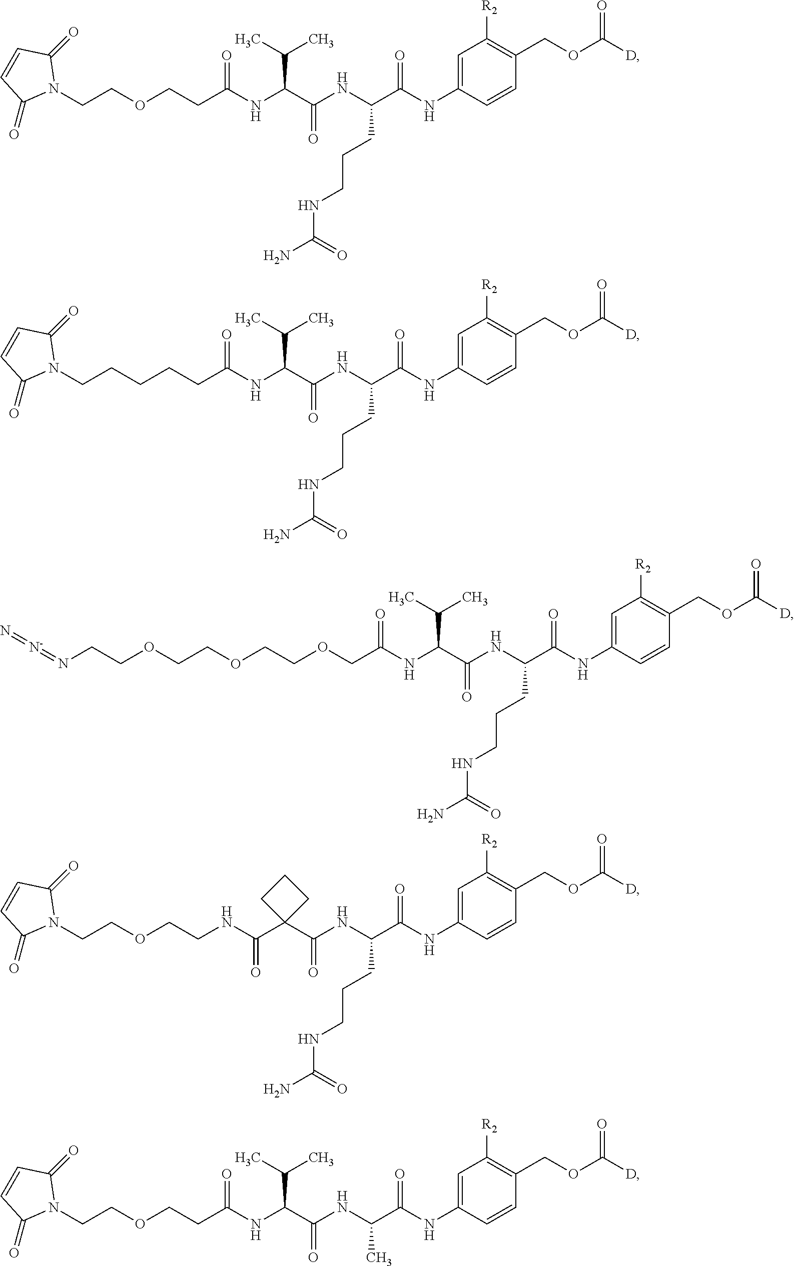 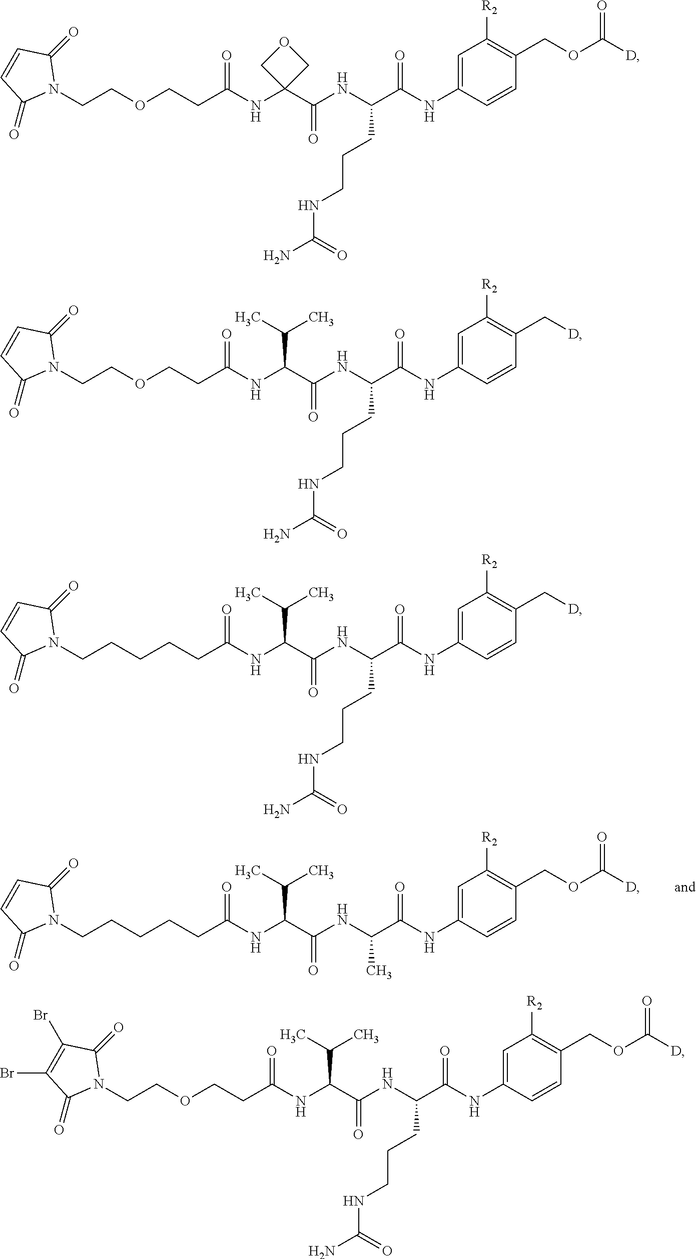
wherein R₂ and D are as defined in claim 42.

48. The antibody-drug conjugate according to claim 42, wherein the antibody-drug conjugate comprises a Formula selected from: 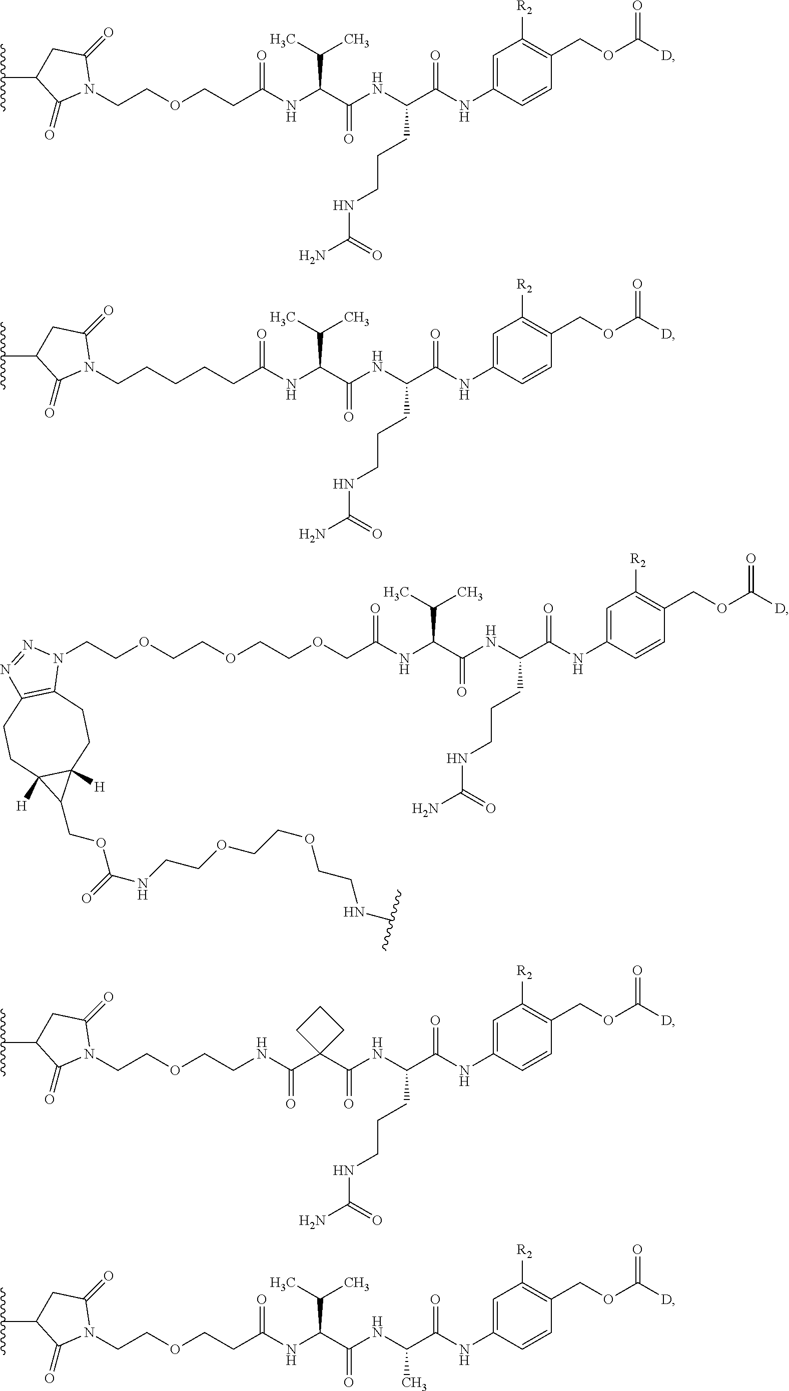 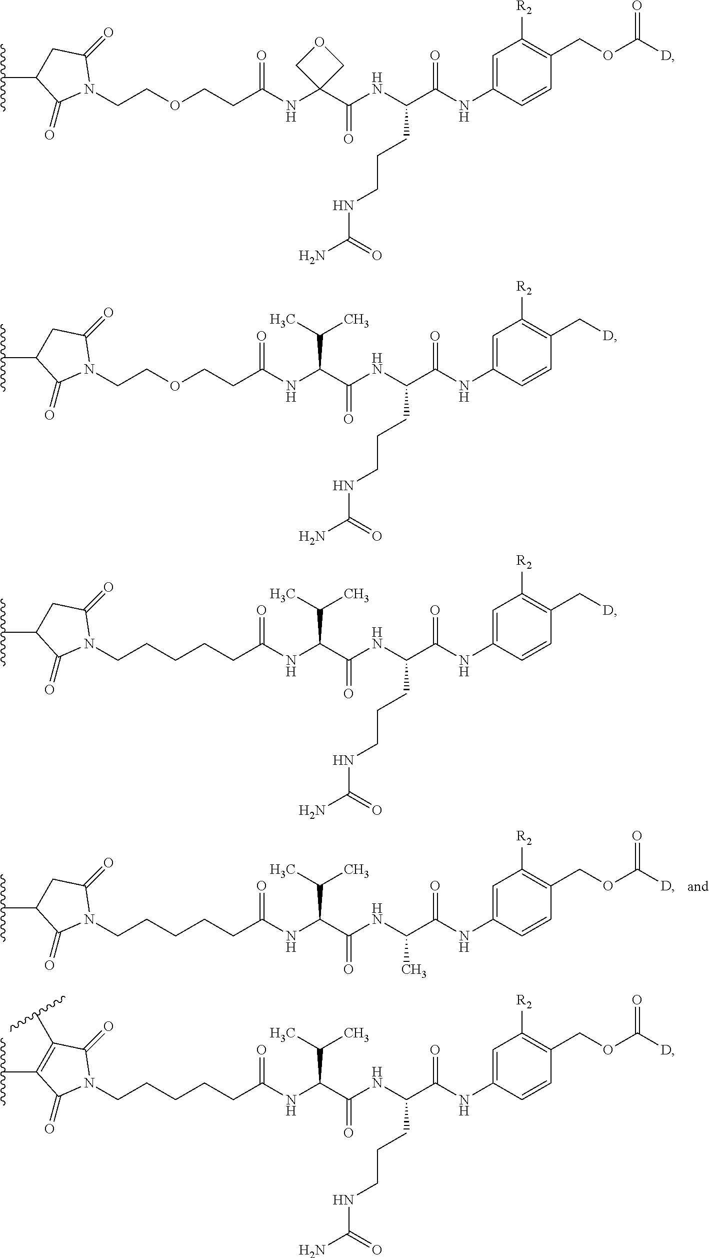
wherein the wavy line indicates the covalent attachment site to the antibody or antigen-binding fragment thereof and, D and R₂ are as defined in claim 42.

49. A pharmaceutical composition comprising the antibody-drug conjugate according to claim 42, and a pharmaceutically acceptable carrier.

50. A method of treating a condition requiring an antibody-drug conjugate in a mammal in need thereof, comprising administration of the antibody-drug conjugate according to claim 42, alone or in combination with one or more pharmaceutically acceptable carriers.
